(19) United States
(12) Patent Application Publication
(10) Pub. No.: US 2005/0171095 A1
(43) Pub. Date: Aug. 4, 2005

(54) Combination of CRF antagonists and 5-HT1B receptor antagonists

(75) Inventors: Harry R. Howard JR., Bristol, CT (US); Yuhpyng L. Chen, Waterford, CT (US)

Correspondence Address: PFIZER INC, 150 EAST 42ND STREET, 5TH FLOOR - STOP 49, NEW YORK, NY, 10017-5612, US

(73) Assignee: Pfizer Inc

(21) Appl. No.: 11/026,782

(22) Filed: Dec. 31, 2004

Related U.S. Application Data
(60) Provisional application No. 60/534,511, filed on Jan. 6, 2004.

(51) Int. Cl. A61K 31/541; A61K 31/519; A61K 31/513

(52) U.S. Cl. 514/227.5; 514/252.12; 514/265.1; 514/269

(57) ABSTRACT

The present invention relates to a pharmaceutical composition for treating, for example, a disorder or condition selected from the group consisting of hypertension, depression, generalized anxiety disorder, phobias, posttraumatic stress disorder, avoidant personality disorder, sexual dysfunction, eating disorders, obesity, chemical dependencies, cluster headache, migraine, pain, Alzheimer's disease, obsessive-compulsive disorder, panic disorder, memory disorders, Parkinson's diseases, endocrine disorders, cerebellar ataxia, gastrointestinal tract disorders, negative symptoms of schizophrenia, premenstrual syndrome, Fibromyalgia Syndrome, stress incontinence, Tourette syndrome, trichotillomania, kleptomania, male impotence, cancer, chronic paroxysmal hemicrania and headache in a mammal, preferably a human, comprising (i) a corticotropin releasing factor antagonist or a pharmaceutically acceptable salt thereof,
(ii) a 5-HT_{1B} receptor antagonist or a pharmaceutically acceptable salt thereof, wherein the 5-HT_{1B} receptor antagonist is selected from the group consisting of (A) a compound of the formula I as described in the specification and (B) a compound of the formula II as described in the specification, and optionally (iii) a pharmaceutically acceptable carrier.

### BACKGROUND OF THE INVENTION

The present invention relates to pharmaceutical compositions containing corticotropin releasing factor antagonists or pharmaceutically acceptable salts thereof and 5-HT_{1B} receptor antagonists or pharmaceutically acceptable salts thereof, and to their medicinal use for treating disorders associated with the central nervous system.

U.S. Pat. Nos. 6,464,028, 6,258,953, 6,380,186, 6,323,229, 6,197,773, 6,451,803, 6,403,592, 6,472,388, 6,562,813 and 6,627,627 and U.S. Patent Publication Nos. 2002/0091119 and 2003/0083337 describe certain aralkyl and aralkylidene heterocyclic lactams and imides that are 5-HT_{1B} receptor antagonists and that are used in the compositions of the present invention. Other 5-HT₁ receptor antagonists are described in European Patent Publications 701,819, 434,561 and 343,050, PCT publications WO 94/21619, WO 95/31988, and WO 96/00720, Glennon et al., “5-HT_{1D} Serotonin Receptors”, Clinical Drug Res. Dev., 22, 25-36 (1991), and G Maura et al., J. Neurochem, 66 (1), 203-209 (1996). These references describe 5-HT₁ receptor antagonists, including 5-HT_{1B} receptor antagonists, as useful in the treatment of, for example, migraine, depression, obsessive compulsive disorder, post-traumatic stress disorder (PTSD), and eating disorders, as well as other disorders associated with the central nervous system.

Corticotropin releasing factor (CRF) antagonists are another class of therapeutic agents that have been described as effective in the treatment of certain disorders or conditions. CRF antagonists are disclosed in U.S. Pat. Nos. 4,605,642 and 5,063,245. Other CRF antagonists are disclosed in International patent publications WO 95/33750; WO 95/34563; WO 94/13661; WO 94/13644; WO 94/13643; WO 94/13676; WO 94/13677; WO 95/33727; WO 98/05661; WO 98/08847; WO 98/08846; and European patent publications EP 778277 and EP 773023. Yet other CRF antagonists are disclosed in the following patent publications: EP 576350; EP 659747; EP 812831; WO 95/10506; WO 96/35689; WO 96/39400; WO 97/00868; WO 97/14684; WO 97/29109; WO 97/29110; WO 97/35539; WO 97/35580; WO 97/35846; WO 97/44038; WO 97/45421; WO 98/03510; WO 98/08821; WO 98/11075; WO 98/15543; WO 98/21200; WO 98/27066; WO 98/29397; WO 98/29413; WO 98/42699; WO 98/35967; WO 98/42706; WO 98/45295; WO 98/47874; WO 98/47903; WO 98/51312; WO 99/01454; WO 99/01439; WO 99/10350; WO 99/12908; WO 99/00373; WO 99/38868; WO 99/51597; WO 99/51599; WO 99/40089; WO 99/51598; and WO 99/51600. Still more CRF antagonists are disclosed in U.S. Pat. Nos. 5,109,111; 5,132,111; 5,245,009; 5,464,847; 5,493,006; 5,510,458; 5,644,057; 5,663,292; 5,668,145; 5,705,646; 5,712,303; and 5,723,608. An overview of the patent literature on CRF antagonists is provided in T. E. Christos and A. Arvanitis, Exp. Opin. Ther. Patents (1998) 8(2):143-152. Many of the above cited publications include information on how to make the CRF antagonists described therein. The importance of CRF antagonists is also set out in, e.g., P. Black, Scientific American: “Science & Medicine,” 1995, 2:16-25; T. Lovenberg, et al., Current Pharmaceutical Design, 1995, 1: 305-316; D. T. Chalmers et al., Trends in Pharmacological Sciences, April 1996, pages 166-172; and U.S. Pat. No. 5,063,245. An outline of the activities possessed by CRF antagonists is found in M. J. Owens et al., 1991, Pharm. Rev., 43:425-473.

In particular, CRF antagonists have been described as effective in the treatment of, for example, stress-related illnesses; mood disorders such as depression, including, for example, depression in cancer patients, depression in Parkinson's patients, Postmyocardial Infarction depression, depression in patients with human immunodeficiency virus (HIV), Subsyndromal Symptomatic depression, depression in infertile women, pediatric depression, major depression, single episode depression, recurrent depression, child abuse induced depression, post partum depression, DSM-IV major depression, treatment-refractory major depression, severe depression, psychotic depression, post-stroke depression, neuropathic pain, manic depressive illness, including manic depressive illness with mixed episodes and manic depressive illness with depressive episodes, seasonal affective disorder, bipolar depression BP I, bipolar depression BP II, or major depression with dysthymia; chronic fatigue syndrome; dysthymia; pain perception, such as fibromyalgia; gastrointestinal diseases; hemorrhagic stress; ulcers; stress-induced psychotic episodes; fever; diarrhoea; post-operative ileus; colonic hypersensitivity; irritable bowel syndrome; Chron's disease; spastic colon; inflammatory disorders such as rheumatoid arthritis and osteoarthritis; pain; asthma; psoriasis; allergies; osteoporosis; premature birth; hypertension; congestive heart failure; sleep disorders; neurogenerative diseases such as Alzheimer's disease, senile dementia of the Alzheimer's type, multiinfarct dementia, and Huntington's disease; head trauma; ischemic neuronal damage; excitotoxic neuronal damage; epilepsy; stroke; spinal cord trauma; psychosocial dwarfism; euthyroid sick syndrome; syndrome of inappropriate antidiuretic hormone; obesity; infertility; cancer; muscular spams; urinary incontinence; hypoglycemia and immune dysfunctions, including stress-induced immune dysfunctions, immune suppressions, and human immunodeficiency virus infections; stress-induced infections; anxiety disorders, including, for example, generalized anxiety disorder, panic disorder, post-traumatic stress disorder (PTSD), and social anxiety disorder; phobias, including, for example, agoraphobia, social phobia or simple phobias; eating disorders, including, for example, anorexia nervosa or bulimia nervosa; chemical dependencies and addictions, including, for example, addictions to alcohol, cocaine, amphetamine and other psychostimulants, morphine, heroin and other opioid agonists, phenobarbital and other barbiturates, nicotine, and diazepam and other benzodiazepines; drug and alcohol withdrawal symptoms; Parkinson's diseases, including, for example, dementia in Parkinson's disease, neuroleptic-induced parkinsonism or tardive dyskinesias; migraine; obsessive compulsive disorder; and headache, including, for example, headache associated with vascular disorders. See, for example, P. Black, Scientific American, 1995, 2:16-25; T. Lovenberg, et al., Current Pharmaceutical Design, 1995, 1:305-316; D. T. Chalmers et al., Trends in Pharmacological Sciences, April 1996, pages 166-172; M. J. Owens et al., Pharm. Rev., 1991, 43:425-473; and U.S. Pat. No. 5,063,245.

### SUMMARY OF THE INVENTION

The present invention relates to a pharmaceutical composition for treating, for example, a disorder or condition selected from the group consisting of hypertension, depression, generalized anxiety disorder, phobias, posttraumatic stress disorder, avoidant personality disorder, sexual dysfunction, eating disorders, obesity, chemical dependencies, cluster headache, migraine, pain, Alzheimer's disease, obsessive-compulsive disorder, panic disorder, memory disorders, Parkinson's diseases, endocrine disorders, cerebellar ataxia, gastrointestinal tract disorders, negative symptoms of schizophrenia, premenstrual syndrome, Fibromyalgia Syndrome, stress incontinence, Tourette syndrome, trichotillomania, kleptomania, male impotence, cancer, chronic paroxysmal hemicrania and headache in a mammal, preferably a human, comprising:
(i) a corticotropin releasing factor antagonist or a pharmaceutically acceptable salt thereof,
(ii) a 5-HT_{1B} receptor antagonist or a pharmaceutically acceptable salt thereof, wherein the 5-HT_{1B} receptor antagonist is selected from the group consisting of
(A) a compound of the formula I: 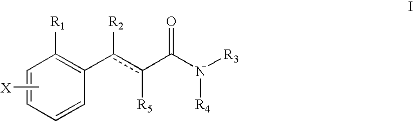
   wherein, in formula I:
R¹ is a group of the formula G¹, G², G³, G⁴, G⁵, G⁶ or G⁷ depicted below, 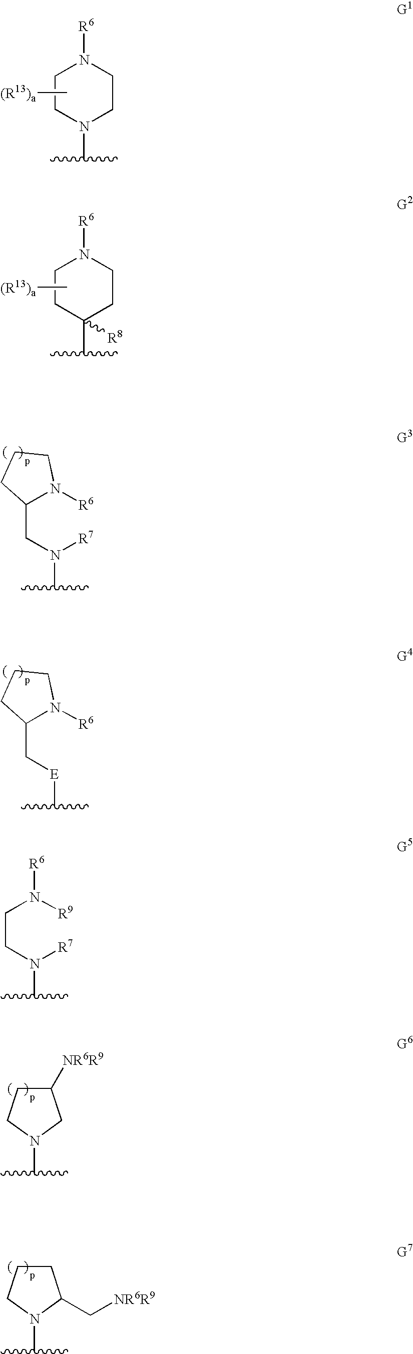
   a is zero to eight;
each R¹³ is, independently, (C₁-C₄)alkyl or a (C₁-C₄)methylene bridge from one of the ring carbons of the piperazine or piperidine ring of G¹ or G², respectively, to the same or another ring carbon or a ring nitrogen of the piperazine or piperidine ring of G¹ or G², respectively, having an available bonding site, or to a ring carbon of R⁶ having an available bonding site;
E is oxygen, sulfur, SO or SO₂;
X is hydrogen, chloro, fluoro, bromo, iodo, cyano, (C₁-C₆)alkyl, hydroxy, trifluoromethyl, (C₁-C₆)alkoxy, —SOₜ(C₁-C₆)alkyl wherein t is zero, one or two, —CO₂R¹⁰ or —CONR¹¹R¹²,
R² is hydrogen, (C₁-C₄)alkyl, phenyl or naphthyl, wherein said phenyl or naphthyl is optionally substituted with one or more substituents independently selected from the group consisting of chloro, fluoro, bromo, iodo, (C₁-C₆)alkyl, (C₁-C₆)alkoxy, trifluoromethyl, cyano and —SOₖ(C₁-C₆)alkyl wherein k is zero, one or two;
R³ is —(CH₂)ₘB, wherein m is zero, one, two or three and B is hydrogen, phenyl, naphthyl or a 5 or 6 membered heteroaryl group containing from one to four hetero-atoms in the ring, and wherein each of the foregoing phenyl, naphthyl and heteroaryl groups is optionally substituted with one or more substituents independently selected from the group consisting of chloro, fluoro, bromo, iodo, (C₁-C₆)alkyl, (C₁-C₆)alkoxy, (C₁-C₆)alkoxy-(C₁-C₆)alkyl-, trifluoromethyl, trifluoromethoxy, cyano, hydroxy, —COOH and —SOₙ(C₁-C₆)alkyl wherein n is zero, one or two;
R⁴ is (C₁-C₆)alkyl or C₆-C₁₀ aryl;
or R³ and R⁴ may optionally be taken together with the nitrogen to which they are attached to form a five to seven membered heteroalkyl ring, wherein any two of the carbon atoms of said heteroalkyl ring is optionally replaced with a heteroatom selected from the group consisting of nitrogen, oxygen or sulfur (e.g., pyrrolidine, isoxazolidine, 1,3-oxazolidin-3-yl, isothiazolidine, 1,3-thiazolidin-3-yl, 1,2-pyrazolidin-2-yl, 1,3-pyrazolidin-1-yl, piperidine, thiomorpholine, 1,2-tetrahydrothiazin-2-yl, 1,3-tetrahydrothiazin-3-yl, tetrahydrothiadiazine, morpholine, 1,2-tetrahydrodiazin-2-yl, 1,3-tetrahydrodiazin-1-yl, piperazine, etc.); wherein said heteroalkyl ring may optionally be substituted by aryl or heteroaryl (e.g., furyl, thienyl, thiazolyl, pyrazolyl, isothiazolyl, oxazolyl, isoxazolyl, pyrrolyl, triazolyl, tetrazolyl, imidazolyl, 1,3,5-oxadiazolyl, 1,2,4-oxadiazolyl, 1,2,3-oxadiazolyl, 1,3,5-thiadiazolyl, 1,2,3-thiadiazolyl, 1,2,4-thiadiazolyl, pyridyl, pyrimidyl, pyrazinyl, pyridazinyl, 1,2,4-triazinyl, 1,2,3-triazinyl, 1,3,5-triazinyl, benzoxazolyl, benzothiazolyl, benzisothiazolyl, benzisoxazolyl, benzimidazolyl, thianaphthenyl, isothianaphthenyl, benzofuranyl, isobenzofuranyl, isoindolyl, indolyl, indazolyl, isoquinolyl, quinolyl, phthalazinyl, quinoxalinyl, quinazolinyl, benzoxazinyl; etc.);
R⁵ is hydrogen, (C₁-C₆)alkyl or aryl, wherein aryl is selected from the group consisting of phenyl, naphthyl, pyridyl or pyrimidyl, wherein any of said aryl is optionally independently substituted on any available bonding site by any of the radicals of X;
or R⁵ and R⁴ taken together form a divalent group —Yₙ₂—;
Y is selected from the group consisting of (a) CR⁴R⁵, wherein R⁴ and R⁵ are independently selected from hydrogen, (C₁-C₆)alkyl and trifluoromethyl; (b) a phenylene, naphthylene or a 5 or 6 membered heteroarylene ring comprising containing from one to four hetero-atoms in the heteroarylene ring, and wherein each of the foregoing phenylene, naphthylene and heteroarylene rings may optionally be substituted with one or more substituents independently selected from the group consisting of chloro, fluoro, bromo, iodo, (C₁-C₆)alkyl, (C₁-C₆)alkoxy, (C₁-C₆)alkoxy-(C₁-C₆)alkyl-, trifluoromethyl, trifluoromethoxy, cyano, hydroxy, —COOH and —SOₙ(C₁-C₆)alkyl wherein n is zero, one or two, wherein two adjacent ring atoms of ring Y are also ring atoms of ring A; and (c) an optionally substituted (C₁-C₄)heteroalkyl bridge that, together with the atoms to which it is attached, forms a five to seven membered heterocycle containing two to four heteroatoms selected from the group consisting of 1,3-oxazolidin-4-on-5-yl, 1,3-oxazolidin-2,4-dion-5-yl, 4,5-dihydro-1,2-oxazolidin-3-on-4-yl, 1,3-thiazolidin-4-on-5-yl, 1,3-thiazolidin-2,4-dion-5-yl, 1,3-pyrazolidin-4-on-5-yl, 1,3-imidazolidin-2,4-dion-5-yl, 1,2-pyrazolidin-3-on-4-yl, 1,2-thiazolidin-1,1,3-trion-4-yl, 1,2-thiazolidin-3-on-4-yl, tetrahydro-1,2-oxazin-3-on-4-yl, tetrahydro-1,3-oxazin-4-on-5-yl, tetrahydro-1,3-oxazin-2,4-dion-5-yl, morpholin-3-on-2-yl, morpholin-3,5-dion-2-yl, 2,3-dihydro-1,4-oxazin-3-on-2-yl, tetrahydro-1,3-thiazin-4-on-5-yl, tetrahydro-1,3-thiazin-2,4-dion-5-yl, tetrahydro-1,2-thiazin-3-on-4-yl, thiomorpholin-3-on-2-yl, thiomorpholin-3,5-dion-2-yl, 2,3-dihydro-1,4-thiazin-3-on-2-yl, hexahydro-1,2-diazin-3-on-4-yl, 4,5-dihydro-2H-pyridazin-3-on-4-yl, hexahydro-1,3-diazin-4-on-5-yl, hexahydro-1,3-diazin-2,4-dion-5-yl, piperazin-2-on-3-yl, piperazin-2,6-dion-3-yl, tetrahydro-1,3,4-thiadiazin-5-on-6-yl, 5,6-dihydro-1,3,4-thiadiazin-5-on-6-yl, 1,3,4-oxadiazin-5-on-6-yl, 5,6-dihydro-1,2,4-oxadiazin-5-on-6-yl, tetrahydro-1,2,4-oxadiazin-5-on-6-yl, 1,2,4-triazin-5-on-6-yl, tetrahydro-1,2,4-oxadiazin-5-on-6-yl, 5,6-dihydro-1-2,4-oxadiazin-5-on-6-yl, 1,2,4-oxadiazin-3,5-dion-6-yl, 1,2,4-trazin-6-on-5-yl, hexahydro-1,2-oxazepin-3-on-2-yl, hexahydro-1,3-oxazepin-4-on-5-yl, hexahydro-1,4-oxazepin-3-on-2-yl, hexahydro-1,4-oxazepin-3,5-dion-2-yl, hexahydro-1,4-oxazepin-3,5-dion-6-yl, 2,3,5,6-tetrahydro-1-4-oxazepin-5,7-dion-6-yl, hexahydro-1,4-oxazepin-5-on-6-yl, hexahydro-1,3-oxazepin-2,4-dion-5-yl, hexahydro-1,2-thiazepin-3-on-4-yl, hexahydro-1,4-thiazepin-3-on-2-yl, 2,3,4,5-tetrahydro-1,4-thiazepin-3-on-2-yl, hexahydro-1,4-thiazepin-3,5-dion-2-yl, hexahydro-1,4-thiazepin-3,5-dion-6-yl, 2,3,6,7-tetrahydro-1,4-thiazepin-5-on-6-yl, 6,7-dihydro-1,4-thiazepin-5-on-6-yl, hexahydro-1,3-thiazepin-2,4-dion-5-yl, hexahydro-1,2-diazepin-3-on-4-yl, hexahydro-1,3-diazepin-2,4-dion-5-yl, hexahydro-1,4-diazepin-2-on-3-yl, hexahydro-1,4-diazepin-5-on-6-yl, hexahydro-1,4-diazepin-5,7-dion-6-yl, hexahydro-1,3,5-thiadiazepin-3-on-7-yl, 4,5,6,7-tetrahydro-1-3,5-thiadiazepin-6-on-7-yl, and 2,3,5,6-tetrahydro-1,2,4-triazepin-3,5-dion-7-yl; wherein the substituents on any of the carbon atoms capable of supporting an additional bond, of said (C₁-C₄)heteroalkyl bridge, are chloro, fluoro, (C₁-C₆)alkyl, (C₁-C₆)alkoxy, trifluoromethyl or cyano; wherein the substituents on any of the nitrogen atoms capable of supporting an additional bond, of said (C₁-C₄)heteroalkyl bridge, are (C₁-C₆)alkyl or trifluoromethyl,
n2 is one, two, three or four, with the proviso that n2 is one when Y is not CR⁴R⁵;
R⁶ is selected from the group consisting of hydrogen, (C₁-C₆)alkyl optionally substituted with (C₁-C₆)alkoxy or one to three fluorine atoms, or [(C₁-C₄)alkyl]aryl wherein the aryl moiety is phenyl, naphthyl, or heteroaryl-(CH₂)_{q}—, wherein the heteroaryl moiety is selected from the group consisting of pyridyl, pyrimidyl, benzoxazolyl, benzothiazolyl, benzisoxazolyl and benzisothiazolyl and q is zero, one, two, three or four, and wherein said aryl and heteroaryl moieties may optionally be substituted with one or more substituents independently selected from the group consisting of chloro, fluoro, bromo, iodo, (C₁-C₆)alkyl, (C₁-C₆)alkoxy, trifluoromethyl, cyano and —SO_{g}(C₁-C₆)alkyl, wherein g is zero, one or two;
R⁷ is selected from the group consisting of hydrogen, (C₁-C₆)alkyl, [(C₁-C₄)alkyl]aryl wherein the aryl moiety is phenyl, naphthyl, or heteroaryl-(CH₂)ᵣ—, wherein the heteroaryl moiety is selected from the group consisting of pyridyl, pyrimidyl, benzoxazolyl, benzothiazolyl, benzisoxazolyl and benzisothiazolyl and r is zero, one, two, three or four, and wherein said aryl and heteroaryl moieties may optionally be substituted with one or more substituents independently selected from the group consisting of chloro, fluoro, bromo, iodo, (C₁-C₆)alkyl, (C₁-C₆)alkoxy, trifluoromethyl, —C(═O)—(C₁-C₆)alkyl, cyano and —SOⱼ(C₁-C₆)alkyl, wherein j is zero, one or two;
or R⁶ and R⁷ taken together form a C₂-C₄ alkylene chain;
R⁸ is hydrogen or (C₁-C₃)alkyl;
R⁹ is hydrogen or (C₁-C₆)alkyl;
or R⁶ and R⁹, together with the nitrogen atom to which they are attached, form a 5 to 7 membered heteroalkyl ring that contains, in addition to the nitrogen atom to which R⁶ and R⁹ are attached, from zero to four heteroatoms selected from the group consisting of nitrogen, sulfur and oxygen;
and p is one, two, or three;
each of R¹⁰, R¹¹ and R¹² is selected, independently, from the groups set forth in the definition of R²; or R¹¹ and R¹², together with the nitrogen to which they are attached, form a 5 to 7 membered heteroalkyl ring that may contain, in addition to the nitrogen atom to which R¹¹ and R¹² are attached, from zero to four heteroatoms selected from the group consisting of nitrogen, sulfur and oxygen, and
the broken lines indicate optional double bonds, with the proviso that when the broken line in G² is a double bond, R⁸ is absent;
(B)
a compound of the formula II 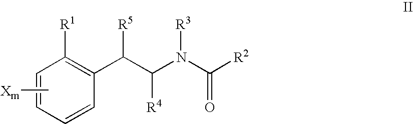
   wherein in Formula II,
R¹ is a group of the formula G¹, G², G³, G⁴, G⁸ or G^{6,} wherein G¹, G², G³, G⁴, and G⁶ are each defined as for formula I, and G⁸ is depicted below 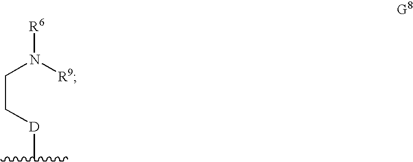
m is 0, 1, 2, 3 or 4;
D is oxygen, sulfur, SO, SO₂, or NR⁷;
a is zero to eight;
p is 1, 2 or 3;
E is oxygen, sulfur, SO or SO₂;
X is hydrogen, chloro, fluoro, bromo, iodo, cyano, (C₁-C₆)alkyl, hydroxy, trifluoromethyl, (C₁-C₆)alkoxy, —S(O)ₜ(C₁-C₆)alkyl wherein t is 0, 1 or 2, —CO₂R¹⁰ or —CONR¹¹R¹²;
R² is —(CH₂)_{y}B, wherein y is 0, 1, 2 or 3, and B is hydrogen, phenyl, naphthyl or a 5 or 6 membered heteroaryl group containing from one to four heteroatoms in the ring, and wherein each of the foregoing phenyl, naphthyl and heteroaryl groups may optionally be substituted with one or more substituents independently selected from chloro, fluoro, bromo, iodo, (C₁-C₆)alkyl, (C₁-C₆)alkoxy, (C₁-C₆)alkoxy-(C₁-C₆)alkyl-, trifluoromethyl, trifluoromethoxy, cyano, hydroxy, —COOH and —SOₙ(C₁-C₆)alkyl wherein n is 0, 1 or 2;
R³ and R⁴ are each independently hydrogen, (C₁-C₄)alkyl or —(CH₂)_{q}-J wherein q is 0, 1, 2 or 3, and J is phenyl or naphthyl, wherein said phenyl or naphthyl may be optionally substituted with one to three substituents independently selected from the group consisting of chloro, fluoro, bromo, iodo, (C₁-C₆)alkyl, (C₁-C₆)alkoxy, trifluoromethyl, cyano and —S(O)ₖ(C₁-C₆)alkyl wherein k is 0, 1 or 2;
R⁵ is hydrogen or (C₁-C₃)alkyl;
R⁶ is selected from the group consisting of hydrogen, (C₁-C₆)alkyl optionally substituted with (C₁-C₆)alkoxy or one to three fluorine atoms, or [(C₁-C₄)alkyl]aryl wherein the aryl moiety is phenyl, naphthyl, or heteroaryl-(CH₂)_{q2}—, wherein the heteroaryl moiety is selected from the group consisting of pyridyl, pyrimidyl, benzoxazolyl, benzothiazolyl, benzisoxazolyl and benzisothiazolyl and q2 is zero, one, two, three or four, and wherein said aryl and heteroaryl moieties may optionally be substituted with one or more substituents independently selected from the group consisting of chloro, fluoro, bromo, iodo, (C₁-C₆)alkyl, (C₁-C₆)alkoxy, trifluoromethyl, cyano and —SO₉(C₁-C₆)alkyl, wherein g is zero, one or two;
R⁷ is selected from the group consisting of hydrogen, (C₁-C₆)alkyl, [(C₁-C₄)alkyl]aryl wherein the aryl moiety is phenyl, naphthyl, or heteroaryl-(CH₂)ᵣ—, wherein the heteroaryl moiety is selected from the group consisting of pyridyl, pyrimidyl, benzoxazolyl, benzothiazolyl, benzisoxazolyl and benzisothiazolyl and r is zero, one, two, three or four, and wherein said aryl and heteroaryl moieties may optionally be substituted with one or more substituents independently selected from the group consisting of chloro, fluoro, bromo, iodo, (C₁-C₆)alkyl, (C₁-C₆)alkoxy, trifluoromethyl, —C(═O)—(C₁-C₆)alkyl, cyano and —SOⱼ(C₁-C₆)alkyl, wherein j is zero, one or two;
or R⁶ and R⁷ taken together form a 2 to 4 carbon chain;
R⁸ is hydrogen or (C₁-C₃)alkyl;
R⁹ is hydrogen or (C₁-C₆)alkyl;
or R⁶ and R⁹, together with the nitrogen atom to which they are attached, form a 5 to 7 membered heteroalkyl ring that contains, in addition to the nitrogen atom to which R⁶ and R⁹ are attached, from zero to four heteroatoms selected from the group consisting of nitrogen, sulfur and oxygen;
each of R¹⁰, R¹¹ and R¹² is selected, independently, from the groups set forth in the definition of R³; or R¹¹ and R¹², together with the nitrogen to which they are attached, form a 5 to 7 membered heteroalkyl ring that may contain, in addition to the nitrogen atom to which R¹¹ and R¹² are attached, from zero to four heteroatoms selected from the group consisting of nitrogen, sulfur and oxygen, and
each R¹³ is, independently, (C₁-C₄)alkyl or a (C₁-C₄)methylene bridge from one of the ring carbons of the piperazine or piperidine ring of G¹ or G², respectively, to the same or another ring carbon or a ring nitrogen of the piperazine or piperidine ring of G¹ or G², respectively, having an available bonding site, or to a ring carbon of R⁶ having an available bonding site;
with the proviso that when B is hydrogen, t is not zero; and
with the proviso that when the broken line in formula G² is a double bond, R⁸ is absent;
and optionally
(iii) a pharmaceutically acceptable carrier.

The present invention also relates to:
a pharmaceutical composition for treating, for example, a disorder or condition that can be treated by enhancing serotonergic neurotransmission in a mammal, preferably a human, comprising components (i), (ii) and optionally (iii) defined herein;
a method for treating a disorder or condition as defined in the previous paragraphs in a mammal, preferably a human, comprising administering to a mammal in need of such treatment components (i) and (ii) as defined herein; and
a method for treating a disorder or condition that can be treated by enhancing serotonergic neurotransmission in a mammal, preferably a human, comprising administering to a mammal in need of such treatment components (i) and (ii) as defined herein.

The 5-HT_{1B} receptor antagonist of the formula I or II defined herein of the compositions and the methods of the invention may be used in an amount that is a serotonin receptor antagonizing or agonizing effective amount.

In the pharmaceutical compositions and methods of the invention, components (i) and (ii) as defined in the previous paragraphs may also be combined with a 5-HT_{1A} antagonist or a pharmaceutically acceptable salt thereof, wherein the amounts of each of components (i), (ii) and the 5-HT_{1A} antagonist or a pharmaceutically acceptable salt thereof are such that the combination of components (i), (ii) and the 5-HT_{1A} antagonist or a pharmaceutically acceptable salt thereof is effective in treating a disorder or condition as defined in the previous paragraphs. For example, the method of the invention may further comprise administering a 5-HT_{1A} antagonist or a pharmaceutically acceptable salt thereof, wherein the amounts of each of components (i), (ii) and the 5-HT_{1A} antagonist or a pharmaceutically acceptable salt thereof are such that the combination of components (i), (ii) and the 5-HT_{1A} antagonist or a pharmaceutically acceptable salt thereof is effective in treating the disorder or condition.

“Enhancing serotonergic neurotransmission,” as used herein, refers to increasing or improving the neuronal process whereby serotonin is released by a pre-synaptic cell upon excitation and crosses the synapse to stimulate or inhibit the post-synaptic cell.

“Chemical dependency,” as used herein, means an abnormal craving or desire for, or an addiction to a drug. Such drugs are generally administered to the affected individual by any of a variety of means of administration, including oral, parenteral, nasal or by inhalation. Examples of chemical dependencies treatable by the methods of the present invention are dependencies on alcohol, nicotine, cocaine, amphetamine and other psychostimulants, morphine, heroin and other opioid agonists, phenobarbital and other barbiturates, and benzodiazepines such as diazepam and others. “Treating a chemical dependency,” as used herein, means reducing or alleviating such dependency.

A “unit dosage form” as used herein is any form that contains a unit dose of the corticotropin releasing factor antagonist or a pharmaceutically acceptable salt thereof, of the compound of formula I or formula II or a pharmaceutically acceptable salt thereof, or of the corticotropin releasing factor antagonist or pharmaceutically acceptable salt thereof and the compound of formula I or formula II or pharmaceutically acceptable salt thereof. A unit dosage form may be, for example, a tablet or a capsule. A unit dose may be an amount which may be predetermined, for example, by a physician.

Examples of the disorders or conditions which may be treated by the methods, compositions and kits of this invention are as follows:
depression, including depression in cancer patients, depression in Parkinson's patients, Postmyocardial Infarction depression, Subsyndromal Symptomatic depression, depression in infertile women, pediatric depression, major depression, single episode depression, recurrent depression, child abuse induced depression, post partum depression, DSM-IV major depression, treatment-refractory major depression, bipolar depression BP I, bipolar depression BP II, depression in patients with human immunodeficiency virus (HIV), severe depression, psychotic depression, post-stroke depression, neuropathic pain, manic depressive illness, including manic depressive illness with mixed episodes and manic depressive illness with depressive episodes, seasonal affective disorder, and major depression with dysthymia.
phobias, including agoraphobia, social phobia and simple phobias;
sexual dysfunction, including premature ejaculation;
eating disorders, including anorexia nervosa and bulimia nervosa;
chemical dependencies, including addictions to alcohol, cocaine, heroin, phenolbarbitol, nicotine and benzodiazepines;
memory disorders, including dementia, amnestic disorders, and age-related cognitive decline (ARCD);
Parkinson's diseases, including dementia in Parkinson's disease, neuroleptic-induced parkinsonism and tardive dyskinesias;
endocrine disorders, including hyperprolactinaemia;
vasospasm, including a vasospasm in the cerebral vasculature;
gastrointestinal tract disorders, including gastrointestinal tract disorders involving changes in motility and secretion;
cancer, including small cell lung carcinoma;
headache, including headache associated with vascular disorders.

Preferred disorders or conditions that may be treated by the methods, compositions and kits of this invention are migraine, depression, obsessive compulsive disorder, post-traumatic stress disorder (PTSD), and eating disorders.

As used herein, “mammal” means any member of the class Mammalia. As an example, the mammal in need of the treatment may be a human. As another example, the mammal in need of the treatment may be a mammal other than a human.

The methods of this invention also encompass treating the diseases or conditions described herein by the co-administration of two separate pharmaceutical compositions. In this latter embodiment, a first composition comprises a CRF antagonist, and a second composition comprises a 5-HT_{1B} receptor antagonist of the formula I or II. These first and second compositions are preferably co-administered either simultaneously, or in a specifically timed manner.

A prodrug of the CRF antagonist, of the 5-HT_{1B} receptor antagonist of the formula I or II, or of both the CRF antagonist and the 5-HT_{1B} receptor antagonist also may be used in the composition and method of the invention. The term “prodrug” refers to compounds that are drug precursors which, following administration, release the drug in vivo via some chemical or physiological process (e.g., a prodrug on being brought to the physiological pH is converted to the desired drug form). A prodrug of any or all of the CRF antagonists or the 5-HT_{1B} receptor antagonists may be used in the methods, kits, and compositions of the instant invention. In general, prodrugs are functional derivatives of these compounds which are readily convertible in vivo. Conventional procedures for the selection and preparation of suitable prodrug derivatives are described, for example, in Design of Prodrugs, ed. H. Bundgaard, Elsevier, 1985 and can be achieved using methods well known to those skilled in the art. All such prodrugs are within the scope of the combinations, pharmaceutical compositions, methods and kits of this invention.

Upon cleavage, exemplary prodrugs release the corresponding free acid (where applicable), and such hydrolyzable ester-forming residues of the prodrugs of this invention include but are not limited to carboxylic acid substituents wherein the free hydrogen is replaced by (C₁-C₄)alkyl, (C₂-C₁₂)alkanoyloxymethyl, (C₄-C₉)1-(alkanoyloxy)ethyl, 1-methyl-1-(alkanoyloxy)-ethyl having from 5 to 10 carbon atoms, alkoxycarbonyloxymethyl having from 3 to 6 carbon atoms, 1-(alkoxycarbonyloxy)ethyl having from 4 to 7 carbon atoms, 1-methyl-1-(alkoxycarbonyloxy)ethyl having from 5 to 8 carbon atoms, N-(alkoxycarbonyl)aminomethyl having from 3 to 9 carbon atoms, 1-(N-(alkoxycarbonyl)amino)ethyl having from 4 to 10 carbon atoms, 3-phthalidyl, 4-crotonolactonyl, gamma-butyrolacton-4-yl, di-N,N-(C₁-C₂)alkylamino(C₂-C₃)alkyl (such as N,N-dimethylaminoethyl), carbamoyl-(C₁-C₂)alkyl, N,N-di(C₁-C₂)-alkylcarbamoyl-(C₁-C₂)alkyl, piperidino-, pyrrolidino-, or morpholino(C₂-C₃)alkyl, and the like.

The present invention also relates to the pharmaceutically acceptable acid addition salts of compounds of the formula I or formula II. The acids which are used to prepare the pharmaceutically acceptable acid addition salts of the aforementioned base compounds of this invention are those which form non-toxic acid addition salts, such as salts containing pharmacologically acceptable anions, such as the hydrochloride, hydrobromide, hydroiodide, nitrate, sulfate, bisulfate, phosphate, acid phosphate, acetate, lactate, citrate, acid citrate, tartrate, bitartrate, succinate, maleate, fumarate, gluconate, saccharate, benzoate, methanesulfonate, ethanesulfonate, benzenesulfonate, p-toluenesulfonate and pamoate [1,1′-methylene-bis-(2-hydroxy-3-naphthoate)]salts.

The invention also relates to base addition salts of formula I or formula II. The chemical bases that may be used as reagents to prepare pharmaceutically acceptable base salts of those compounds of formula I or formula II that are acidic in nature are those that form non-toxic base salts with such compounds. Such non-toxic base salts include, but are not limited to those derived from such pharmacologically acceptable cations such as alkali metal cations, such as potassium and sodium, and alkaline earth metal cations, such as calcium and magnesium, ammonium or water-soluble amine addition salts such as N-methylglucamine-(meglumine), and the lower alkanolammonium and other base salts of pharmaceutically acceptable organic amines.

The compounds of this invention include all stereoisomers, such as cis and trans isomers, and all optical isomers of compounds of the formula I or formula II, such as R and S enantiomers, as well as racemic, diastereomeric and other mixtures of such isomers.

The compounds of this invention may contain C═C double bonds. When such bonds are present, the compounds of the invention exist as cis and trans configurations and as mixtures thereof.

Unless otherwise indicated, the alkyl and alkenyl groups referred to herein, as well as the alkyl moieties of other groups referred to herein, such as alkoxy, may be linear or branched, and they may also be cyclic (e.g., cyclopropyl, cyclobutyl, cyclopentyl, or cyclohexyl) or be linear or branched and contain cyclic moieties. Unless otherwise indicated, halogen includes fluorine, chlorine, bromine, and iodine.

The term “a 5 or 6 membered heteroaryl group containing from one to four heteroatoms in the ring”, as used herein, unless otherwise indicated, includes but is not limited to furyl, thienyl, thiazolyl, pyrazolyl, isothiazolyl, oxazolyl, isoxazolyl, pyrrolyl, triazolyl, tetrazolyl, imidazolyl, 1,3,5-oxadiazolyl, 1,2,4-oxadiazolyl, 1,2,3-oxadiazolyl, 1,3,5-thiadiazolyl, 1,2,3-thiadiazolyl, 1,2,4-thiadiazolyl, pyridyl, pyrimidyl, pyrazinyl, pyridazinyl, 1,2,4-triazinyl, 1,2,3-triazinyl, 1,3,5-triazinyl, benzoxazolyl, benzothiazolyl, benzisothiazolyl, benzisoxazolyl, benzimidazolyl, thianaphthenyl, isothianaphthenyl, benzofuranyl, isobenzofuranyl, isoindolyl, indolyl, indazolyl, isoquinolyl, quinolyl, phthalazinyl, quinoxalinyl, quinazolinyl or benzoxazinyl.

The term “a 5 to 7 membered heteroalkyl ring that may contain from one to four heteroatoms selected from nitrogen, sulfur and oxygen”, as used herein, unless otherwise indicated, includes but is not limited to pyrrolidine, isoxazolidine, 1,3-oxazolidin-3-yl, isothiazolidine, 1,3-thiazolidin-3-yl, 1,2-pyrazolidin-2-yl, 1,3-pyrazolidin-1-yl, piperidine, thiomorpholine, 1,2-tetrahydrothiazin-2-yl, 1,3-tetrahydrothiazin-3-yl, tetrahydrothiadiazine, morpholine, 1,2-tetrahydrodiazin-2-yl, 1,3-tetrahydrodiazin-1-yl, piperazine.

The following are more specific embodiments of groups G¹ and G² of the compound of formula I: 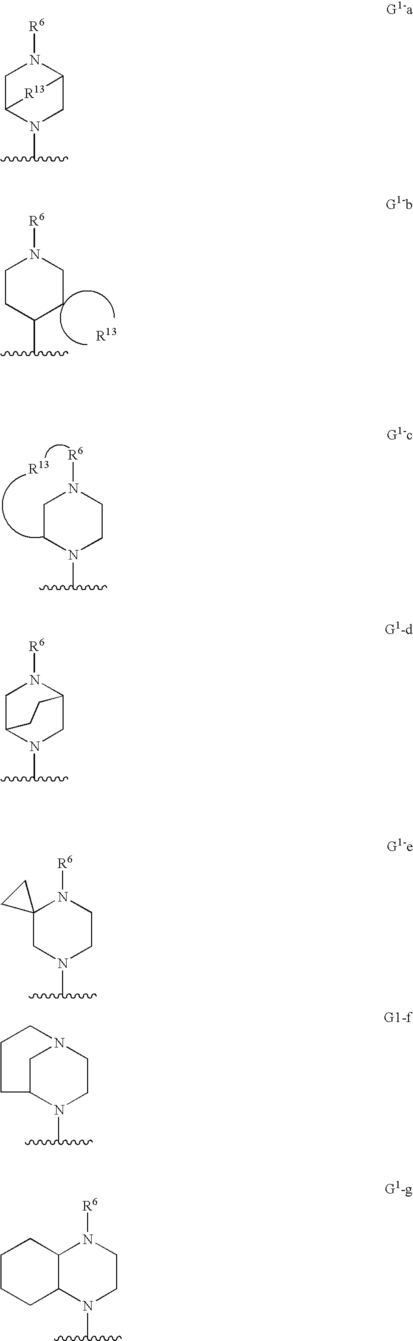 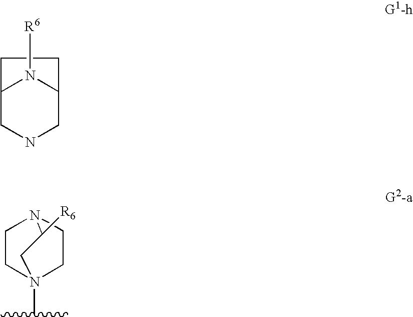
wherein each R¹³ is, independently, (C₁-C₄)alkyl or a (C₁-C₄)methylene bridge from one of the ring carbons of the piperazine or piperidine ring of G¹ or G², respectively, to the same or another ring carbon or a ring nitrogen of the piperazine or piperidine ring of G¹ or G², respectively, having an available bonding site, or to a ring carbon of R⁶ having an available bonding site.

Preferred compounds of the formula I include those wherein R¹ is 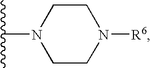
R⁶ is (C₁-C₆)alkyl, such as methyl, and R² is hydrogen.

Other preferred compounds of formula I include those wherein R³ is hydrogen, phenyl or benzyl optionally substituted by chloro, fluoro, bromo, iodo, (C₁-C₆)alkyl or trifluoromethyl.

Other preferred compounds of formula I include those wherein R⁴ is hydrogen or (C₁-C₆)alkyl, such as methyl.

More preferred compounds of formula I include those wherein R¹ is 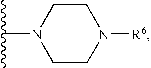
R⁶ is (C₁-C₆)alkyl and R² is hydrogen; R³ is phenyl or benzyl optionally substituted by chloro, fluoro, bromo, iodo, (C₁-C₆)alkyl or trifluoromethyl; and R⁴ is hydrogen or (C₁-C₆)alkyl.

Preferred compounds of the formula I also include those wherein Y, together with the atoms to which it is attached, forms an optionally substituted five to seven membered heterocycle selected from the group consisting of 1,3 thiazolidin-2,4-dion-5-yl, 1,3 imidazolidin-2,4-dion-5-yl, thiomorpholin-3-on-2-yl or morpholin-3-on-2-yl.

Preferred compounds of the formula I also include those wherein R³ is optionally substituted phenyl or —(CH₂)-optionally substituted phenyl, wherein said phenyl groups are optionally substituted with one or more substituents independently selected from the group consisting of chloro, fluoro, bromo, iodo, (C₁-C₆)alkyl, (C₁-C₆)alkoxy, (C₁-C₆)alkoxy-(C₁-C₆)alkyl-, trifluoromethyl, trifluoromethoxy, cyano, hydroxy, —COOH and —SOₙ(C₁-C₆)alkyl wherein n in —SOₙ(C₁-C₆)alkyl is zero, one or two.

Preferred compounds of the formula I also include those wherein R⁵ is hydrogen or methyl.

Preferred compounds of the formula I also include those wherein X is hydrogen, fluoro or chloro, preferably wherein X is hydrogen.

Preferred compounds of the formula I also include those wherein R⁴ and R⁵, together with the nitrogen to which they are attached, form a 5 to 7 membered heteroalkyl ring that is selected from the group consisting of pyrrolidine, isoxazolidine, 1,3-oxazolidin-3-yl, isothiazolidine, 1,3-thiazolidin-3-yl, 1,2-pyrazolidin-2-yl, 1,3-pyrazolidin-1-yl, piperidine, thiomorpholine, 1,2-tetrahydrothiazin-2-yl, 1,3-tetrahydrothiazin-3-yl, tetrahydrothiadiazine, morpholine, 1,2-tetrahydrodiazin-2-yl, 1,3-tetrahydrodiazin-1-yl, and piperazine.

Preferred compounds of the formula I also include those wherein m is 0 or 1.

Preferred compounds of the formula II include those wherein R¹ is 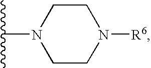
R⁶ is (C₁-C₆)alkyl and R³ is hydrogen.

Other preferred compounds of formula II include those wherein R² is phenyl or benzyl optionally substituted by chloro, fluoro, bromo, iodo, (C₁-C₆)alkyl or trifluoromethyl.

Other preferred compounds of formula II include those wherein R⁴ is hydrogen or (C₁-C₆)alkyl.

More preferred compounds of formula II include those wherein R¹ is 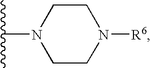
R⁶ is (C₁-C₆)alkyl and R³ is hydrogen; R² is phenyl or benzyl optionally substituted by chloro, fluoro, bromo, iodo, (C₁-C₆)alkyl or trifluoromethyl; and R⁴ is hydrogen or (C₁-C₆)alkyl.

Preferred examples of compounds of component (ii) include:
4-benzyl-2-[2-(4-methylpiperazin-1-yl)-benzylidene]-thiomorpholin-3-one;
4-(3,4-dichlorobenzyl)-2-[2-(4-methylpiperazin-1-yl)-benzylidene]-thiomorpholin-3-one;
2-[2-(4-methylpiperazin-1-yl)-benzylidene]-4-(4-trifluoromethylphenyl)-thiomorpholin-3-one;
2-[2-(4-methylpiperazin-1-yl)-benzylidene]-thiomorpholin-3-one;
4-(3,4-dichlorophenyl)-2-[2-fluoro-6-(4-methylpiperazin-1-yl)-benzylidene]-thiomorpholin-3-one;
4-(3,4-dichlorophenyl)-2-[2-(4-methylpiperazin-1-yl)-benzylidene]-thiomorpholin-3-one;
4-(3,4-dichlorophenyl)-2-[2-(4-methylpiperazin-1-yl)-benzyl]-thiomorpholin-3-one;
4-methyl-2-[2-(4-methylpiperazin-1-yl)-benzylidene]-thiomorpholin-3-one;
4-(3,4-dichlorophenyl)-2-(2-piperazin-1-ylbenzylidene)-thiomorpholin-3-one;
4-(3,4-dichlorophenyl)-2-[2-(4-methyl-(4-piperazin-1-yl)-benzylidene]-1-oxo thiomorpholin-3-one;
4-(3,4-dichlorophenyl)-2-[2-(4-methyl-4-oxy-piperazin-1-yl)-benzylidene]-thiomorpholin-3-one;
10-[4(3,4-dichlorophenyl)-3-oxo-thiomorpholin-2-yl]-2-methyl-3,42-dihydro-pyrazino[1,2-a]indol-2-ium;
4-Benzyl-2-[2-(4-methylpiperazin-1-yl)-benzylidene]-1,1-dioxothiomorpholin-3-one;
4-(3,4-Dichlorophenyl)-2-[3-fluoro-2-(4-methylpiperazin-1-yl)-benzylidene]-thiomorpholin-3-one;
4-(3,4-Dichlorophenyl)-2-[5-fluoro-2-(4-methylpiperazin-1-yl)-benzylidene]-thiomorpholin-3-one;
4-(3,4-Dichlorophenyl)-2-[2-(4-methylpiperazin-1-yl)-5-trifluoromethyl-benzylidene]-thiomorpholin-3-one;
4-(3,4-Dichlorophenyl)-2-{2-[4-(2-methoxyethyl)piperazin-1-yl]-benzylidene}thiomorpholin-3-one;
4-(3,4-Dichlorophenyl)-2-[2-(4-isopropylpiperazin-1-yl)-benzylidene]-thiomorpholin-3-one;
4-(3,4-Dichlorophenyl)-2-[2-(4-ethylpiperazin-1-yl)-benzylidene]-thiomorpholin-3-one;
4-(4-Chlorophenyl)-2-[2-(4-methylpiperazin-1-yl)-benzylidene]-thiomorpholin-3-one;
4-(3-Chlorophenyl)-2-[2-(4-methylpiparazin-1-yl)-benzylidene]-thiomorpholin-3-one;
2-[2-Chloro-6-(4-methylpiperazin-1-yl)-benzylidene]-4-(3,4-dichlorophenyl)-thiomorpholin-3-one;
4-(3,4-Dichlorophenyl)-2-[2-(4-methylpiperazin-1-yl)-4-trifluoromethyl-benzylidene]-thiomorpholin-3-one;
4-(3,4-Dichlorophenyl)-2-[2-(4-methylpiperazin-1-yl)-benzylidene]-1-oxo-thiomorpholin-3-one;
4-(3,4-Dichlorophenyl)-2-(5-fluoro-2-piperazin-1-yl-benzylidene)-thiomorpholin-3-one;
4-(3,4-Dichlorophenyl)-2-[3,6-difluoro-2-(4-methylpiperazin-1-yl)-benzylidene]-thiomorpholin-3-one;
4-(3,4-Dichlorophenyl)-2-[2-(3,5-dimethylpiperazin-1-yl)-benzylidene]-thiomorpholin-3-one;
4-Phenyl-2-[2-(3,4,5-trimethylpiperazin-1-yl)-benzylidene]-thiomorpholin-3-one;
2-[5-Fluoro-2-(4-methylpiperazin-1-yl)-benzylidene]-4-phenyl-thiomorpholin-3-one;
4-Benzo[1,3]dioxol-5-yl-2-[2-(3,5-dimethylpiperazin-1-yl)-benzylidene]-thiomorpholin-3-one;
2-[2-(4-tert-Butylpiperazin-1-yl)-benzylidene]-4-(3,4-dichlorophenyl)-thiomorpholin-3-one;
3-[4-(3,4-Dichlorophenyl)-3-oxo-thiomorpholin-2-ylidenemethyl]-6-dimethylamino-2-(4-methylpiperazin-1-yl)-benzonitrile;
4-(3,4-Dichlorophenyl)-2-[2-(3,4,5-trimethylpiperazin-1-yl)-benzylidene]-thiomorpholin-3-one;
4-(3,4-Dichlorophenyl)-2-[5-methyl-2-(4-methylpiperazin-1-yl)-benzylidene]-thiomorpholin-3-one;
2-[4-Chloro-2-(4-methylpiperazin-1-yl)-benzylidene]-4-(3,4-dichlorophenyl)-thiomorpholin-3-one;
4-(3,4-Difluorophenyl)-2-[2-(3,5-dimethylpiperazin-1-yl)-benzylidene]-thiomorpholin-3-one;
4-(2,4-Difluorophenyl)-2-[2-(3,5-dimethylpiperazin-1-yl)-benzylidene]-thiomorpholin-3-one;
2-(4-Bromo-2-(4-muthylpiperazin-1-yl)-benzylidene]-4-(3,4-dichlorophenyl)-thiomorpholin-3-one;
4-(3,4-Dichlorophenyl)-2-[2-(1-methylpyrrolidin-2-ylmethoxy)-benzylidene]-thiomorpholin-3-one;
4-(3,5-Dichlorophenyl)-2-[2-(3,5-dimethylpiperazin-1-yl)-benzylidene]-thiomorpholin-3-one;
4-(3,4-Difluorophenyl)-2-[2-(3,4,5-trimethylpiperazin-1-yl)-benzylidene]-thiomorpholin-3-one;
4-(3,4-Dichlorophenyl)-2-[2-(octahydropyrido[1,2-a]pyrazin-2-yl)-benzylidene]-thiomorpholin-3-one;
2-[2-(4-Cyclopropylpiperazin-1-yl)-benzylidene]-4-pyridin-3-yl-thiomorpholin-3-one;
2-[2-(4-Cyclopropylpiperazin-1-yl)-benzylidene]-4-(3,4-difluorophenyl)-thiomorpholin-3-one;
2-[2-(4-Cyclopropylpiperazin-1-yl)-benzylidene]-4-(3,5-dichlorophenyl)-thiomorpholin-3-one;
4-(3,4-Difluorophenyl)-2-[2-(2,5-dimethylpiperazin-1-yl)-benzylidene]-thiomorpholin-3-one;
4-(3,5-Dichlorophenyl)-2-[2-(2,5-dimethylpiperazin-1-yl)-benzylidene]-thiomorpholin-3-one;
4-(3,4-Dichlorophenyl)-2-[2-(3-methylaminopyrrolidin-1-yl)-benzylidene]-thiomorpholin-3-one;
4-(3,4-Difluorophenyl)-2-[2-(2,4,5-trimethylpiperazin-1-yl)-benzylidene]-thiomorpholin-3-one;
4-Benzo[1,3]dioxol-5-yl-2-[2-(4-cyclopropylpiperazin-1-yl)-benzylidene]-thiomorpholin-3-one;
2-[2-(3,5-Dimethylpiperazin-1-yl)-benzylidene]-4-(4-fluorophenyl)-thiomorpholin-3-one;
4-Benzo[1,3]dioxol-5-yl-2-[2-(2,5-dimethylpiperazin-1-yl)-benzylidene]-thiomorpholin-3-one;
2-[2-(3,5-Dimethylpiperazin-1-yl)-benzylidene]-4-phenylthiomorpholin-3-one;
4-(3,4-Dichlorophenyl)-2-[2-(4-methylpiperazin-1-yl)-benzylidene]-thiomorpholin-3-one;
4-(3,4-Dichlorophenyl)-2-[2-(3-dimethylaminopyrrolidin-1-yl)-benzylidene]-thiomorpholin-3-one;
4-(3,4-Dichlorophenyl)-2-[2-(3-dimethylaminopyrrolidin-1-yl)-benzylidene]-thiomorpholin-3-one;
4-(3,4-Dichlorophenyl)-2-[2-(4-methyl-[1,4]diazepan-1-yl)-benzylidene]-thiomorpholin-3-one;
4-(3,4-Dichlorophenyl)-2-[2-(2,4,6-trimethylpiperazin-1-yl)-benzylidene]-thiomorpholin-3-one;
2-[2-(4-Cyclopropylpiperazin-1-yl)-benzylidene]-4-(3,4-dichlorophenyl)-thio morpholin-3-one;
   the (−)-enantiomer of a compound of formula 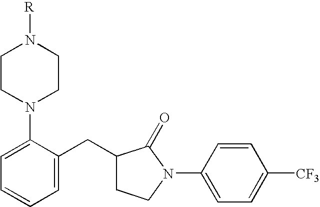
      and pharmaceutically acceptable salts thereof; wherein R is H or CH₃;
   a compound of formula 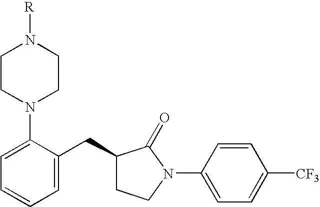
      and pharmaceutically acceptable salts thereof; wherein R is H or CH₃;
(−)-3(S)-[[2-(4-methyl-1-piperazinyl)phenyl]methyl]-1-[4-(trifluoromethyl)phenyl]-2-pyrrolidinone;
   an enantiomeric mixture of (−)-3(S)-[[2-(4-methyl-1-piperazinyl)phenyl]methyl]-1-[4-(trifluoromethyl)phenyl]-2-pyrrolidinone; and (+)-3(R)-[[2-(4-methyl-1-piperazinyl)phenyl]methyl]-1-[4-(trifluoromethyl)phenyl]-2-pyrrolidinone, or pharmaceutically acceptable salts thereof; wherein the ratio of the 3(S)-enantiomer to the (R)-enantiomer is in excess of 2:1, 5:1 or 99:1;
   a compound of formula III 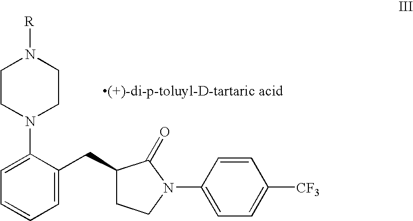
      wherein R is H or CH₃;
3,4-Dichloro-N-{2-[2-(4-methylpiperazin-1-yl)-phenyl]-ethyl}-benzamide;
4-Fluoro-N-{2-[2-(4-methylpiperazin-1-yl)-phenyl]-ethyl}-benzamide;
N-{2-[2-(4-methylpiperazin-1-yl)-phenyl]-ethyl}-benzamide;
3,4-Dichloro-N-(1-methyl-2-[2-(4-methylpiperazin-1-yl)-phenyl]-ethyl)-benzamide;
3,4-Dichloro-N-(1-methyl-2-[2-(4-methylpiperazin-1-yl)-phenyl]-propyl)-benzamide;
3,4-Dichloro-N-methyl-N-{2-[2-(4-methylpiperazin-1-yl)-phenyl]-ethyl}-benzamide;
N-Benzyl-N-(2-[2-(4-methylpiperazin-1-yl)-phenyl]-ethyl)-benzamide;
N-(4-chlorobenzyl)-N-(2-[2-(4-methylpiperazin-1-yl)-phenyl]-ethyl)-benzamide;
3,4-Dichloro-N-2-{2-[methyl-(1-methylpyrolidin-2-ylmethyl)-amino]-phenyl}-ethyl)-benzamide;
3,4-Dichloro-N-{2-[2-(1-methyl-octahydro-pyrrolo[2,3-c]pyridin-6-yl)-phenyl]-ethyl}-benzamide;
3,4-Dichloro-N-{2-[2-(hexahydro-pyrrolo[1,2-a]pyrazin-2-yl)-phenyl]-ethyl}-benzamide;
3,4-Dichloro-N-{2-[2-(1-methylpiperidin-4-yl)-phenyl]-ethyl}-benzamide;
3,4-Dichloro-N-{2-[2-(2-dimethylaminoethoxy)-phenyl]-ethyl}-benzamide;
3,4-Dichloro-N-{2-[2-(2-dimethylamino-ethylsulfanyl)-phenyl]-ethyl}-benzamide;
3,4-Dichloro-N-{2-[2-(2-pyrrolidin-1-ylethoxy)-phenyl]-ethyl}-benzamide;
4-Chloro-N-{2-[2-(3-dimethylamino-pyrrolidin-1-yl)-phenyl]-ethyl}-benzamide;
4-Chloro-N-(2-{2-[methyl-(2-morpholin-4-yl-ethyl)-amino]-phenyl}-ethyl)-benzamide;
2-(4-Chloro-phenyl)-N-{2-[2-(4-methylpiperazin-1-yl)-phenyl]-ethyl}-acetamide;
N-{2-[2-(4-Methylpiperazin-1-yl)-phenyl]-ethyl}-N-phenylacetamide;
N-{2-[2-(4-Methylpiperazin-1-yl)-phenyl]-ethyl}-isonicotinamide;
N-{2-[2-(1-Azabicyclo[2.2.2]oct-4-yl)-phenyl]-ethyl}-N-methylbenzamide;
N-{2-[2-(1,4-Dimethylpiperidin-4-yl)-phenyl]-ethyl}-4-fluorobenzamide;
4-Fluoro-N-{2-[2-(9-methyl-3,9-diazabicyclo[3.3.1]non-3-yl)-phenyl]-ethyl}-benzamide;
N-(2-[2-(1,4-Diazabicyclo[3.3.1]non-4-yl)-phenyl]-ethyl}-N-methylbenzamide;
N-{1-Methyl-2-[2-(5-methyl-2,5-diazabicyclo[2.2.1]hept-2-yl)-phenyl]-ethyl}-benzamide;
2,4-Dichloro-N-methyl-N-{1-methyl-2-[2-(3-methyl-3,8-diazabicyclo[3.2.1]oct-8-yl)-phenyl]-ethyl}-benzamide;
N-{2-[2-(4-Methyl-octahydroquinoxalin-1-yl)-phenyl]-ethyl}-benzamide;
N-{2-[2-(1-Ethylpyrrolidin-2-ylmethoxy)-phenyl]-ethyl}-benzamide;
5-Phenyloxazole-2-carboxylic acid {2-[2-(4-methylpiperazin-1-yl)-phenyl]-ethyl}-amide;
5-Phenylthiophene-2-carboxylic acid {2-[2-(4-methylpiperazin-1-yl)-phenyl]-ethyl}-amide;
5-Methylthiophene-2-carboxylic acid {2-[2-(4-methylpiperazin-1-yl)-phenyl]-ethyl}-amide;
4-Fluoronaphthalene-1-carboxylic acid {2-[2-(4-methylpiperazin-1-yl)-phenyl]-ethyl}-amide;
5-Fluoro-1H-indole-2-carboxylic acid {2-[2-(4-methyl-piperazin-1-yl)-phenyl]-ethyl}-amide;
4-Chloro-N-{2-[2-(3,4,5-trimethylpiperazin-1-yl)-phenyl]-ethyl}-benzamide;
3,4-Dichloro-N-{2-[2-(2,4,5-trimethylpiperazin-1-yl)-phenyl]-ethyl}-benzamide; and
3,4-Dichloro-N-{2-[2-(2,4,6-trimethylpiperazin-1-yl)-phenyl]-ethyl}-benzamide.

Methods for making the 5-HT_{1B} receptor antagonists of the formula I or II described above are disclosed in the above-listed patents and published patent applications incorporated by reference herein, including, for example, U.S. Pat. Nos. 6,462,048; 6,258,953; 6,380,186; and 6,323,229; U.S. Patent Publication Nos. 2002/0091119 and 2003/0083337.

The CRF antagonist may be, for example, a CRF antagonist that has a structure selected from the group of structures IIIa, IIIb and IIIc shown below, and pharmaceutically acceptable salts and esters thereof, as described in WO 95/33750: 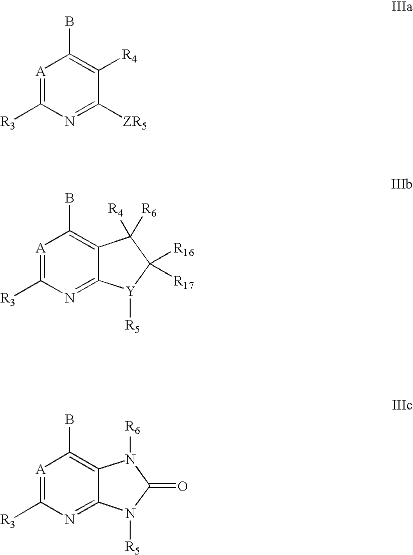
wherein in structures IIIa, IIIb and IIIc
A is CR₇ or N;
B is NR₁R₂, CR₁R₂R₁₁, C(═CR₂R₁₂)R₁, NHCHR₁R₂, OCHR₁R₂, SCHR₁R₂, CHR₂OR₁₂, CHR₂SR₁₂, C(S)R₂ or C(O)R₂;
Y is CH or N;
Z is NH, O, S, N(C₁-C₂ alkyl), or CR₁₃R₁₄, wherein R₁₃ and R₁₄ are each independently hydrogen, trifluoromethyl, or C₁-C₄ alkyl, or one of R₁₃ and R₁₄ may be cyano, chloro, bromo, iodo, fluoro, hydroxy, O(C₁-C₂ alkyl), amino, NH(C₁-C₂ alkyl), or CR₁₃R₁₄ may be C═O or cyclopropyl;
R₁ is C₁-C₆ alkyl which may be substituted by one or two substituents R₈ independently selected from the group consisting of hydroxy, fluoro, chloro, bromo, iodo, C₁-C₄ alkoxy, O—CO—(C₁-C₄ alkyl), O—CO—NH(C₁-C₄ alkyl), O—CO—N(C₁-C₄ alkyl)(C₁-C₂ alkyl), NH(C₁-C₄ alkyl), N(C₁-C₂ alkyl)(C₁-C₄ alkyl), S(C₁-C₄ alkyl), N(C₁-C₄alkyl)CO(C₁-C₄ alkyl), NHCO(C₁-C₄ alkyl), COO(C₁-C₄ alkyl), CONH(C₁-C₄ alkyl), CON(C₁-C₄ alkyl)(C₁-C₂ alkyl), S(C₁-C₄ alkyl), CN, NO₂, SO(C₁-C₄ alkyl), SO₂(C₁-C₄ alkyl), and said C₁-C₆ alkyl or C₁-C₄ alkyl may contain one double or triple bond;
R₂ is C₁-C₁₂ alkyl, aryl or (C₁-C₄ alkylene)aryl wherein said aryl is phenyl, naphthyl, thienyl, benzothienyl, pyridyl, quinolyl, pyrazinyl, pyrimidyl, imidazolyl, furanyl, benzofuranyl, benzothiazolyl, isothiazolyl, benzisothiazolyl, benzisoxazolyl, benzimidazolyl, indolyl, or benzoxazolyl; 3- to 8-membered cycloalkyl or (C₁-C₆ alkylene)cycloalkyl, wherein said cycloalkyl may contain one or two of O, S or N—R₉ wherein R₉ is hydrogen, or C₁-C₄ alkyl, wherein the above defined R₂ may be substituted independently by from one to three of chloro, fluoro, or C₁-C₄ alkyl, or one of bromo, iodo, C₁-C₆ alkoxy, O—CO—(C₁-C₆ alkyl), O—CO—N(C₁-C₄ alkyl)(C₁-C₂ alkyl), S(C₁-C₆ alkyl), CN, NO₂, SO(C₁-C₄ alkyl), or SO₂(C₁-C₄ alkyl), and wherein said C₁-C₁₂ alkyl or C₁-C₄ alkylene may contain one double or triple bond; or
NR₁R₂ or CR₁R₂R₁₁ may form a saturated 5- to 8-membered carbocyclic ring which may contain one or two double bonds or one or two of O or S;
R₃ is methyl, ethyl, fluoro, chloro, bromo, iodo, cyano, methoxy, OCF₃, methylthio, methylsulfonyl, CH₂OH or CH₂OCH₃;
R₄ is hydrogen, C₁-C₄ alkyl, fluoro, chloro, bromo, iodo, C₁-C₄ alkoxy, amino, nitro, NH(C₁-C₄ alkyl), N(C₁-C₄ alkyl)(C₁-C₂ alkyl), SOₙ(C₁-C₄ alkyl), wherein n is 0, 1 or 2, cyano, hydroxy, CO(C₁-C₄ alkyl), CHO, or COO(C₁-C₄ alkyl), wherein said C₁-C₄ alkyl may contain one or two double or triple bonds and may be substituted by one or two of hydroxy, amino, carboxy, NHCOCH₃, NH(C₁-C₂ alkyl), N(C₁-C₂ alkyl)₂, COO(C₁-C₄ alkyl), CO(C₁-C₄ alkyl), C₁-C₃ alkoxy, C₁-C₃ thioalkyl, fluoro, chloro, cyano or nitro;
R₅ is phenyl, naphthyl, thienyl, benzothienyl, pyridyl, quinolyl, pyrazinyl, pyrimidyl, furanyl, benzofuranyl, benzothiazolyl, or indolyl, wherein each one of the above groups R₅ is substituted independently by from one to three of fluoro, chloro, C₁-C₆ alkyl, or C₁-C₆ alkoxy, or one of hydroxy, iodo, bromo, formyl, cyano, nitro, trifluoromethyl, amino, NH(C₁-C₄ alkyl), N(C₁-C₆)(C₁-C₂ alkyl), COOH, COO(C₁-C₄ alkyl), CO(C₁-C₄ alkyl), SO₂NH(C₁-C₄ alkyl), SO₂N(C₁-C₄ alkyl)(C₁-C₂ alkyl), SO₂NH₂, NHSO₂(C₁-C₄ alkyl), S(C₁-C₆ alkyl), or SO₂(C₁-C₆ alkyl), wherein said C₁-C₄ alkyl and C₁-C₆ alkyl may be substituted by one or two of fluoro, hydroxy, amino, methylamino, dimethylamino or acetyl;
R₆ is hydrogen, or C₁-C₆ alkyl, wherein said C₁-C₆ alkyl may be substituted by one hydroxy, methoxy, ethoxy or fluoro;
R₇ is hydrogen, C₁-C₄ alkyl, fluoro, chloro, bromo, iodo, cyano, hydroxy, O(C₁-C₄ alkyl), C(O)(C₁-C₄ alkyl), or C(O)O(C₁-C₄ alkyl), wherein the C₁-C₄ alkyl groups may be substituted with one hydroxy, chloro or bromo, or one to three fluoro;
R₁₁, is hydrogen, hydroxy, fluoro, or methoxy;
R₁₂ is hydrogen or C₁-C₄ alkyl; and
R₁₆ and R₁₇ are each independently hydrogen, hydroxy, methyl, ethyl, methoxy, or ethoxy, except that they are not both methoxy or ethoxy, and CR₄R₆ and CR₁₆R₁₇ each independently may be C═O.

The CRF antagonist can also be of the following structure VI, disclosed in WO 98/05661: 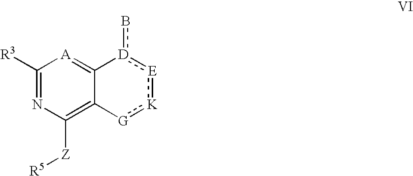
wherein in structure VI the dashed lines represent optional double bonds;
A is nitrogen or CR⁷;
B is —NR¹R², —CR¹R²R¹⁰, —C(═CR²R¹¹)R¹, —NHCR¹R²R¹⁰, —OCR¹R²R¹⁰, —SCR¹R²R¹⁰, —CR²R¹⁰NHR¹, —CR²R¹⁰OR¹, —CR²R¹⁰SR¹ or —COR², and is single bonded to D; or B is —CR¹R², and is double bonded to D and D is carbon;
D is nitrogen or CR⁴ and is single bonded to all atoms to which it is attached, or D is carbon and is double bonded to E or double bonded to B;
E is oxygen, nitrogen, sulfur, C═O, C═S, CR⁶R¹², NR⁶ or CR⁶; or E is a two atom spacer, wherein one of the atoms is oxygen, nitrogen, sulfur, C═O, C═S, CR⁶R¹², NR⁶ or CR⁶, and the other is CR⁶R¹² or CR⁹;
K and G are each, independently, C═O, C═S, sulfur, oxygen, CHR⁸ or NR⁸ when single bonded to both adjacent ring atoms, or nitrogen or CR⁸ when it is double bonded to an adjacent ring atom;
the 6- or 7-membered ring that contains D, E, K and G may contain from one to three double bonds, from zero to two heteroatoms selected from oxygen, nitrogen and sulfur, and from zero to two C═O or C═S groups, wherein the carbon atoms of such groups are part of the ring and the oxygen and sulfur atoms are substituents on the ring;
R¹ is C₁-C₆ alkyl optionally substituted with from one or two substituents independently selected from hydroxy, fluoro, chloro, bromo, iodo, C₁-C₄ alkoxy, CF₃, —C(═O)(C₁-C₄alkyl), —C(═O)—O—(C₁-C₄)alkyl, —OC(═O)(C₁-C₄ alkyl), —OC(═O)N(C₁-C₄ alkyl)(C₁-C₂ alkyl), —NHCO(C₁-C₄ alkyl), —COOH, —COO(C₁-C₄ alkyl), —CONH(C₁-C₄ alkyl), —CON(C₁-C₄ alkyl)(C₁-C₂ alkyl), —S(C₁-C₄ alkyl), —CN, —NO₂, —SO(C₁-C₄ alkyl), —SO₂(C₁-C₄ alkyl), —SO₂NH(C₁-C₄ alkyl) and —SO₂N(C₁-C₄ alkyl)(C₁-C₂ alkyl), wherein each of the C₁-C₄ alkyl groups in the foregoing R¹ groups may optionally contain one or two double or triple bonds;
R² is C₁-C₁₂ alkyl which may optionally contain from one to three double or triple bonds, aryl or (C₁-C₄ alkylene)aryl, wherein said aryl and the aryl moiety of said (C₁-C₄ alkylene)aryl is selected from phenyl, naphthyl, thienyl, benzothienyl, pyridyl, quinolyl, pyrazinyl, pyrimidinyl, imidazolyl, furanyl, benzofuranyl, benzothiazolyl, isothiazolyl, pyrazolyl, pyrrolyl, indolyl, pyrrolopyridyl, oxazolyl and benzoxazolyl; C₃-C₈ cycloalkyl or (C₁-C₆ alkylene)(C₃-C₈ cycloalkyl), wherein one or two of the carbon atoms of said cycloalkyl and the 5 to 8 membered cycloalkyl moieties of said (C₁-C₆ alkylene)(C₃-C₈ cycloalkyl may optionally and independently be replaced by an oxygen or sulfur and wherein each of the foregoing R² groups may optionally be substituted with from one to three substituents independently selected from chloro, fluoro, hydroxy and C₁-C₄ alkyl, or with one substituent selected from C₁-C₆ alkoxy, —OC(═O)(C₁-C₆ alkyl), —OC(═O)N(C₁-C₄ alkyl)(C₁-C₂ alkyl), —S(C₁-C₆ alkyl), amino, —NH(C₁-C₂ alkyl), —N(C₁-C₂ alkyl)(C₁-C₄ alkyl), —N(C₁-C₄ alkyl)-CO—(C₁-C₄ alkyl), —NHCO(C₁-C₄ alkyl), —COOH, —COO(C₁-C₄ alkyl), —CONH(C₁-C₄ alkyl), —CON(C₁-C₄ alkyl)(C₁-C₂ alkyl), —SH, —CN, —NO₂, —SO(C₁-C₄ alkyl), —SO₂(C₁-C₄ alkyl), —SO₂NH(C₁-C₄ alkyl) and —SO₂N(C₁-C₄ alkyl)(C₁-C₂ alkyl);
—NR¹R² or CR¹R²R¹⁰ may form a ring selected from saturated 3 to 8 membered rings, the 5 to 8 membered rings of which may optionally contain one or two double bonds, and wherein one or two of the ring carbon atoms of such 5 to 8 membered rings may optionally and independently be replaced by an oxygen or sulfur atom or by NZ³ wherein Z³ is hydrogen or C₁-C₄ alkyl;
R³ is hydrogen, C₁-C₄ alkyl, —O(C₁-C₄ alkyl), chloro, fluoro, bromo, iodo, —S(C₁-C₄ alkyl) or —SO₂(C₁-C₄ alkyl);
R⁴ is hydrogen, C₁-C₂ alkyl, hydroxy or fluoro;
each R⁶, R⁸ and R⁹ that is attached to a carbon atom is selected, independently, from hydrogen, C₁-C₂ alkyl, fluoro, chloro, bromo, iodo, hydroxy, hydroxymethyl, formyl, trifluoromethyl, cyano, amino, nitro, —O(C₁-C₂ alkyl), —N(C₁-C₂ alkyl)(C₁-C₂ alkyl), —S(C₁-C₂ alkyl), —CO(C₁-C₂ alkyl), —C(═O)H or —C(═O)O(C₁-C₂ alkyl), wherein each of the C₁-C₂ alkyl moieties in the foregoing R⁶, R⁸, and R⁹ groups may optionally contain one double or triple bond; and each R⁶, R⁸, and R⁹ that is attached to a nitrogen atom is selected, independently, from hydrogen and C₁-C₄ alkyl;
R⁵ is substituted phenyl, naphthyl, pyridyl or pyrimidyl, wherein each of the foregoing R⁵ groups is substituted with from two to four substituents R¹⁵, wherein from one to three of said substituents may be selected, independently, from chloro, C₁-C₆ alkyl, —O(C₁-C₆ alkyl) and —(C₁-C₆alkylene)O(C₁-C₆alkyl), and wherein one of said substituents may be selected, independently, from bromo, iodo, formyl, cyano, trifluoromethyl, nitro, amino, —NH(C₁-C₄ alkyl), —N(C₁-C₂ alkyl)(C₁-C₆ alkyl), —C(═O)O(C₁-C₄ alkyl), —C(═O)(C₁-C₄ alkyl), —COOH, —SO₂NH(C₁-C₄ alkyl), —SO₂N(C₁-C₂ alkyl)(C₁-C₄ alkyl), —SO₂NH₂, —NHSO₂(C₁-C₄ alkyl), —S(C₁-C₆ alkyl) and —SO₂(C₁-C₆ alkyl), and wherein each of the C₁-C₄ alkyl and C₁-C₆ alkyl moieties in the foregoing R⁵ groups may optionally be substituted with one or two substituents independently selected from fluoro, hydroxy, amino, methylamino, dimethylamino and acetyl;
R⁷ is hydrogen, methyl, halo, hydroxy, methoxy, —C(═O)(C₁-C₂ alkyl), —C(═O)O(C₁-C₂ alkyl), trifluoromethoxy, hydroxymethyl, trifluoromethyl or formyl;
R¹⁰ is hydrogen, hydroxy, methoxy or fluoro;
R¹¹ is hydrogen or C₁-C₄ alkyl;
R¹² is, hydrogen or methyl; and
Z is NH, oxygen, sulfur, —N(C₁-C₄ alkyl), or CR¹³R¹⁴ wherein R¹³ and R¹⁴ are independently selected from hydrogen, and methyl with the exception that one of R¹³ and R¹⁴ may optionally be cyano;
with the proviso that: (a) in the six or seven membered rings of structures in formula I, there can not be two double bonds adjacent to each other; and (b) when D is carbon and is double bonded to B, then B is CR¹R²;
or a pharmaceutically acceptable salt of such compound.

Other useful CRF antagonists are of the following structure VIII, disclosed in WO 98/08846: 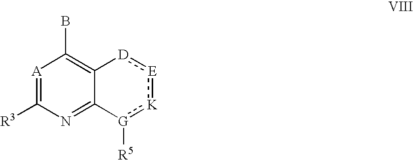
wherein in structure VIII the dashed lines represent optional double bonds;
A is nitrogen or CR⁷;
B is —NR¹R², —CR¹R²R¹⁰, —C(═CR²R¹¹)R¹, —NHCR¹R²R¹⁰, —OCR¹R²R¹⁰, —SCR¹R²R¹⁰, —CR²R¹⁰NHR¹, —CR²R¹⁰OR¹, —C₂R¹⁰SR¹ or —COR²;
G is nitrogen or CR⁴ and is single bonded to all atoms to which it is attached, or G is carbon and is double bonded to K;
K is nitrogen or CR⁶ when double bonded to G or E, or K is oxygen, sulfur, C═O, C═S, CR⁶R¹² or NR⁸ when single bonded to both adjacent ring atoms, or K is a two atom spacer, wherein one of the two ring atoms of the spacer is oxygen, nitrogen, sulfur, C═O, C═S, CR⁶R¹², NR⁶ or CR⁶, and the other is CR⁶R¹² or CR⁹;
D and E are each, independently, C═O, C═S, sulfur, oxygen, CR⁴R⁶ or NR⁸ when single bonded to both adjacent ring atoms, or nitrogen or CR⁴ when it is double bonded to an adjacent ring atom;
the 6- or 7-membered ring that contains D, E, K and G may contain from one to three double bonds, from zero to two heteroatoms selected from oxygen, nitrogen and sulfur, and from zero to two C═O or C═S groups, wherein the carbon atoms of such groups are part of the ring and the oxygen and sulfur atoms are substituents on the ring;
R¹ is C₁-C₆ alkyl optionally substituted with from one or two substituents independently selected from hydroxy, fluoro, chloro, bromo, iodo, C₁-C₄ alkoxy, CF₃, —C(═O)(C₁-C₄alkyl), —C(═O)—O—(C₁-C₄)alkyl, —OC(═O)(C₁-C₄ alkyl), —OC(═O)N(C₁-C₄ alkyl)(C₁-C₂ alkyl), —NHCO(C₁-C₄ alkyl), —COOH, —COO(C₁-C₄ alkyl), —CONH(C₁-C₄ alkyl), —CON(C₁-C₄ alkyl)(C₁-C₂ alkyl), —S(C₁-C₄ alkyl), —CN, —NO₂, —SO(C₁-C₄ alkyl), —SO₂(C₁-C₄ alkyl), —SO₂NH(C₁-C₄ alkyl) and —SO₂N(C₁-C₄ alkyl)(C₁-C₂ alkyl), wherein each of the C₁-C₄ alkyl groups in the foregoing R¹ groups may optionally contain one or two double or triple bonds;
R² is C₁-C₁₂ alkyl which may optionally contain from one to three double or triple bonds, aryl or (C₁-C₄ alkylene)aryl, wherein said aryl and the aryl moiety of said (C₁-C₄ alkylene)aryl is selected from phenyl, naphthyl, thienyl, benzothienyl, pyridyl, quinolyl, pyrazinyl, pyrimidinyl, imidazolyl, furanyl, benzofuranyl, benzothiazolyl, isothiazolyl, pyrazolyl, pyrrolyl, indolyl, pyrrolopyridyl, oxazolyl and benzoxazolyl; C₃-C₈ cycloalkyl or (C₁-C₆ alkylene)(C₃-C₈ cycloalkyl), wherein one or two of the carbon atoms of said cycloalkyl and the 5 to 8 membered cycloalkyl moieties of said (C₁-C₆ alkylene)(C₃-C₈ cycloalkyl may optionally and independently be replaced by an oxygen or sulfur atom or by NZ wherein Z is hydrogen, C₁-C₄ alkyl or benzyl, and wherein each of the foregoing R² groups may optionally be substituted with from one to three substituents independently selected from chloro, fluoro, hydroxy and C₁-C₄ alkyl, or with one substituent selected from C₁-C₆ alkoxy, —OC(═O)(C₁-C₆ alkyl), —OC(═O)N(C₁-C₄ alkyl)(C₁-C₂ alkyl), —S(C₁-C₆ alkyl), amino, —NH(C₁-C₂ alkyl), —N(C₁-C₂ alkyl)(C₁-C₄ alkyl), —N(C₁-C₄ alkyl)-CO—(C₁-C₄ alkyl), —NHCO(C₁-C₄ alkyl), —COOH, —COO(C₁-C₄ alkyl), —CONH(C₁-C₄ alkyl), —CON(C₁-C₄ alkyl)(C₁-C₂ alkyl), —SH, —CN, —NO₂, —SO(C₁-C₄ alkyl), —SO₂(C₁-C₄ alkyl), —SO₂NH(C₁-C₄ alkyl) and —SO₂N(C₁-C₄ alkyl)(C₁-C₂ alkyl);
—NR¹R² or CR¹R²R¹⁰ may form a ring selected from saturated 3 to 8 membered rings, the 5 to 8 membered rings of which may optionally contain one or two double bonds, and wherein one or two of the ring carbon atoms of such 5 to 8 membered rings may optionally and independently be replaced by an oxygen or sulfur atom or by NZ² wherein Z² is hydrogen, benzyl or C₁-C₄ alkyl;
R³ is hydrogen, C₁-C₄ alkyl, —O(C₁-C₄ alkyl), chloro, fluoro, bromo, iodo, —S(C₁-C₄ alkyl) or —SO₂(C₁-C₄ alkyl);
each R⁸, R⁹ and R¹² is selected, independently, from hydrogen and C₁-C₂ alkyl;
each R⁴ and R⁶ that is attached to a carbon atom is selected, independently, from hydrogen and C₁-C₆ alkyl, fluoro, chloro, bromo, iodo, hydroxy, hydroxy(C₁-C₂ alkyl), trifluoromethyl, cyano, amino, nitro, —O(C₁-C₄ alkyl), —N(C₁-C₄ alkyl)(C₁-C₂ alkyl), —CH₂SCH₃, —S(C₁-C₄ alkyl), —CO(C₁-C₄ alkyl), —C(═O)H or —C(═O)O(C₁-C₄ alkyl), wherein each of the C₁-C₂ alkyl moieties in the foregoing R⁴ and R⁶ groups may optionally contain one double or triple bond; and R⁶, when attached to a nitrogen atom, is selected from hydrogen and C₁-C₄ alkyl;
R⁵ is substituted phenyl, naphthyl, pyridyl or pyrimidyl, wherein each of the foregoing R⁵ groups is substituted with from two to four substituents R¹³, wherein up to three of said substituents may be selected, independently, from chloro, C₁-C₆ alkyl, —O(C₁-C₆ alkyl) and —(C₁-C₆ alkylene)O(C₁-C₆alkyl), and wherein one of said substituents may be selected, independently, from bromo, iodo, formyl, cyano, trifluoromethyl, nitro, amino, —NH(C₁-C₄ alkyl), —N(C₁-C₂ alkyl)(C₁-C₆ alkyl), —C(═O)O(C₁-C₄ alkyl), —C(═O)(C₁-C₄ alkyl), —COOH, —SO₂NH(C₁-C₄ alkyl), —SO₂N(C₁-C₂ alkyl)(C₁-C₄ alkyl), —SO₂NH₂, —NHSO₂(C₁-C₄ alkyl), —(C₀-C₁alkylene)-S—(C₁-C₂alkyl), —(C₀-C₁alkylene)-SO—(C₁-C₂alkyl), —(C₀-C₁alkylene)-SO₂-(C₁-C₂alkyl) and —(C₁-C₄alkylene)-OH, and wherein each of the C₁-C₄ alkyl and C₁-C₆ alkyl moieties in the foregoing R⁵ groups may optionally be substituted with one or two substituents independently selected from fluoro, hydroxy, amino, methylamino, dimethylamino and acetyl;
R⁷ is hydrogen, methyl, halo (e.g., chloro, fluoro, iodo or bromo), hydroxy, methoxy, —C(═O)(C₁-C₂ alkyl), —C(═O)O(C₁-C₂ alkyl), hydroxymethyl, trifluoromethyl or formyl;
R¹⁰ is hydrogen, hydroxy, methoxy or fluoro; and
R¹¹ is hydrogen or C₁-C₄ alkyl;
with the proviso that in the ring containing D, E, K and G of formula I, there can not be two double bonds adjacent to each other;
and the pharmaceutically acceptable salt of such compound.

The CRF antagonist may also be of the following structure IX, disclosed in WO 95/10506: 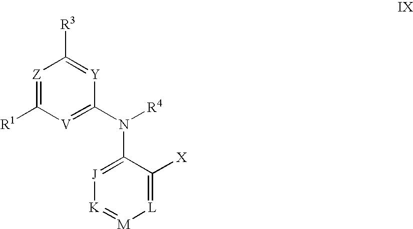
or a pharmaceutically, acceptable salt or prodrug thereof, wherein in structure IX
Y is CR^{3a}, N, or CR²⁹;
when Y is CR^{3a} or N:
R¹ is independently selected at each occurrence from the group consisting of C₁-C₄ alkyl, C₂-C₄ alkenyl, C₂-C₄ alkynyl, halogen, C₁-C₂ haloalkyl, NR⁶R⁷, OR⁸, and S(O)ₙR⁸; R³ is C₁-C₄ alkyl, aryl, C₃-C₆ cycloalkyl, C₁-C₂ haloalkyl, halogen, nitro, NR⁶R⁷, OR⁸, S(O)ₙR⁸C(═O)R⁹, C(═O)NR⁶R⁷, C(═S)NR⁶R⁷, —(CHR¹⁶)ₖNR⁶R⁷, (CH₂)ₖOR⁸, C(═O)NR¹⁰CH(R¹¹)CO₂R¹², C(OH)(R²⁵)(R^{25a}), —(CH₂)ₚS(O)ₙ-alkyl, —(CHR¹⁶)R²⁵, —C(CN)(R²⁵)(R¹⁶) provided that R²⁵ is not —NH— containing rings, —C(═O)R²⁵, —CH(CO₂R¹⁶)₂, NR¹⁰C(═O)CH(R¹¹)NR¹⁰R¹², NR¹⁰CH(R¹¹)CO₂R¹²; substituted C₁-C₄ alkyl, substituted C₂-C₄ alkenyl, substituted C₂-C₄ alkynyl, substituted C₁-C₄ alkoxy, aryl-(substituted C₁-C₄)alkyl, aryl-(substituted C₁-C₄)alkoxy, substituted C₃-C₆ cycloalkyl, amino-(substituted C₁-C₄)alkyl, substituted C₁-C₄ alkylamino, where substitution by R²⁷ can occur on any carbon containing substituent; 2-pyridinyl, imidazolyl, 3-pyridinyl, 4-pyridinyl, 2-methyl-3-pyridinyl, 4-methyl-3-pyridinyl, furanyl, 5-methyl-2-furanyl, 2,5-dimethyl-3-furanyl, 2-thienyl, 3-thienyl, 5-methyl-2-thienyl, 2-pheno-thiazinyl, 4-pyrazinyl, azetidinyl, phenyl, 1H-indazolyl, 2-pyrrolidonyl, 2H,6H-1,5,2-dithiazinyl, 2H-pyrrolyl, 3H-indolyl, 4-piperidonyl, 4aH-carbazolyl, 4H-quinolizinyl, 6H-1,2,5-thiadiazinyl, acridinyl, azocinyl, azepinyl, benzofuranyl, benzothiophenyl, carbazolyl, chromanyl, chromenyl, cinnolinyl, decahydroquinolinyl, furazanyl, imidazolidinyl, indolinyl, indolizinyl, indolyl, isobenzofuranyl, isochromanyl, isoindolinyl, isoindolyl, isoquinolinyl, benzimidazolyl, isothiazolyl, isoxazolyl, morpholinyl, naphthyridinyl, octahydroisoquinolinyl, oxazolidinyl, oxazolyl, phenanthridinyl, phenanthrolinyl, phenazinyl, phenoxathiinyl, phenoxazinyl, phthalazinyl, piperazinyl, piperidinyl, pteridinyl, purinyl, pyranyl, pyrazolidinyl, pyrazolinyl, pyrazolyl, pyridazinyl, pyrimidinyl, pyrrolidinyl, pyrrolinyl, pyrrolyl, quinazolinyl, quinolinyl, quinoxalinyl, quinuclidinyl, β-carbolinyl, tetrahydrofuranyl, tetrahydroisoquinolinyl, tetrahydroquinolinyl, tetrazolyl, thianthrenyl, thiazolyl, thiophenyl, triazinyl, xanthenyl; or 1-tetrahydroquinolinyl or 2-tetrahydroisoquinolinyl either of which can be substituted with 0-3 groups chosen from keto and C₁-C₄ alkyl; J, K, and L are independently selected at each occurrence from the group of N, CH, and CX′;
M is CR⁵ or N;
V is CR^{1a} or N;
Z is CR² or N;
R^{1a}, R², and R^{3a} are independently selected at each occurrence from the group consisting of hydrogen, halo, halomethyl, C₁-C₃ alkyl, and cyano;
R⁴ is (CH₂)ₘOR¹⁶, C₁-C₄ alkyl, allyl, propargyl, (CH₂)ₘR¹³, or —(CH₂)ₘOC(O)R¹⁶;
X is halogen, aryl, heteroaryl, S(O)₂R⁸, SR⁸, halomethyl, —(CH₂)ₚOR⁸, cyano, —(CHR¹⁶)ₚNR¹⁴R¹⁵, —C(═O)R⁸, C₁-C₆ alkyl, C₄-C₁₀ cycloalkylalkyl, C₁-C₁₀alkenyl, C₂-C₁₀alkynyl, C₂-C₁₀alkoxy, aryl-(C₂-C₁₀)-alkyl, C₃-C₆cycloalkyl, aryl-(C₁-C₁₀)-alkoxy, nitro, thio-(C₁-C₁₀)-alkyl, —C(═NOR¹⁶)—C₁-C₄-alkyl, —C(═NOR¹⁶)H, or —C(═O)NR¹⁴R¹⁵, where substitution by R¹⁸ can occur on any carbon containing substituents;
X′ is independently selected at each occurrence from the group consisting of hydrogen, halogen, aryl, heteroaryl, S(O)NR⁸, halomethyl, —(CHR¹⁶)ₚOR⁸, cyano, —(CHR¹⁶)ₚNR¹⁴R¹⁵, C(═O)R⁸, C₁-C₆ alkyl, C₂-C₁₀alkenyl, C₂-C₁₀alkynyl, C₁-C₁₀alkoxy, aryl-(C₁-C₁₀)-alkyl, C₃-C₆cycloalkyl, aryl-(C₁-C₁₀)-alkoxy, nitro, thio-(C₁-C₁₀)-alkyl, —C(═NOR¹⁶)—C₁-C₄-alkyl, —C(═NOR¹⁶)H, and —C(═O)NR¹⁴R¹⁵, where substitution by R¹⁶ can occur on any carbon containing substituents;
R⁵ is halo, —C(═NOR¹⁶)—C₁-C₄-alkyl, C₁-C₄alkyl, C₁-C₃ haloalkyl, —(CHR¹⁶)ₚOR⁸, —(CHR¹⁶)ₚS(O)ₙR⁸, —(CHR⁶)ₚNR¹⁴R¹⁵, C₃-C₆ cycloalkyl, C₂-C₁₀alkenyl, C₂-C₁₀alkynyl, aryl-(C₂-C₁₀)-alkyl, aryl-(C₁-C₁₀)-alkoxy, cyano, C₃-C₆ cycloalkoxy, nitro, amino-(C₂-C₁₀)-alkyl, thio-(C₂-C₁₀)-alkyl, SON(R⁸), C(═O)R⁸—C(═NOR¹⁶)H, or —C(═O)NR¹⁴R¹⁵, where substitution by R¹⁸ can occur on any carbon containing substituents;
R⁶ and R⁷ are independently selected at each occurrence from the group consisting of hydrogen, C₁-C₆ alkyl, C₃-C₁₀ cycloalkyl, C₁-C₆ alkoxy, (C₄-C₁₂)-cycloalkylalkyl, —(CH₂)ₖR¹³, (CHR¹⁶)ₚOR⁸, —(C₁-C₆alkyl)-aryl, heteroaryl, —S(O)_{z}-aryl or —(C₁-C₆alkyl)-heteroaryl or aryl, wherein the aryl or heteroaryl groups are optionally substituted with 1-3 groups selected from the group consisting of hydrogen, halogen, C₁-C₆ alkyl, C₁-C₆ alkoxy, amino, NHC(═O)(C₁-C₆ alkyl), NH(C₁-C₆ alkyl), N(C₁-C₆ alkyl)₂, nitro, carboxy, CO₂(C₁-C₆ alkyl), cyano, and S(O)₂—(C₁-C₆-alkyl); or can be taken together to form —(CH₂)ₚA(CH₂)ᵣ—, optionally substituted with 0-3 R¹⁷; or, when considered with the commonly attached nitrogen, can be taken together to form a heterocycle, said heterocycle being substituted on carbon with 1-3 groups consisting of hydrogen, C₁-C₆ alkyl, hydroxy, or C₁-C₆ alkoxy;
A is CH₂, O, NR²⁵, C(═O), S(O)ₙ, N(C(═O)R¹⁷), N(R¹⁹), C(H)(NR¹⁴R¹⁵), C(H)(OR²⁰), C(H)(C(═O)R²¹), or N(S(O)ₙR²¹);
R⁸ is independently selected at each occurrence from the group consisting of hydrogen; C₁-C₆ alkyl; —(C₄-C₁₂)cycloalkylalkyl; (CH₂)ₜR²²; C₃-C₁₀ cycloalkyl; —NR⁶R⁷; aryl; heteroaryl; —NR¹⁶(CH₂)ₙR⁶R⁷; —(CH₂)ₖR²⁵; and (CH₂)ₜheteroaryl or (CH₂)ₜaryl, either of which can optionally be substituted with 1-3 groups selected from the group consisting of hydrogen, halogen, C₁-C₆ alkyl, C₁-C₆ alkoxy, amino, NHC(═O)(C₁-C₆ alkyl), NH(C₁-C₆ alkyl), N(C₁-C₆ alkyl)₂, nitro, carboxy, CO₂(C₁-C₆ alkyl), cyano, and S(O)₂(C₁-C₆-alkyl);
R⁹ is independently selected at each occurrence from R¹⁰, hydroxy, C₁-C₄ alkoxy, C₃-C₆ cycloalkyl, C₂-C₄ alkenyl, aryl substituted with 0-3 R¹⁸, and —(C₁-C₆ alkyl)-aryl substituted with 0-3 R¹⁸;
R¹⁰, R¹⁶, R²⁴, and R² are independently selected at each occurrence from hydrogen or C₁-C₄ alkyl;
R¹¹ is C₁-C₄ alkyl substituted with 0-3 groups chosen from the following: keto, amino, sulfhydryl, hydroxyl, guanidinyl, p-hydroxyphenyl, imidazolyl, phenyl, indolyl, and indolinyl, or, when taken together with an adjacent R¹⁰, are (CH₂)ₜ;
R¹² is hydrogen or an appropriate amine protecting group for nitrogen or an appropriate carboxylic acid protecting group for carboxyl;
R¹³ is independently selected at each occurrence from the group consisting of CN, OR¹⁹, SR¹⁹, and C₃-C₆ cycloalkyl;
R¹⁴ and R¹⁵ are independently selected at each occurrence from the group consisting of hydrogen, C₄-C₁₀, cycloalkyl-alkyl, and R¹⁰;
R¹⁷ is independently selected at each occurrence from the group consisting of R¹⁰, C₁-C₄ alkoxy, halo, OR²³, SR²³, NR²⁴, and (C₁-C₆)alkyl (C₁-C₄)alkoxy;
R₁₈ is independently selected at each occurrence from the group consisting of R¹⁰, hydroxy, halogen, C₁-C₂ haloalkyl, C₁-C₄ alkoxy, C(═O)R²⁴, and cyano;
R¹⁹ is independently selected at each occurrence from the group consisting of C₁-C₆ alkyl, C₃-C₆ cycloalkyl, (CH₂)R²², and aryl substituted with 0-3 R¹⁰;
R²⁰ is independently selected at each occurrence from the group consisting of R¹⁰, C(═O)R³¹, and C₂-C₄ alkenyl;
R²¹ is independently selected at each occurrence from the group consisting of R¹⁰, C₁-C₄ alkoxy, NR²³R²⁴, and hydroxyl;
R²² is independently selected at each occurrence from the group consisting of cyano, OR²⁴, SR²⁴, NR²³R²⁴, C₁-C₆ alkyl, C₃-C₆ cycloalkyl, —S(O)ₙR³¹, and —C(═O)R²⁵;
R²⁵, which can be optionally substituted with 0-3 R17, is independently selected at each occurrence from the group consisting of phenyl, pyrazolyl, imidazolyl, 2-methyl-3-pyridinyl, 4-methyl-3-pyridinyl, furanyl, 5-methyl-2-furanyl, 2,5-dimethyl-3-furanyl, 2-thienyl, 3-thienyl, 5-methyl-2-thienyl, 2-pheno-thiazinyl, 4-pyrazinyl, azetidinyl, 1H-indazolyl, 2-pyrrolidonyl, 2H,6H-1,5,2-dithiazinyl, 2H-pyrrolyl, 3H-indolyl, 4-piperidonyl, 4aH-carbazolyl, 4H-quinolizinyl, 6H-1,2,5-thiadiazinyl, acridinyl, azocinyl, azepinyl, benzofuranyl, benzothiophenyl, carbazolyl, chromanyl, chromenyl, cinnolinyl, decahydroquinolinyl, furazanyl, indolinyl, indolizinyl, indolyl, isobenzofuranyl, isochromanyl, isoindolinyl, isoindolyl, isoquinolinyl benzimidazolyl, isothiazolyl, isoxazolyl, morpholinyl, naphthyridinyl, octahydroisoquinolinyl, oxazolidinyl, oxazolyl, phenanthridinyl, phenanthrolinyl, phenazinyl, phenothiazinyl, phenoxathiinyl, phenoxazinyl, phthalazinyl, piperazinyl, piperidinyl, pteridinyl, purinyl, pyranyl, pyrazolidinyl, pyridazinyl, pyridyl, pyrimidinyl, pyrrolidinyl, pyrrolinyl, pyrrolyl, quinazolinyl, quinolinyl, quinoxalinyl, quinuclidinyl, B-carbolinyl, tetrahydrofuranyl, tetrazolyl, thianthrenyl, thiazolyl, thiophenyl, triazinyl, xanthenyl; and 1-tetrahydroquinolinyl or 2-tetrahydroisoquinolinyl either of which can be substituted with 0-3 groups chosen from keto and C₁-C₄ alkyl;
R^{25a}, which can be optionally substituted with 0-3 R¹⁷, is independently selected at each occurrence from the group consisting of H and R²⁵;
R²⁷ is independently selected at each occurrence from the group consisting of C₁-C₃ alkyl, C₂-C₄ alkenyl, C₂-C₄ alkynyl, C₂-C₄ alkoxy, aryl, nitro, cyano, halogen, aryloxy, and heterocycle optionally linked through 0;
R³¹ is independently selected at each occurrence from the group consisting of C₁-C₄ alkyl, C₃-C₇ cycloalkyl, C₄-C₁₀ cycloalkyl-alkyl, and aryl-(C₁-C₄)alkyl;
k, m, and r are independently selected at each occurrence from 1-4;
n is independently, selected at each occurrence from 0-2,
p, q, and z are independently selected at each occurrence from 0-3;
t and w are independently selected at each occurrence from 1-6, provided that when J is CX′ and K and L are both CH, and M is CR⁵, then
   (A) when V and Y are N and Z is CH and R¹ and R³ are methyl,
      (1) and R⁴ is methyl, then
         (a) R⁵ can not be methyl when X is OH and X′ is H;
         (b) R⁵ can not be —NHCH₃, or —N(CH₃)₂ when X and X′ are —OCH₃; and
         (c) R⁵ can not be —N(CH₃)₂ when X and X′ are —OCH₂CH₃;
      (2) and R⁴ is ethyl, then
         (a) R⁵ can not be methylamine when X and X′ are —OCH₃;
         (b) R⁵ can not be OH when X is Br and X′ is OH; and
         (c) R⁵ can not be —CH₂OH or —CH₂N(CH₃)₂ when X is —SCH₃ and X′ is H;
   (B) when V and Y are N, Z is CH, R⁴ is ethyl, R⁵ is iso-propyl, X is Br, X′ is H, and
      (1) R¹ is CH₃, then
         (a) R³ can not be OH, piperazin-1-yl, —CH_{2,}-piperidin-1-yl, —CH₂—(N-4-methylpiperazin-1-yl), —C(O)NH-phenyl, —CO₂H, —CH₂O-(4-pyridyl), —C(O)NH₂, 2-indolyl, —CH₂O-(4-carboxyphenyl), —N(CH₂CH₃)(2-bromo-4-isopropylphenyl);
      (2) R² is —CH₂CH₂CH₃ then R³ can not be —CH₂CH₂CH₃
   (C) when V, Y and Z are N, R⁴ is ethyl, and
      (1) R⁵ is iso-propyl, X is bromo, and X′ is H, then
         (a) R³ can not be OH or —OCH₂CN when R¹ is CH₃ and
         (b) R³ can not be —N(CH₃)₂ when R¹ is —N(CH₃)₂;
      (2) R⁵ is —OCH₃, X is —OCH₃, and X′ is H, then R³ and R¹ can not both be chloro; further provided that when J, K, and L are all CH and M is CR⁵, then
   (D) at least one of V, Y, and Z must be N;
   (E) when V is CR^{1a}, Z and Y can not both be N;
   (F) when Y is CR^{3a}, Z and V can not both be N;
   (G) when Z is CR², V and Y must both be N;
   (H) Z can be N only when both V and Y are N or when V is CR^{1a} and Y is CR^{3a};
   (I) when V and Y are N, Z is CR², and R² is H or C₁-C₃ alkyl, and R⁴ is C₁-C₃ alkyl, R³ can not be 2-pyridinyl, indolyl, indolinyl, imidazolyl, 3-pyridinyl, 4-pyridinyl, 2-methyl-3-pyridinyl, 4-methyl-3-pyridinyl, furanyl, 5-methyl-2-furanyl, 2,5-dimethyl-3-furanyl, 2-thienyl, 3-thienyl, 5-methyl-2-thienyl, 2-phenothiazinyl, or 4-pyrazinyl;
   (J) when V and Y are N; Z is CR²; R² is H or C₁-C₃ alkyl; R⁴ is C₁-C₄ alkyl, R⁵, X, and/or X′ are OH, halo, CF₃, C₁-C₄ alkyl, C₁-C₄ alkoxy, C₁-C₄ alkylthio, cyano, amino, carbamoyl, or C₁-C₄ alkanoyl; and R¹ is C₁-C₄ alkyl, then R⁴ can not be —NH(substituted phenyl) or —N(C₁-C₄ alkyl) (substituted phenyl);
and wherein, when Y is CR²⁹:

J, K, L, M, Z, A, k, m, n, p, q, r, t, w, R³, R¹⁰, R¹¹, R¹², R¹³, R¹⁶, R¹⁸, R¹⁹, R²¹, R²³, R²⁴, R²⁵, and R²⁷ are as defined above and R^{25a}, in addition to being as defined above, can also be C₁-C₄ alkyl, but
V is N;
R¹ is C₁-C₂ alkyl, C₂-C₄ alkenyl, C₂-C₄ alkynyl, C₂-C₄ alkoxy, halogen, amino, methylamino, dimethylamino, aminomethyl, or N-methylaminomethyl;
R² is independently selected at each occurrence from the group consisting of hydrogen, halo, C₁-C₃, alkyl, nitro, amino, and —CO₂R¹⁰;
R₄ is taken together with R²⁹ to form a 5-membered ring and is —C(R²⁶)=or —N=when R²⁹ is —C(R³⁰)═ or —N═, or —CH(R²⁶)— when R²⁹ is —CH(R³⁰)—;
X is Cl, Br, I, S(O)ₙR⁸, OR⁸, halomethyl, —(CHR¹⁶)ₚOR⁸, cyano, —(CHR¹)ₚNR¹⁴R¹⁵, C(═O)R⁸, C₁-C₆ alkyl, C₂-C₁₀ alkenyl, C₂-C₁₀ alkynyl, C₁-C₁₀, alkoxy, aryl-(C₁-C₁₀)-alkyl, C₃-C₆ cycloalkyl, aryl-(C₁-C₁₀)-alkoxy, nitro, thio-(C₁-C₁₀)-alkyl, —C(═NOR¹⁶)—C₁-C₄-alkyl, —C(═NOR¹⁶)H, or C(═O)NR¹⁴R¹⁵ where substitution by R¹⁸ can occur on any carbon containing substituents;
X′ is hydrogen, Cl, Br, I, S(O)NR⁸, —(CHR¹⁶)ₚOR⁸, halomethyl, cyano, —(CHR¹⁶)ₚNR¹⁴R¹⁵, C(═O)R⁸, C₁-C₆ alkyl, C₂-C₁₀alkenyl, C₂-C₁₀, alkynyl, C₁-C₁₀ alkoxy, aryl-(C₁-C₁₀)-alkyl, C₃-C₆ cycloalkyl, aryl-(C₂-C₁₀)-alkoxy, nitro, thio-(C₂-C₁₀)-alkyl, —C(═NOR¹⁶)—C₁-C₄-alkyl, —C(═NOR¹⁶)H, or C(═O)NR⁸R¹⁵ where substitution by R¹⁸ can occur on any carbon containing substituents;
R⁵ is halo, —C(═NOR¹⁶)—C₁-C₄-alkyl, C₁-C₆ alkyl, C₁-C₃ haloalkyl, C₁-C₆ alkoxy, (CHR¹⁶)ₚOR⁵, (CHR¹⁶)ₚS(O)ₙR⁸, (CHR¹⁶)ₚNR¹⁴R¹⁵, C₃-C₆ cycloalkyl, C₂-C₁₀ alkenyl, C₂-C₁₀ alkynyl, aryl-(C₂-C₁₀)-alkyl, aryl-(C₁-C₁₀)-alkoxy, cyano, C₃-C₆ cycloalkoxy, nitro, amino-(C₁-C₁₀)-alkyl, thio-(C₁-C₁₀)-alkyl, SON(R⁸), C(═O)R⁸, —C(═NOR¹⁶)H, or C(═O)NR⁸R¹⁵ where substitution by R¹⁸ can occur on any carbon containing substituents;
R⁶ and R⁷ are independently selected at each occurrence from the group consisting of hydrogen, C₁-C₆ alkyl, C₃-C₁₀ cycloalkyl, —(CH₂)ₖR¹³, (C₄-C₁₂)-cycloalkylalkyl, C₁-C₆ alkoxy, —(C₁-C₆ alkyl)-aryl, heteroaryl, aryl, —S(O)_{z}-aryl or —(C₁-C₆ alkyl)-heteroaryl or aryl wherein the aryl or heteroaryl groups are optionally substituted with 1-3 groups selected from hydrogen, halogen, C₁-C₆ alkyl, C₁-C₆ alkoxy, amino, NHC(═O)(C₁-C₆ alkyl), NH(C₁-C₆ alkyl), N(C₁-C₆ alkyl)₂, nitro, carboxy, CO₂(C₁-C₆ alkyl), and cyano; or can be taken together to form —(CH₂)_{q}A(CH₂)ᵣ—, optionally substituted with 0-3 R¹⁷; or, when considered with the commonly attached nitrogen, can be taken together to form a heterocycle, said heterocycle being substituted on carbon with 1-3 groups consisting of hydrogen, C₁-C₆ alkyl, hydroxy, or C₁-C₆ alkoxy;
R⁸ is independently selected at each occurrence from the group consisting of hydrogen, C₁-C₆ alkyl, —(C₄-C₁₂)cycloalkylalkyl, (CH₂)ₜR²², C₃-C₁₀ cycloalkyl, —(C₁-C₆ alkyl)-aryl, heteroaryl, —NR¹⁶, —N(CH₂)ₙNR⁶R⁷; —(CH₂)ₖR²⁵, —(C₁-C₆ alkyl)-heteroaryl or aryl optionally substituted with 1-3 groups selected from hydrogen, halogen, C₁-C₆ alkyl, C₁-C₆ alkoxy, amino, NHC(═O)(C₁-C₆ alkyl), NH(C₁-C₆ alkyl), N(C₁-C₆alkyl)₂, nitro, carboxy, CO₂(C₁-C₆ alkyl), and cyano;
R⁹ is independently selected at each occurrence from R¹⁰, hydroxy, C₁-C₄ alkoxy, C₃-C₆ cycloalkyl, C₂-C₄ alkenyl, and aryl substituted with 0-3 R¹⁸;
R¹⁴ and R¹⁵ are independently selected at each occurrence from the group consisting of hydrogen, C₁-C₆ alkyl, C₃-C₆ cycloalkyl, (CH₂)ₜR²², and aryl substituted with 0-3 R¹8;
R¹⁷ is independently selected at each occurrence from the group consisting of R¹⁰, C₁-C₄ alkoxy, halo, OR²³, SR²³, and NR²³R²⁴;
R²⁰ is independently selected at each occurrence from the group consisting of R¹⁰ and C(═O)R³¹;
R²² is independently selected at each occurrence from the group consisting of cyano, OR²⁴, SR²⁴, NR²³R²⁴, C₃-C₈ cycloalkyl, —S(O)ₙR³¹, and —C(═O)R²⁵;
R²⁶ is hydrogen or halogen;
R²⁸ is C₁-C₂, alkyl, C₂-C₄ alkenyl, C₂-C₄ alkynyl, hydrogen, C₁-C₂ alkoxy, halogen, or C₂-C₄ alkylamino;
R²⁹ is taken together with R⁴ to form a five membered ring and is: —CH(R³⁰)— when R⁴ is —CH(R²⁸)—, —C(R³⁰)=or —N=when R⁴ is —C(R²⁸)=or —N═;
R³⁰ is hydrogen, cyano, C₁-C₂ alkyl, C₁-C₂ alkoxy, halogen, C₁-C₂ alkenyl, nitro, amido, carboxy, or amino;
R³¹ is C₁-C₄ alkyl, C₃-C₇ cycloalkyl, or aryl-(C₁-C₄)alkyl; provided that when J, K, and L are all CH, M is CR⁵, Z is CH, R³ is CH₃, R²⁸ is H, R⁵ is isopropyl, X is Br, X′ is H, and R¹ is CH₃, then R³⁰ can not be H, —CO₂H, or —CH₂NH₂; and further provided that when J, K and L are all CH; M is CR⁵; Z is N; and
(A) R²⁹ is —C(R³⁰)=; then one of R²⁸ or R³⁰ is hydrogen;
(B) R²⁹ is N; then R³ is not halo, NH₂, NO₂, CF₃, CO₂H, CO₂-alkyl, alkyl, acyl, alkoxy, OH, or —(CH₂)ₘOalkyl;
(C) R²⁹ is N; then R²⁸ is not methyl if X or X′ are bromo or methyl and R⁵ is nitro; or
(D) R²⁹ is N; and R¹ is CH₃; and R³ is amino; then R⁵ is not halogen or methyl.

Preferred compounds of this group include those wherein:
i) V is N, R¹ is methyl; and R³ is aryl, NR⁶R⁷, or OR⁸;
ii) V is N, R¹ is methyl; R³ is aryl, NR⁶R⁷, or OR⁸; and R⁴ is methyl or ethyl;
iii) V is N, R¹ is methyl; R³ is aryl, NR⁶R⁷, or OR⁸; R⁴ is methyl or ethyl; and X is O(C₁-C₄ alkyl), Br, or C₁-C₄ alkyl;
iv) V is N, R¹ is methyl; R³ is aryl, NR⁶R⁷, or OR⁸; R⁴ is methyl, ethyl; X is OMe, Br, or (C₁-C₄ alkyl), M is C₁-C₄ alkyl, Br, Cl, or O(C₁-C₄ alkyl); and
v) V is N, R¹ is methyl; R³ is aryl, NR⁶R⁷, OR⁸; or R⁴ is methyl, ethyl; X is OMe, Br, or C₁-C₄ alkyl, M is C₁-C₄ alkyl, Br, Cl, or O(C₁-C₄ alkyl); and L is CH, or N.

Specific CRF antagonists useful in the practice of the invention, include, without limitation, the following compounds:
4-(1-ethyl-propoxy)-3,6-dimethyl-2-(2,4,6-trimethylphenoxy)-pyridine;
(3,6-dimethyl-2-(2,4,6-trimethyl-phenoxy)-pyridin-4-yl)-(1-ethyl-propyl)-amine;
(3,6-dimethyl-2-(4-chloro-2,6-dimethyl-phenoxy)-pyridin-4-yl)-(1-ethyl-propyl)-amine;
5-(1-ethyl-propoxy)-7-methyl-1-(2,6-dimethyl-4-cholorophenyl)-1-4-dihydro-2H-3-oxa-1,8-diazanaphthalene;
butyl-[2,5-dimethyl-7-(2,4,6-trimethylphenyl)-6,7-dihydro-5H-pyrrolo[2,3-d]pyrimidin-4-yl]-ethyl-amino;
4-(butyl-ethylamino)-2,5-dimethyl-7-(2,4,6-trimethylphenyl)-5,7-dihydro-pyrrolo[2,3-d]pyrimidin-6-one;
4-(1-ethylpropoxy)-2,5-dimethyl-6-(2,4,6-trimethylphenoxy)-pyrimidine;
N-butyl-N-ethyl-2,5-dimethyl-N N-(2,4,6-trimethylphenyl)-pyrimidine-4,6-diamine;
[4-(1-ethyl-propoxy)-3,6-dimethyl-pyridin-2-yl]-(2,4,6-trimethylphenyl)-amine;
6-(ethyl-propyl-amino)-2,7-dimethyl-9-(2,4,6-trimethylphenyl)-7,9-dihydro-purin-8-one;
3-{(4-methyl-benzyl)-[3,6-dimethyl-1-(2,4,6-trimethylphenyl)-1H-pyrazolo[3,4-d]pyrimidin-4-yl]-amino}-propan-1-ol;
diethyl-[6-methyl-3-methysulfany-1-(2,4,6-trichlorophenyl)-1H-pyrazolo[3,4-d]pyrimidin-4-yl]-amine;
2-{butyl-[6-methyl-3-methylsulfanyl-1-(2,4,6-trichlorophenyl)-1H-pyrazolo[3,4-d]pyrimidin-4-yl]-amino}-ethanol;
dibutyl-[6-methyl-3-methylsulfanyl-1-(2,4,6-trichlorophenyl)-1H-pyrazolo[3,4-d]pyrimidin-4-yl]-amine;
butyl-ethyl-[6-methyl-3-methylsulfanyl-1-(2,4,6-trichlorophenyl)-1H-pyrazolo[3,4-d]pyrimidin[4-yl]-amine;
butyl-ethyl-[6-methyl-3-methylsulfonyl-1-(2,4,6-trichlorophenyl)-1H-pyrazolo[3,4-d]pyrimidin-4-yl]-amine;
butyl-cyclopropylmethyl-[6-methyl-3-methylsulfanyl-1-(2,4,6-trichlorophenyl)-1H-pyrazolo[3,4-d]pyrimidin-4-yl]-amine;
di-1-propyl-[6-methyl-3-methylsulfanyl-1-(2,4,6-trichlorophenyl)-1H-pyrazolo[3,4-d]pyrimidin-4-yl]-amine;
diallyl-[6-methyl-3-methylsulfanyl-1-(2,4,6-trichlorophenyl)-1H-pyrazolo[3,4-d]pyrimidin-4-yl]-amine;
butyl-ethyl-[6-chloro-3-methylsulfanyl-1-(2,4,6-trichlorophenyl)-1H-pyrazolo[3,4-d]pyrimidin-4-yl]-amine;
butyl-ethyl-[6-methoxy-3-methylsulfanyl-1-(2,4,6-trichlorophenyl)-1H-pyrazolo[3,4-d]pyrimidin-4-yl]-amine;
propyl-ethyl-[3,6-dimethyl-1-(2,4,6-trimethylphenyl)-1H-pyrazolo[3,4-d]pyrimidin-4-yl]-amine;
4-(1-ethyl-propyl)-6-methyl-3-methylsulfanyl-1-(2,4,6-trimethylphenyl)-1H-pyrazolo[3,4-d]pyrimidine;
n-butyl-ethyl-[2,5-dimethyl-7-(2,4,6-trimethyl phenyl)-7H-pyrrolo[2,3-d]pyrimidin-4-yl]amine;
di-n-propyl-[2,5-dimethyl-7-(2,4,6-trimethylphenyl)-7H-pyrrolo[2,3-d]pyrimidin-4-yl]amine;
ethyl-n-propyl-[2,5-dimethyl-7-(2,4,6-trimethylphenyl)-7H-pyrrolo[2,3-d]pyrimidin-4-yl]amine;
diethyl-2,5-dimethyl-7-(2,4,6-trimethylphenyl)-7H-pyrrolo[2,3-d]pyrimidin-4-yl]amine;
n-butyl-ethyl-[2,5,6-trim ethyl-7-(2,4,6-trimethyl phenyl)-7H-pyrrolo[2,3-d]pyrimidin-4-yl]amine;
2-{N-n-butyl-N-[2,5-dimethyl-7-(2,4,6-trimethylphenyl)-7H-pyrrolo[2,3-d]pyrimidin-4-yl]amino}-ethanol;
4-(1-ethyl-propyl)-2,5,6-trimethyl-7-(2,4,6-trimethylphenyl)-7H-pyrrolo[2,3-d]pyrimidine;
n-butyl-ethyl-[2,5-dimethyl-7-(2,4-dimethylphenyl)-7H-pyrrolo[2,3-d]pyrimidin-4-yl]amine;
2,5-dimethyl-7-(2,4,6-trimethylphenyl)-7H-pyrrolo[2,3-d]pyrimidyl-4-yl]-(1-ethyl-propyl)amine;
butyl-[3,6-dimethyl-1-(2,4,6-trimethylphenyl)-1H-pyrazolo[3,4-b]pyridin-4-yl]-ethylamine;
[3,6-dimethyl-1-(2,4,6-trimethylphenyl)-1H-pyrazolo[3,4,b]pyridin-4-yl]-(1-methoxymethylpropyl)-amine;
4-(1-methoxymethylpropoxy)-3,6-dimethyl-1-(2,4,6-trimethylphenyl)-1H-pyrazolo[3,4-b]pyridine;
(1-ethylpropyl)-[3,5,6-trimethyl-1-(2,4,6-trimethylphenyl)-1H-pyrazolo[3,4-b]pyridin-4-yl]-amine;
4-(1-ethylpropoxy)-2,5-dimethyl-7-(2,4,6-trimethylphenyl)-7H-pyrrolo[2,3-b]pyridine;
4-(1-ethylpropoxy)-2,5,6-trimethyl-7-(2,4,6-trimethylphenyl)-7H-pyrrolo[2,3-b]pyridine;
4-(1-ethylpropoxy)-2,5-dimethyl-7-(2,6-dimethyl-4-bromophenyl)-7H-pyrrolo[2,3-b]pyridine;
2,5,6-trimethyl-7-(1-propylbutyl)-4-(2,4,6-trimethylphenoxy)-7H-pyrrolo[2,3-d]pyrimidine;
1-(1-ethylpropyl)-6-methyl-4-(2,4,6-trimethylphenylamino)-1,3-dihydro-imidazo[4,5-c]pyridin-2-one;
9-(1-ethylpropyl)-2-methyl-6-(2,4,6-trimethylphenylamino)-7,9-dihydro-purin-8-one;
1-(1-ethyl propyl)-6-methyl-4-(2,4,6-trimethylphenoxy)-1,3-dihydro-imidazo[4,5-c]pyridin-2-one;
1-(1-ethylpropyl)-6-methyl-4-(2,4,6-trimethylphenoxy)-1H-imidazo[4,5-c]pyridine;
1-(1-ethylpropyl)-3,6-dimethyl-4-(2,4,6-trimethylphenoxy)-1,3-dihydro-imidazo[4,5-c]pyridin-2-one;
1-(1-ethylpropyl)-3,6-dimethyl-4-(2,4,6-trimethylphenylamino)-1,3-dihydro-imidazo[4,5-c]pyridin-2-one;
1-(1-ethyl-propyl)-4,7-dimethyl-5-(2,4,6-trimethyl-phenoxy)-1,4-dihydro-2H-pyrido[3,4-b]pyrazin-3-one;
1-(1-ethyl-propyl)-4,7-dimethyl-5-(2,4,6-trimethyl-phenoxy)-1,4-dihydro-2H-pyrido[3,4-b]pyrazin-3-one;
1-(1-ethyl-propyl)-4,7-dimethyl-5-(2,4,6-trimethyl-phenoxy)-1,2,3,4-tetrahydro-pyrido[3,4-b]pyrazine;
1-(1-ethyl-propyl)-7-methyl-5-(2,4,6-trimethyl-phenoxy)-1,2,3,4-tetrahydro-pyrido[3,4-b]pyrazine;
1-(1-ethyl-propyl)-7-methyl-2-oxo-5-(2,4,6-trimethyl-phenoxy)-1,2,3,4-tetrahydro-[1,6]naphthyridine-3-carboxylic acid methyl ester;
1-(1-ethyl-propyl)-7-methyl-2-oxo-5-(2,4,6-trimethyl-phenoxy)-1,2,3,4-tetrahydro-[1,6]naphthyridine-3-carboxylic acid isopropyl ester;
1-(1-ethyl-propyl)-7-methyl-5-(2,4,6-trimethyl-phenoxy)-3,4-dihydro-1H-[1,6]naphthyridin-2-one;
1-(1-ethyl-propyl)-7-methyl-5-(2,4,6-trimethyl-phenoxy)-1,2,3,4-tetrahydro-[1,6]naphthyridine;
1-(1-ethyl-propyl)-7-methyl-5-(2,4,6-trimethyl-phenoxy)-1,4-dihydro-2H-3-oxa-1,6-diaza-naphthalene;
1-(1-ethyl-propyl)-4,7-dimethyl-5-(2,4,6-trimethyl-phenoxy)-1,4-dihydro-2H-3-oxa-1,6-diaza-naphthalene;
1-(1-ethyl-propyl)-3,7-dimethyl-5-(2,4,6-trimethyl-phenoxy)-3,4-dihydro-1H-3-oxa-[1,6]-naphthyridin-2-one;
1-(1-ethyl-propyl)-3,3,6-trimethyl-4-(2,4,6-trimethyl-phenoxy)-2,3-dihydro-1H-pyrrolo[3,2-c]pyridine;
7-(1-ethyl-propoxy)-5-methyl-3-(2,4,6-trimethyl-phenyl)-pyrazolo[1,5-a]pyrimidine;
[2,5-dimethyl-3-(2,4,6-trimethyl-phenyl)-pyrazolo[1,5-a]pyrimidin-7-yl]-(1-ethyl-propyl)-amine;
(1-ethyl-propyl)-[5-methyl-3-(2,4,6-trimethyl-phenyl)-pyrazolo[1,5-a]pyrimidin-7-yl]-amine;
7-(1-ethyl-propoxy)-2,5-dimethyl-3-(2,4,6-trimethyl-phenyl)-pyrazolo[1,5-a]pyrimidine;
[2,5-dimethyl-3-(2,4,6-trimethyl-phenyl)-pyrazolo[1,5-a]pyrimidin-7-yl]-ethyl-propyl-amine;
[6-bromo-5-bromomethyl-3-(2,4,6-trimethyl-phenyl)-3H-[1,2,3]triazolo[4,5-b]pyridin-7-yl]-(1-ethyl-propyl)-amine;
(1-ethyl-propyl)-[5-methyl-3-(2,4,6-trimethyl-phenyl)-3H-[1,2,3]triazolo[4,5-b]pyridin-7-yl]-amine;
[6-bromo-5-methyl-3-(2,4,6-trimethyl-phenyl)-3H-[1,2,3]triazolo[4,5-b]pyridin-7-yl]-(1-ethyl-propyl)-methyl-amine;
7-(1-ethyl-propoxy)-5-methyl-3-(2,4,6-trimethyl-phenyl)-3H-[1,2,3]triazolo[4,5-b]pyridine;
4-(1-ethyl-propoxy)-2,5-dimethyl-7-(2,4,6-trimethyl-phenyl)-5H-pyrrolo[3,2-d]pyrimidine;
(±)-2,5-dimethyl-4-(tetrahydro-furan-3-yloxy)-7-(2,4,6-trimethyl-phenyl)-5H-pyrrolo-[3,2-d]pyrimidine;
2,5-dimethyl-4-(S)-(tetrahydro-furan-3-yloxy)-7-(2,4,6-trimethyl-phenyl)-5H-pyrrolo-[3,2-d]pyrimidine;
2,5-dimethyl-4-(1-propyl-butoxy)-7-(2,4,6-trimethyl-phenyl)-5H-pyrrolo[3,2-d]pyrimidine;
4-sec-butylsulfanyl-2,5-dimethyl-7-(2,4,6-trimethyl-phenyl)-5H-pyrrolo[3,2-d]pyrimidine;
4-(butyl-ethyl-amino)-2,6-dimethyl-8-(2,4,6-trimethyl-phenyl)-5,8-dihydro-6H-pyrido[2,3-d]pyrimidin-7-one;
8-(1-ethyl-propoxy)-6-methyl-4-(2,4,6-trimethyl-phenyl)-3,4-dihydro-1H-pyrido[2,3-b]pyrazin-2-one;
8-(1-ethyl-propoxy)-6-methyl-4-(2,4,6-trimethyl-phenyl)-1,2,3,4-tetrahydro-pyrido[2,3-b]pyrazine;
4-(1-ethyl-propoxy)-2-methyl-8-(2,4,6-trimethyl-phenyl)-quinoline;
5-(1-ethyl-propoxy)-7-methyl-1-(2,4,6-trimethyl-phenyl)-1,4-dihydro-2H-3-oxa-1,8-diaza-naphthalene;
5-(1-ethyl-propoxy)-7-methyl-1-(2,4,6-trimethyl-phenyl)-1,2-dihydro-3-oxa-1,8-diaza-naphthalen-4-one;
8-(1-ethyl-propoxy)-1,6-dimethyl-4-(2,4,6-trimethyl-phenyl)-1,2,3,4-tetrahydro-pyrido[2,3-b]pyrazine;
(1-ethyl-propyl)-[2-methyl-8-(2,4,6-trimethyl-phenyl)-quinolin-4-yl]-amine;
4-(butyl-ethyl-amino)-2,6-dimethyl-8-(2,6-dimethyl-4-bromo-phenyl)-5,8-dihydro-6H-pyrido[2,3-d]pyrimidin-7-one;
4-(butyl-ethyl-amino)-2-methyl-8-(2,6-dimethyl-4-bromo-phenyl)-5,8-dihydro-6H-pyrido[2,3-d]pyrimidin-7-one;
4-(1-ethyl-propoxy)-2-methyl-8-(2,6-dimethyl-4-bromo-phenyl)-5,8-dihydro-6H-pyrido[2,3-d]pyrimidin-7-one;
(butyl-ethyl)-[2-methyl-8-(2,6-dimethyl-4-bromo-phenyl)-5,6,7,8-tetrahydro-pyrido[2,3-d]pyrimidin-4-yl]-amine;
(propyl-ethyl)-[2-methyl-8-(2,6-dimethyl-4-bromo-phenyl)-5,6,7,8-tetrahydro-pyrido[2,3-d]pyrimidin-4-yl]-amine;
(diethyl)-[2-methyl-8-(2,6-dimethyl-4-bromo-phenyl)-5,6,7,8-tetrahydro-pyrido[2,3-d]pyrimidin-4-yl]-amine;
(1-ethyl-propyl)-[2-methyl-8-(2,6-dimethyl-4-bromo-phenyl)-5,6,7,8-tetrahydro-pyrido[2,3-d]pyrimidin-4-yl]-amine;
(1-ethyl-propoxy)-2-methyl-8-(2,6-dimethyl-4-bromo-phenyl)-5,6,7,8-tetrahydro-pyrido[2,3-d]pyrimidine;
4-(butyl-ethyl-amino)-2-methyl-8-(2,4,6-trimethyl-phenyl)-5,8-dihydro-6H-pyrido[2,3-d]pyrimidin-7-one;
4-(1-ethyl-propoxy)-2-methyl-8-(2,4,6-trimethyl-phenyl)-5,8-dihydro-6H-pyrido[2,3-d]pyrimidin-7-one;
(butyl-ethyl)-[2-methyl-8-(2,4,6-trimethyl-phenyl)-5,6,7,8-tetrahydro-pyrido[2,3-d]pyrimidin-4-yl]-amine;
(propyl-ethyl)-[2-methyl-8-(2,4,6-trimethyl-phenyl)-5,6,7,8-tetrahydro-pyrido[2,3-d]pyrimidin-4-yl]-amine;
(diethyl)-[2-methyl-8-(2,4,6-trimethyl-phenyl)-5,6,7,8-tetrahydro-pyrido[2,3-d]pyrimidin-4-yl]-amine;
(1-ethyl-propyl)-[2-methyl-8-(2,4,6-trimethyl-phenyl)-5,6,7,8-tetrahydro-pyrido[2,3-d]pyrimidin-4-yl]-amine;
(1-ethyl-propoxy)-2-methyl-8-(2,4,6-trimethyl-phenyl)-5,6,7,8-tetrahydro-pyrido[2,3-d]pyrimidine;
8-(1-ethyl-propoxy)-6-methyl-4-(2,6-dimethyl-4-bromo-phenyl)-3,4-dihydro-1H-pyrido[2,3-b]pyrazin-2-one;
8-(1-ethyl-propoxy)-6-methyl-4-(2,6-dimethyl-4-bromo-phenyl)-1,2,3,4-tetrahydro-pyrido[2,3-b]pyrazine;
4-(1-ethyl-propoxy)-2-methyl-8-(2,6-dimethyl-4-bromo-phenyl)-quinoline;
5-(1-ethyl-propoxy)-7-methyl-1-(2,6-dimethyl-4-bromo-phenyl)-1,4-dihydro-2H-3-oxa-1,8-diaza-naphthalene;
5-(1-ethyl-propoxy)-7-methyl-1-(2,6-dimethyl-4-bromo-phenyl)-1,2-dihydro-3-oxa-1,8-diaza-naphthalen-4-one;
8-(1-ethyl-propoxy)-1,6-dimethyl-4-(2,6-dimethyl-4-bromo-phenyl)-1,2,3,4-tetrahydro-pyrido[2,3-b]pyrazine;
(1-ethyl-propyl)-[2-methyl-8-(2,6-dimethyl-4-bromo-phenyl)-quinolin-4-yl]-amine;
4-(butyl-ethyl-amino)-2,6-dimethyl-8-(2,6-dimethyl-4-chloro-phenyl)-5,8-dihydro-6H-pyrido[2,3-d]pyrimidin-7-one;
8-(1-ethyl-propoxy)-6-methyl-4-(2,6-dimethyl-4-chloro-phenyl)-3,4-dihydro-1H-pyrido[2,3-b]pyrazin-2-one;
8-(1-ethyl-propoxy)-6-methyl-4-(2,6-dimethyl-4-chloro-phenyl)-1,2,3,4-tetrahydro-pyrido[2,3-b]pyrazine;
4-(1-ethyl-propoxy)-2-methyl-8-(2,6-dimethyl-4-chloro-phenyl)-quinoline;
5-(1-ethyl-propoxy)-7-methyl-1-(2,6-dimethyl-4-chloro-phenyl)-1,4-dihydro-2H-3-oxa-1,8-diaza-naphthalene;
5-(1-ethyl-propoxy)-7-methyl-1-(2,6-dimethyl-4-chloro-phenyl)-1,2-dihydro-3-oxa-1,8-diaza-naphthalen-4-one;
8-(1-ethyl-propoxy)-1,6-dimethyl-4-(2,6-dimethyl-4-chloro-phenyl)-1,2,3,4-tetrahydro-pyrido[2,3-b]pyrazine;
(1-ethyl-propyl)-[2-methyl-8-(2,6-dimethyl-4-chloro-phenyl)-quinolin-4-yl]-amine;
8-(1-hydroxymethyl-propoxy)-6-methyl-4-(2,4,6-trimethyl-phenyl)-3,4-dihydro-1H-pyrido[2,3-b]pyrazin-2-one;
8-(1-hydroxymethyl-propylamino)-6-methyl-4-(2,4,6-trimethyl-phenyl)-3,4-dihydro-1H-pyrido[2,3-b]pyrazin-2-one;
8-(1-ethyl-propylamino)-6-methyl-4-(2,4,6-trimethyl-phenyl)-3,4-dihydro-1H-pyrido[2,3-b]pyrazin-2-one;
8-diethylamino-6-methyl-4-(2,4,6-trimethyl-phenyl)-3,4-dihydro-1H-pyrido[2,3-b]pyrazin-2-one;
8-(ethyl-propyl-amino)-6-methyl-4-(2,4,6-trimethyl-phenyl)-3,4-dihydro-1H-pyrido[2,3-b]pyrazin-2-one;
8-(butyl-ethyl-amino)-6-methyl-4-(2,4,6-trimethyl-phenyl)-3,4-dihydro-1H-pyrido[2,3-b]pyrazin-2-one;
8-(1-hydroxymethyl-propoxy)-6-methyl-4-(2,4,6-trimethyl-phenyl)-1,2,3,4-tetrahydro-pyrido[2,3-b]pyrazine;
8-(1-hydroxymethyl-propylamino)-6-methyl-4-(2,4,6-trimethyl-phenyl)-1,2,3,4-tetrahydro-pyrido[2,3-b]pyrazine;
8-(1-ethyl-propylamino)-6-methyl-4-(2,4,6-trimethyl-phenyl)-1,2,3,4-tetrahydro-pyrido[2,3-b]pyrazine;
8-diethylamino-6-methyl-4-(2,4,6-trimethyl-phenyl)-1,2,3,4-tetrahydro-pyrido[2,3-b]pyrazine;
8-(ethyl-propyl-amino)-6-methyl-4-(2,4,6-trimethyl-phenyl)-1,2,3,4-tetrahydro-pyrido[2,3-b]pyrazine;
8-(butyl-ethyl-amino)-6-methyl-4-(2,4,6-trimethyl-phenyl)-1,2,3,4-tetrahydro-pyrido[2,3-b]pyrazine;
4-(1-hydroxymethyl-propoxy)-2-methyl-8-(2,4,6-trimethyl-phenyl)-quinoline;
4-(1-hydroxymethyl-propylamino)-2-methyl-8-(2,4,6-trimethyl-phenyl)-quinoline;
4-(1-ethyl-propylamino)-2-methyl-8-(2,4,6-trimethyl-phenyl)-quinoline;
4-diethylamino-2-methyl-8-(2,4,6-trimethyl-phenyl)-quinoline;
4-(ethyl-propyl-amino)-2-methyl-8-(2,4,6-trimethyl-phenyl)-quinoline;
4-(butyl-ethyl-amino)-2-methyl-8-(2,4,6-trimethyl-phenyl)-quinoline;
5-(1-hydroxymethyl-propoxy)-7-methyl-1-(2,4,6-trimethyl-phenyl)-1,4-dihydro-2H-3-oxa-1,8-diaza-naphthalene;
5-(1-hydroxymethyl-propylamino)-7-methyl-1-(2,4,6-trimethyl-phenyl)-1,4-dihydro-2H-3-oxa-1,8-diaza-naphthalene;
5-(1-ethyl-propylamino)-7-methyl-1-(2,4,6-trimethyl-phenyl)-1,4-dihydro-2H-3-oxa-1,8-diaza-naphthalene;
5-diethylamino-5-methyl-1-(2,4,6-trimethyl-phenyl)-1,4-dihydro-2H-3-oxa-1,8-diaza-naphthalene;
5-(ethyl-propyl-amino)-7-methyl-1-(2,4,6-trimethyl-phenyl)-1,4-dihydro-2H-3-oxa-1,8-diaza-naphthalene;
8-(butyl-ethyl-amino)-6-methyl-4-(2,4,6-trimethyl-phenyl)-1,4-dihydro-2H-3-oxa-1,8-diaza-naphthalene;
4-(2,4-dichlorophenyl)-5-methyl-2-[N-(1-(methoxymethyl)-1-(naphth-2-yl)methyl)-N-propylamino]thiazole;
oxalate of 4-(2,4-dichlorophenyl)-5-methyl-2-[N-(6-methoxyisoquinol-5-yl)-N-propylamino]thiazole;
oxalate of 4-(2-chloro-4-methoxyphenyl)-5-methyl-2-[N-(6-methoxylisoquinol-5-yl)-N-propylamino]thiazole;
4-(2-chloro-4-methoxyphenyl)-5-methyl-2-[N-(1-methoxycarbonylmethyl indol-5-yl)-N-propylamino]thiazole;
oxalate of 4-(2-chloro-4-methoxyphenyl)-5-methyl-2-[N-(6-methoxy isoquinol-5-yl)-N-propylamino]thiazole;
oxalate of 4-(2-chloro-4-methoxyphenyl)-5-methyl-2-[N-(6-chloroisoquinol-5-yl)-N-propylamino]thiazole;
oxalate of 4-(2-chloro-4-methoxyphenyl)-5-methyl-2-[N-1-methoxynaphth-2-yl)-N-propylamino]thiazole;
4-(2-chloro-4-methoxyphenyl)-5-methyl-2-[N-1-methoxynaphth-2-yl)-N-propylamino]thiazole;
oxalate of 4-(2-chloro-4-trifluoromethylphenyl)-5-methyl-2-[N-6-methoxyisoquinol-5-yl)-N-propylamino]thiazole;
chlorhydrate of 4-(2-chloro-4-methoxyphenyl)-5-methyl-2[N-(2-ethoxynaphth-1-yl)-N-propylamino]thiazole;
chlorhydrate of 4-(2-chloro-4-methoxyphenyl)-5-methyl-2[N-(2,3-dimethylnaphth-1-yl)-N-propylamino]thiazole;
chlorhydrate of 4-(2-chloro-4-methoxyphenyl)-5-methyl-2-[N-(6-bromo-2-methoxynaphth-1-yl)-N-propylamino]thiazole;
chlorhydrate of 4-(2-chloro-4-methoxyphenyl)-5-methyl-2-[N-(2,6-dimethylnaphth-1-yl)-N-propylamino]thiazole;
chlorhydrate of 4-(2-chloro-4-methoxyphenyl)-5-methyl-2-[N-(1-(methoxymethyl)-1-(naphth-2-yl)methyl)-N-propylamino]thiazole;
chlorhydrate of 4-(2-chloro-4-methoxyphenyl)-5-methyl-2-[N-(1-(cyclopropyl)-1-(naphth-2-yl)methyl)-N-propylamino]thiazole;
3-(2,4-dichlorophenyl)-5-methyl-7(N-propyl-N-cyclopropanemethylamino)-pyrazolo[2,3-a]pyrimidine;
3-(2,4-dichlorophenyl)-5-methyl-7-(N-allyl-N-cyclopropanemethylamino)-pyrazolo[2,3-a]pyrimidine;
2-methylthio-3-(2,4-dichlorophenyl)-5-methyl-7-(N,N-diallylamino)-pyrazolo[2,3-a]pyrimidine;
2-methylthio-3-(2,4-dichlorophenyl)-5-methyl-7-(N-butyl-N-cyclopropanemethyl-amino)pyrazolo[2,3-a]pyrimidine;
2_methylthio-3-(2,4-dichlorophenyl)-5-methyl-7-(N-propyl-N-cyclopopanemethylamino)pyrazolo[2,3-a]pyrimidine;
2-methyl-3-(4-chlorophenyl)-5-methyl-7-(N,N-dipropylamino)-pyrazolo[2,3-a) pyrimidine;
3-[6-(dimethylamino)-3-pyridinyl]-2,5-dimethyl-N,N-dipropylpyrazolo[2,3-a]pyrimidin-7-amine;
3-[6-(dimethylamino)-4-methyl-3-pyridinyl]-2,5-dimethyl-N,N-dipropyl-pyrazolo[2,3-a]pyrimidine-7-amine;
3-(2,4-dimethoxyphenyl)-2,5-dimethyl-7-(N-propyl-N-methyloxyethylamino)-pyrazolo(2,3-a)pyrimidine;
7-(N-diethylamino)-2,5-dimethyl-3-(2-methyl-4-methoxyphenyl-[1,5-a]-pyrazolopyrimidine;
7-(N-(3-cyanopropyl)-N-propylamino-2,5,dimethyl-3-(2,4-dimethylphenyl)-[1,5-a]-pyrazolopyrimidine;
[3,6-dimethyl-2-(2,4,6-trimethyl-phenoxy)-pyridin-4-yl]-(1-ethyl-propyl)-amine;
[2-(4-chloro-2,6-dimethyl-phenoxy)-3,6-dimethyl-pyridin-4-yl]-(1-ethyl-propyl)-amine;
cyclopropylmethyl-[3-(2,4-dimethyl-phenyl)-2,5-dimethyl-pyrazolo[1,5-a]pyrimidin-7-yl]-propyl-amine;
cyclopropylmethyl-[3-(2-methyl-4-chloro-phenyl)-2,5-dimethyl-pyrazolo[1,5-a]pyrimidin-7-yl]-propyl-amine;
cyclopropylmethyl-[3-(2,4-di-chloro-phenyl)-2,5-dimethyl-pyrazolo[1,5-a]pyrimidin-7-yl]-propyl-amine;
[3-(2-methyl-4-chloro-phenyl)-2,5-dimethyl-pyrazolo[1,5-a]pyrimidin-7-yl]-di-propyl-amine;
[2,5-dimethyl-3-(2,4-dimethyl-phenyl)-pyrazolo[1,5-a]pyrimidin-7-yl]-(1-ethyl-propyl)-amine;
[2,5-dimethyl-3-(2,4-dichloro-phenyl)-pyrazolo[1,5-a]pyrimidin-7-yl]-(1-ethyl-propyl)-amine; and
4-(1-Ethyl-propylamino)-6-methyl-2-(2,4,6-trimethyl-phenoxy)-nicotinic acid methyl ester.

Methods for making the CRF antagonists described above are disclosed, for example, in WO 95/33750 incorporated by reference herein.

Particularly preferred are those compositions, methods, and kits that contain one of the following CRF antagonists.
4-(1-ethyl-propoxy)-3,6-dimethyl-2-(2,4,6-trimethylphenoxy)-pyridine;
(3,6-dimethyl-2-(2,4,6-trimethyl-phenoxy)-pyridin-4-yl)-(1-ethyl-propyl)-amine:
(3,6-dimethyl-2-(4-chloro-2,6-dimethyl-phenoxy)-pyridin-4-yl)-(1-ethyl-propyl)-amine:
5-(1-ethyl-propoxy)-7-methyl-1-(2,6-dimethyl-4-cholorophenyl)-1-4-dihydro-2H-3-oxa-1,8-diazanaphthalene;
butyl-[2,5-dimethyl-7-(2,4,6-trimethylphenyl)-6,7-dihydro-5H-pyrrolo[2,3-d]pyrimidin-4-yl]-ethyl-amino;
4-(butyl-ethylamino)-2,5-dimethyl-7-(2,4,6-trimethylphenyl)-5,7-dihydro-pyrrolo[2,3-d]pyrimidin-6-one;
4-(1-ethylpropoxy)-2,5-dimethyl-6-(2,4,6-trimethylphenoxy)-pyrimidine;
N-butyl-N-ethyl-2,5-dimethyl-NN-(2,4,6-trimethylphenyl)-pyrimidine-4,6-diamine;
[4-(1-ethyl-propoxy)-3,6-dimethyl-pyridin-2-y]-(2,4,6-trimethylphenyl)-amine;
6-(ethyl-propyl-amino)-2,7-dimethyl-9-(2,4,6-trimethylphenyl)-7,9-dihydro-purin-8-one;
3-{(4-methyl-benzyl)-[3,6-dimethyl-1-(2,4,6-trimethyphenyl)-1H-pyrazolo[3,4-d]pyrimidin-4-yl]-amino}-propan-1-ol;
diethyl-[6-methyl-3-methylsulfanyl-1-(2,4,6-trichlorophenyl)-1H-pyrazolo[3,4-d]pyrimidin-4-yl]-amine; or
2-{butyl-[6-methyl-3-methylsulfanyl-1-(2,4,6-trichlorophenyl)-1H-pyrazolo[3,4-d]pyrimidin-4-yl]-amino}-ethanol,

Particularly preferred are those compositions, methods, and kits that contain one of the following 5-HT_{1B} receptor antagonists:
4-benzyl-2-[2-(4-methylpiperazin-1-yl)-benzylidene]-thiomorpholin-3-one;
4-(3,4-dichlorobenzyl)-2-[2-(4-methylpiperazin-1-yl)-benzylidene]-thiomorpholin-3-one;
2-[2-(4-methylpiperazin-1-yl)-benzylidene]-4-(4-trifluoromethylphenyl)-thiomorpholin-3-one;
2-[2-(4-methylpiperazin-1-yl)-benzylidene]-thiomorpholin-3-one;
4-(3,4-dichlorophenyl)-2-[2-fluoro-6-(4-methylpiperazin-1-yl)-benzylidene]-thiomorpholin-3-one;
4-(3,4-dichlorophenyl)-2-[2-(4-methylpiperazin-1-yl)-benzylidene]-thiomorpholin-3-one;
or pharmaceutically acceptable salts thereof.

In the preferred kits of the present invention, the pharmaceutical composition comprising a CRF antagonist is a pharmaceutical composition comprising one of the particularly preferred CRF antagonists as defined above, and the pharmaceutical composition comprising a 5-HT_{1B} receptor antagonist is a pharmaceutical composition comprising one of the particularly preferred 5-HT_{1B} receptor antagonists as defined above.

The preferred methods of treatment of the present invention are those methods that employ a particularly preferred CRF antagonist and particularly preferred 5-HT_{1B} receptor antagonist as defined above.

Also preferred are those methods that employ a particularly preferred CRF antagonist and a particularly preferred 5-HT_{1B} receptor antagonist or a pharmaceutical composition(s) of the present invention, as defined above, for treating migraine, depression, obsessive compulsive disorder, post-traumatic stress disorder (PTSD), and eating disorders.

Preferably, the combinations of pharmaceutically active compounds of the present invention show a synergistic effect and/or show less side effects, as compared to the individual compounds, when treating a mammal, preferably a human. Thus, in treating a particular disease, at a specific dosage level, the combinations of pharmaceutically active compounds of the present invention show a better activity than the activity which could be expected when administering the individual compounds, less or less severe side effects than could be expected when administering the individual compounds, or a combination of a better activity and of less or less severe side effects than could be expected when administering the individual compounds.

The expression “pharmaceutically acceptable salts” includes both pharmaceutically acceptable acid addition salts and pharmaceutically acceptable cationic salts.

### DETAILED DESCRIPTION OF THE INVENTION

Compounds of the formula I and II and their pharmaceutically acceptable salts, and CRF antagonists and their pharmaceutically acceptable salts are hereinafter also referred to, collectively, as “the active compounds.” Compounds of the formula I or II are useful in the treatment of hypertension, depression, generalized anxiety disorder, phobias such as agoraphobia, social phobia and simple phobias, posttraumatic stress syndrome, avoidant personality disorder, sexual dysfunction such as premature ejaculation, eating disorders such as anorexia nervosa and bulimia nervosa, obesity, chemical dependencies such as addictions to alcohol, cocaine, heroin, phenolbarbitol, nicotine and benzodiazepines, cluster headache, migraine, pain, Alzheimer's disease, obsessive-compulsive disorder, panic disorder, memory disorders such as dementia, amnestic disorders, and age-related cognitive decline (ARCD), Parkinson's diseases such as dementia in Parkinson's disease, neuroleptic-induced parkinsonism and tardive dyskinesias, endocrine disorders such as hyperprolactinaemia, vasospasm, particularly in the cerebral vasculature, cerebellar ataxia, gastrointestinal tract disorders, such as involving changes in motility and secretion, negative symptoms of schizophrenia, premenstrual syndrome, Fibromyalgia Syndrome, stress incontinence, Tourette syndrome, trichotillomania, kleptomania, male impotence, cancer such as small cell lung carcinoma, chronic paroxysmal hemicrania and headache, such as headache associated with vascular disorders. Similarly, the compositions of the present invention are useful in the treatment of the disorders or conditions listed in this paragraph.

The affinities of the compounds of the formula I for the various serotonin-1 receptors can be determined using standard radioligand binding assays as described in the literature. The 5-HT_{1A} affinity can be measured using the procedure of Hoyer et al. (Brain Res., 376, 85 (1986)). The 5-HT_{1B} affinity can be measured using the procedure of Heuring and Peroutka (J. Neurosci., 7, 894 (1987)). The activity of the compounds of the formula I or II at the 5-HT_{1B} binding site, the activity for 5-HT_{1A} binding ability, and the agonist and antagonist activities of the compounds of the formula I or II at 5-HT_{1A} and 5-HT_{1B} receptors may be determined as described in U.S. Pat. No. 6,380,186. All 5-HT_{1B} receptor antagonists that were tested exhibited IC₅₀'s less than 0.60 μM for 5-HT_{1B} affinity and IC₅₀'s less than 1.0 μM for 5-HT_{1A} affinity. Similarly, the activity at the 5-HT_{1B} binding site, the activity for 5-HT_{1A} binding ability, and the agonist and antagonist activities of the compositions of the present invention may be determined using the procedures described for the compounds in formula I in U.S. Pat. No. 6,380,186.

In the present invention, the 5-HT_{1B} receptor antagonists of formula I or II and the CRF antagonists may also be further combined with one or more other therapeutic agents, for instance, different antidepressant agents such as tricyclic antidepressants such as amitriptyline, dothiepin, doxepin, trimipramine, butripyline, clomipramine, desipramine, imipramine, iprindole, lofepramine, nortriptyline or protriptyline, monoamine oxidase inhibitors such as isocarboxazid, phenelzine or tranylcyclopramine or monoamine reuptake inhibitors such as fluvoxamine, sertraline, fluoxetine or paroxetine, and/or with antiparkinsonian agents such as dopaminergic antiparkinsonian agents such as levodopa, preferably in combination with a peripheral decarboxylase inhibitor such as benserazide or carbidopa, and/or therapeutic agents which do not appreciably block monoamine uptake or affect monoamine oxidase such as mirtazapine, mianserin, bupropion, lithium salts, antiepileptic drugs such as caramazepine, valproate, lamotrigine, topiramate, gabapentin, pregabalin. It is to be understood that the present invention covers the combination of a 5-HT_{1B} receptor antagonists of formula I or II or a pharmaceutically acceptable salt thereof with a corticotropin releasing factor antagonist or a pharmaceutically acceptable salt thereof and with one or more such therapeutic agents.

The combination of the compounds of the formula I or II or the pharmaceutically acceptable salts thereof and a corticotropin releasing factor antagonist or a pharmaceutically acceptable salt thereof is also referred herein to as “the active combination.” The active combinations are useful psychotherapeutics and may be used in the treatment of disorders the treatment of which is facilitated by enhanced serotonergic neurotransmission such as hypertension, depression, generalized anxiety disorder, phobias such as agoraphobia, social phobia and simple phobias, posttraumatic stress syndrome, avoidant personality disorder, sexual dysfunction such as premature ejaculation, eating disorders such as anorexia nervosa and bulimia nervosa, obesity, chemical dependencies such as addictions to alcohol, cocaine, heroin, phenolbarbitol, nicotine and benzodiazepines, cluster headache, migraine, pain, Alzheimer's disease, obsessive-compulsive disorder, panic disorder, memory disorders such as dementia, amnestic disorders, and age-related cognitive decline (ARCD), Parkinson's diseases such as dementia in Parkinson's disease, neuroleptic-induced parkinsonism and tardive dyskinesias, endocrine disorders such as hyperprolactinaemia, vasospasm, particularly in the cerebral vasculature, cerebellar ataxia, gastrointestinal tract disorders, such as involving changes in motility and secretion, negative symptoms of schizophrenia, premenstrual syndrome, Fibromyalgia Syndrome, stress incontinence, Tourette syndrome, trichotillomania, kleptomania, male impotence, cancer such as small cell lung carcinoma, chronic paroxysmal hemicrania and headache, such as headache associated with vascular disorders.

Activity of the active combinations as antidepressants and related pharmacological properties can be determined by methods (1)-(3) below, which are described in Koe, B. et al. Journal of Pharmacology and Experimental Therapeutics, 226 (3), 686-700 (1983). Specifically, activity can be determined by studying (1) their ability to affect the efforts of mice to escape from a swim-tank (Porsolt mouse “behavior despair” test), (2) their ability to potentiate 5-hydroxytryptophan-induced behavioral symptoms in mice in vivo, and (3) their ability to block the uptake of serotonin, norepinephrine and/or dopamine by synaptosomal rat brain cells in vitro. The ability of the active combinations to counteract reserpine hypothermia in mice in vivo can be determined according to the methods described in U.S. Pat. No. 4,029,731. The activity of the active combinations as antidepressants and related pharmacological properties also can be determined by methods (4)-(8)) below. Specifically, activity can be determined by studying (4) their ability to reverse the stress-induced decrease in sucrose intake in rodents described in Papp, M. et al., European Journal of Pharmacology, 261, 141-147 (1994), (5) learned helplessness paradigm described in Martin P et al., Life Sciences, 48, 2505-2511 (1991), (6) reversing the behavioral deficits of olfactory bulbectomized rats described in Broekkamp C L et al., Pharmacology, Biochemistry and Behavior, 13, 643-646 (1980), (7) increasing down-regulation or desensitization of beta-adrenergic receptors described in Mishra R. et al., Neuropharmacology, 19, 983-987 (1980), and (8) increasing extracellular levels of serotonin, norepinephrine, and/or dopamine in the prefrontal cortex of freely-moving rodents by in vivo dialysis described in Millan M J et al., European Journal of Neuroscience, 12, 1079-1095 (2000).

Methods that may be used to determine CRF antagonist activity of the compounds employed to practice the present invention are as described in, e.g., Wynn et al., Endocrinology, 116:1653-59 (1985), and Grigoriadis et al., Peptides, 10:179-88 (1989). Methods that can be used to determine the CRF binding protein inhibiting activity of compounds employed to practice the present invention are described in Smith et al., Brain Research, 745(1,2):248-56 (1997). These methods determine the binding affinity of a test compound for a CRF receptor, which is highly related to its expected activity as a CRF antagonist.

The compositions of the present invention may be formulated in a conventional manner using one or more pharmaceutically acceptable carriers. Thus, the active compounds or the active combinations of the invention may be formulated for oral, buccal, intranasal, parenteral (e.g., intravenous, intramuscular, intraperitoneal, or subcutaneous or through an implant) nasal, vaginal, sublingual, rectal o topical administration or in a form suitable for administration by inhalation or insufflation.

For oral administration, the pharmaceutical compositions may take the form of, for example, tablets or capsules prepared by conventional means with pharmaceutically acceptable excipients such as binding agents such as pregelatinized maize starch, polyvinylpyrrolidone or hydroxypropyl methylcellulose; fillers such as lactose, microcrystalline cellulose or calcium phosphate; lubricants such as magnesium stearate, talc or silica; disintegrants such as potato starch or sodium starch glycolate; or wetting agents such as sodium lauryl sulphate. The tablets may be coated by methods well known in the art. Liquid preparations for oral administration may take the form of, for example, solutions, syrups or suspensions, or they may be presented as a dry product for constitution with water or other suitable vehicle before use. Such liquid preparations may be prepared by conventional means with pharmaceutically acceptable additives such as suspending agents such as sorbitol syrup, methyl cellulose or hydrogenated edible fats; emulsifying agents such as lecithin or acacia, non-aqueous vehicles such as almond oil, oily esters or ethyl alcohol; and preservatives such as methyl or propyl p-hydroxybenzoates or sorbic acid.

For buccal administration, the composition may take the form of tablets or lozenges formulated in conventional manner.

The active compounds or the active combinations of the invention may be formulated for parenteral administration by injection, including using conventional catheterization techniques or infusion. Formulations for injection may be presented in unit dosage form, for example, in ampoules or in multi-dose containers, with an added preservative. The compositions containing the active combinations may take such forms as suspensions, solutions or emulsions in oily or aqueous vehicles, and may contain formulating agents such as suspending, stabilizing and/or dispersing agents. Alternatively, the active ingredient may be in powder form for reconstitution with a suitable vehicle, for example, sterile pyrogen-free water, before use.

The active compounds or the active combinations of the invention may also be formulated in rectal compositions such as suppositories or retention enemas, for example, containing conventional suppository bases such as cocoa butter or other glycerides. Compositions for vaginal administration are preferably suppositories that may contain, in addition to the active substance, excipients such as cocoa butter or a suppository wax. Compositions for nasal or sublingual administration are also prepared with standard excipients well known in the art.

For intranasal administration or administration by inhalation, the active compounds or the active combinations of the invention are conveniently delivered in the form of a solution or suspension from a pump spray container that is squeezed or pumped by the patient or as an aerosol spray presentation from a pressurized container or a nebulizer, with the use of a suitable propellant, for example, dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount. The pressurized container or nebulizer may contain a solution or suspension of the active compounds or the active combinations. Capsules and cartridges, made, for example, from gelatin, for use in an inhaler or insufflator may be formulated containing a powder mix of an active compound and a suitable powder base such as lactose or starch.

The pharmaceutical compositions of the present invention can consist of a combination of immediate release and controlled release characteristics. Such compositions can take the form of combinations of the active ingredients that range in size from nanoparticles to microparticles or in the form of a plurality of pellets with different release rates. The tablet or capsule composition of the present invention can contain a 5-HT_{1B} receptor antagonist of the formula I or II in sustained or controlled release form and the CRF antagonist in an immediate release form. Alternatively, the 5-HT_{1B} receptor antagonist can be in immediate release form and the CRF antagonist can be in sustained or controlled release form.

An exemplary dose of the active combinations of the invention for oral, parenteral or buccal administration to the average adult human for the treatment of the conditions referred to above, such as depression, is about 0.1 to about 200 mg of the active compound of formula I or II and of about 0.1 to about 500 mg of the corticotropin releasing factor antagonist per unit dose which could be administered, for example, 1 to 4 times per day.

The composition of this invention may contain, for example, 4-(1-ethyl-propoxy)-3,6-dimethyl-2-(2,4,6-trimethylphenoxy)-pyridine; (3,6-dimethyl-2-(2,4,6-trimethyl-phenoxy)-pyridin-4-yl)-(1-ethyl-propyl)-amine; (3,6-dimethyl-2-(4-chloro-2,6-dimethyl-phenoxy)-pyridin-4-yl)-(1-ethyl-propyl)-amine; 5-(1-ethyl-propoxy)-7-methyl-1-(2,6-dimethyl-4-cholorophenyl)-1-4-dihydro-2H-3-oxa-1,8-diazanaphthalene; butyl-[2,5-dimethyl-7-(2,4,6-trimethylphenyl)-6,7-dihydro-5H-pyrrolo[2,3-d]pyrimidin-4-yl]-ethyl-amino; 4-(butyl-ethylamino)-2,5-dimethyl-7-(2,4,6-trimethylphenyl)-5,7-dihydro-pyrrolo[2,3-d]pyrimidin-6-one; 4-(1-ethylpropoxy)-2,5-dimethyl-6-(2,4,6-trimethylphenoxy)-pyrimidine; N-butyl-N-ethyl-2,5-dimethyl-N N-(2,4,6-trimethylphenyl)-pyrimidine-4,6-diamine; [4-(1-ethyl-propoxy)-3,6-dimethyl-pyridin-2-yl]-(2,4,6-trimethylphenyl)-amine; 6-(ethyl-propyl-amino)-2,7-dimethyl-9-(2,4,6-trimethylphenyl)-7,9-dihydro-purin-8-one; 3-{(4-methyl-benzyl)-[3,6-dimethyl-1-(2,4,6-trimethylphenyl)-1H-pyrazolo[3,4-d]pyrimidin-4-yl]-amino}-propan-1-ol; diethyl-[6-methyl-3-methylsulfanyl-1-(2,4,6-trichlorophenyl)-1H-pyrazolo[3,4-d]pyrimidin-4-yl]-amine; or 2-{butyl-[6-methyl-3-methylsulfanyl-1-(2,4,6-trichlorophenyl)-1H-pyrazolo[3,4-d]pyrimidin-4-yl]-amino}-ethanol, or a pharmaceutically acceptable salt thereof as the corticotropin releasing factor antagonist and 4-benzyl-2-[2-(4-methylpiperazin-1-yl)-benzylidene]-thiomorpholin-3-one, 4-(3,4-dichlorobenzyl)-2-[2-(4-methylpiperazin-1-yl)-benzylidene]-thiomorpholin-3-one, or 2-[2-(4-methylpiperazin-1-yl)-benzylidene]-4-(4-trifluoromethyl-phenyl)-thiomorpholin-3-one as the 5-HT_{1B} antagonist. An exemplary daily dose of the corticotropin releasing factor antagonist in a pharmaceutical composition of this invention for oral, parenteral, rectal or buccal administration to the average adult human for the treatment of the conditions referred to above is from about 0.1 to about 300 mg of corticotropin releasing factor antagonist per unit dose administered 1 to 3 times per day. Exemplary and preferred doses for corticotropin releasing factor antagonists are determined on a compound by compound basis. 4-benzyl-2-[2-(4-methylpiperazin-1-yl)-benzylidene]-thiomorpholin-3-one, 4-(3,4-dichlorobenzyl)-2-[2-(4-methylpiperazin-1-yl)-benzylidene]-thiomorpholin-3-one, or 2-[2-(4-methylpiperazin-1-yl)-benzylidene]-4-(4-trifluoromethyl-phenyl)-thiomorpholin-3-one may each be present in an amount between about 0.1 to about 200 mg, preferably about 0.5 to about 10 mg.

Aerosol formulations for treatment of the conditions referred to above, for example, migraine, in the average adult human are preferably arranged so that each metered dose or “puff” of aerosol contains about 20 μg to about 1000 μg of the compound of formula I or II. The overall daily dose with an aerosol will be within the range about 100 μg to about 10 mg. Administration may be several times daily, for example 2, 3, 4 or 8 times, giving for example, 1, 2 or 3 doses each time. Aerosol formulations containing a compound of formula I or II and a corticotropin releasing factor antagonist for treatment of the conditions referred to above in the average adult human are preferably arranged so that each metered dose or “puff” of aerosol contains about 100 μg to about 10,000 μg of the compound of formula I formula I or II and about 100 μg to about 30,000 μg of the corticotropin releasing factor antagonist. The overall daily dose with an aerosol will be within the range about 100 μg to about 20,000 mg of the compound of formula I or II and about 100 μg to about 60,000 mg of the corticotropin releasing factor antagonist. Administration may be several times daily, for example 1, 3, 4 or 8 times, giving for example, 1, 2 or 3 doses each time.

The corticotropin releasing factor antagonist and the 5-HT_{1B} receptor antagonists of formula I or II may be administered either alone or in combination with pharmaceutically acceptable carriers by either of the routes previously indicated, and such administration can be carried out in both single and multiple dosages. More particularly, this active combination can be administered in a wide variety of different dosage forms, i.e., they may be combined with various pharmaceutically-acceptable inert carriers in the form of tablets, capsules, lozenges, troches, hard candies, powders, sprays, aqueous suspension, injectable solutions, elixirs, syrups, and the like. Such carriers include solid diluents or fillers, sterile aqueous media and various non-toxic organic solvents, etc. Moreover, such oral pharmaceutical formulations can be suitably sweetened and/or flavored by means of various agents of the type commonly employed for such purposes. In general, the compounds of formula I or II are present in such dosage forms at concentration levels ranging from about 0.1% to about 95% by weight of the total composition, i.e., in amounts which are sufficient to provide the desired unit dosage, and a corticotropin releasing factor antagonist is present in such dosage forms at concentration levels ranging from about 0.1% to about 95% by weight of the total composition, i.e., in amounts which are sufficient to provide the desired unit dosage.

The corticotropin releasing factor antagonist and the 5-HT_{1B} receptor antagonists of formula I or II may be administered together or separately. When administered separately, the corticotropin releasing factor antagonists and the compounds of formula I or II may be administered in either order, provided that after administration of the first of the two active ingredients, the second active ingredient is administered within 24 hours or less, preferably 12 hours or less.

A preferred dose ratio of a corticotropin releasing factor antagonist to a compound of formula I or II in the active combination formulation for oral, parenteral or buccal administration to the average adult human for the treatment of the conditions referred to above is from about 0.001 to about 1000, preferably from about 0.001 to about 100.

It should be understood that the present invention is not limited to the embodiments described herein. Numerous modifications can be made by one skilled in the art having the benefits of the teachings given here. Such modifications should be taken as being encompassed within the scope of the present invention as set forth in the appended claims.

When referring to these preformulation compositions as homogeneous, it is meant that the active ingredients is dispersed evenly throughout the composition so that the composition may be readily subdivided into equally effective unit dosage forms such as tablets, pills and capsules. This solid preformulation composition is then subdivided into unit dosage forms of the type described above containing from about 0.1 to about 2000 mg of each of the active ingredients of the present invention. Typical unit dosage forms contain from about 1 to about 300 mg, for example about 1, 2, 5, 10, 25, 50 or 100 mg, of the active ingredient. The tablets or pills of the novel composition can be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer which serves to resist disintegration in the stomach and permits the inner component to pass intact into the duodenum or to be delayed in release. A variety of materials can be used for such enteric layers or coatings, such materials including a number of polymeric acids and mixtures of polymeric acids with such materials as shellac, cetyl alcohol and cellulose acetate.

The dosage of active ingredients in the compositions and methods of this invention may be varied; however, it is necessary that the amount of the active ingredients in such compositions be such that a suitable dosage form is obtained. The selected dosage depends upon the desired therapeutic effect, on the route of administration, the particular compounds administered, the duration of the treatment, and other factors. All dosage ranges and dosage levels mentioned herein refer to each pharmaceutically active compound present in the pharmaceutical compositions and kits of the present invention, as well as those used in the methods of the present invention. Generally, dosage levels of between about 0.0001 to about 100 mg/kg of body weight daily are administered to humans and other animals, e.g., mammals. A preferred dosage range in humans is about 0.01 to about 5.0 mg/kg of body weight daily which can be administered as a single dose or divided into multiple doses. A preferred dosage range in mammals other than humans is about 0.01 to about 10.0 mg/kg of body weight daily which can be administered as a single dose or divided into multiple doses. A more preferred dosage range in mammals other than humans is about 0.1 to about 5.0 mg/kg of body weight daily which can be administered as a single dose or divided into multiple doses.

In general, the pharmaceutical compositions, methods and kits of this invention, will be administered at dosages of a therapeutically effective amount of the first and of the second active compound in single or divided doses. The term “therapeutically effective amount” as used herein refers to a sufficient amount of the compound to treat mood disorders and psychotic disorders or conditions at a reasonable benefit/risk ratio applicable to any medical treatment.

The specific therapeutically effective dose level for any particular patient will depend upon a variety of factors including the disorder being treated and the severity of the disorder; activity of the specific compound employed; the specific composition employed; the age. However, some variation in dosage will necessarily occur depending upon the condition of the subject being treated. The person responsible for administration will, in any event, determine the appropriate dose for the individual subject.

The dosage amounts set forth in this description and in the appendant claims may be used, for example, for an average human subject having a weight of about 65 kg to about 70 kg. The skilled practitioner will readily be able to determine any variation in the dosage amount that may be required for a subject whose weight falls outside the 65 kg to 70 kg range, based upon the medical history of the subject. The pharmaceutical combinations may be administered on a regimen of up to 6 times per day, preferably 1 to 3 times per day, such as 2 times per day or once daily.

The present invention also encompasses treatment with a combination of active ingredients which may be administered separately. Accordingly, the invention also relates to combining separate pharmaceutical compositions in kit form. Thus, in one embodiment, the kit comprises two separate pharmaceutical compositions: a corticotropin releasing factor antagonist or a pharmaceutically acceptable salt of said corticotropin releasing factor antagonist; and a 5-HT_{1B} receptor antagonist of the formula I or II or a pharmaceutically acceptable salt of said 5-HT_{1B} receptor antagonist. The kit also comprises a container for containing the separate compositions such as a divided bottle or a divided foil packet, however, the separate compositions may also be contained within a single, undivided container. Typically, the kit comprises directions for the administration of the separate components. The kit form is particularly advantageous when the separate components are preferably administered in different dosage forms (e.g., oral and parenteral), are administered at different dosage intervals, or when titration of the individual components of the combination is desired by the prescribing physician. An example of such a kit is a so-called blister pack, such as a blister pack that is used in the packaging industry for the packaging of pharmaceutical unit dosage forms, including tablets, capsules, and the like. It may be desirable to provide a memory aid on the kit, e.g., in the form of numbers next to the tablets or capsules whereby the numbers correspond with the days of the regimen which the dosage form so specified should be ingested. Another example of such a memory aid is a calendar printed on the card e.g., as follows “First Week, Monday, Tuesday, . . . etc. . . . Second Week, Monday, Tuesday, . . . . ”etc. Other variations of memory aids will be readily apparent. A “daily dose” can be a single tablet or capsule or several tablets or capsules to be taken on a given day. Also, a daily dose of a corticotropin releasing factor antagonist, or a pharmaceutically acceptable salt of said corticotropin releasing factor antagonist can consist of one tablet or capsule, while a daily dose of the 5-HT_{1B} receptor antagonist of formula I or II or pharmaceutically acceptable salt of said 5-HT_{1B} receptor antagonist can consist of several tablets or capsules and vice versa. The memory aid should reflect this.

In another specific embodiment of the invention, a dispenser designed to dispense the daily doses one at a time in the order of their intended use is provided. Preferably, the dispenser is equipped with a memory-aid, so as to further facilitate compliance with the regimen. An example of such a memory-aid is a mechanical counter that indicates the number of daily doses that has been dispensed. Another example of such a memory-aid is a battery-powered micro-chip memory coupled with a liquid crystal readout, or audible reminder signal which, for example, reads out the date that the last daily dose has been taken and/or reminds one when the next dose is to be taken.

In another embodiment, the present invention comprises kits comprising a pharmaceutical composition, a package, and a package insert. The pharmaceutical composition of these kits contains either a corticotropin releasing factor antagonist or a 5-HT_{1B} receptor antagonist of formula I or II. The kits of the present invention containing a pharmaceutical composition containing a corticotropin releasing factor antagonist differ from known kits containing a pharmaceutical composition containing a corticotropin releasing factor antagonist in that on the package and/or on the package insert of the kits it is stated that the pharmaceutical composition is to be administered together with a pharmaceutical composition containing a 5-HT_{1B} receptor antagonist. The kits of the present invention containing a pharmaceutical composition containing a 5-HT_{1B} receptor antagonist of formula I or II differ from known kits containing a pharmaceutical composition containing a 5-HT_{1B} receptor antagonist in that on the package and/or on the package insert of the kits it is stated that the pharmaceutical composition is to be administered together with a pharmaceutical composition containing a corticotropin releasing factor antagonist.

The term “together with” as used in the immediately preceding paragraph is intended to encompass the simultaneous administration of the two pharmaceutical compositions (e.g., a tablet containing one pharmaceutical composition is to be administered orally while the other pharmaceutical composition is administered by way of infusion, two tablets or capsules are to be swallowed together, etc.). The term “together with” is also intended to include the administration of the two pharmaceutical compositions in a specifically timed manner, i.e., one pharmaceutical composition is to be administered a certain time period after administration of the other pharmaceutical composition. The time period in which the two pharmaceutical compositions are to be administered must be sufficiently short for the corticotropin releasing factor antagonist and the 5-HT_{1B} receptor antagonist of formula I or II to exhibit their activity contemporaneously, preferably in a synergistic manner. The exact time period depends on the specific compounds of the pharmaceutical compositions, the application route, the kind and severeness of the disease to be treated, the kind, age, and condition of the patient to be treated, etc., and can be determined by a physician using known methods in combination with the disclosure of the present invention. Generally, the two compositions are to be administered within 24 hours or less, such as 12 hours or less, preferably within 5 hours, more preferably within 2 hours, and even more preferably within one hour. Most preferably, the two compositions are to be administered at the same time or one immediately after the other.

The combinations of this invention, i.e., a corticotropin releasing factor antagonist and a 5-HT_{1B} receptor antagonist, may be tested for conditions such as, for example, migraine, depression, obsessive compulsive disorder, post-traumatic stress disorder (PTSD), and eating disorders according to the procedures described in P. P. A. Humphrey et al., Br. J. Pharmacology, 94, 1128 (1988).

The invention is further illustrated by, but by no means limited to, the following example.

### EXAMPLE 1

A pharmaceutical composition is prepared by combining 4-benzyl-2-[2-(4-methylpiperazin-1-yl)-benzylidene]-thiomorpholin-3-one, 4-(3,4-dichlorobenzyl)-2-[2-(4-methylpiperazin-1-yl)-benzylidene]-thiomorpholin-3-one, or 2-[2-(4-methylpiperazin-1-yl)-benzylidene]-4-(4-trifluoromethyl-phenyl)-thiomorpholin-3-one as the 5-HT_{1B} receptor antagonist with a CRF antagonist that is either (a) 4-(1-ethyl-propoxy)-3,6-dimethyl-2-(2,4,6-trimethylphenoxy)-pyridine, (b) (3,6-dimethyl-2-(2,4,6-trimethyl-phenoxy)-pyridin-4-yl)-(1-ethyl-propyl)-amine, (c) (3,6-dimethyl-2-(4-chloro-2,6-dimethyl-phenoxy)-pyridin-4-yl)-(1-ethyl-propyl)-amine, or (d) 5-(1-ethyl-propoxy)-7-methyl-1-(2,6-dimethyl-4-cholorophenyl)-1-4-dihydro-2H-3-oxa-1,8-diazanaphthalene in a pharmaceutically acceptable carrier. The composition contains about 0.5 mg to about 50 mg of the 5-HT_{1B} receptor antagonist and about 50 mg to about 200 mg of the CRF antagonist to deliver on a daily basis. The composition is administered to a patient for the treatment of depression on a daily, twice daily, or three times daily basis.

It should be understood that the invention is not limited to the particular embodiments described herein, but that various changes and modifications may be made without departing from the spirit and scope of this novel concept as defined by the following claims.

1. A pharmaceutical composition comprising
(i) a corticotropin releasing factor antagonist or a pharmaceutically acceptable salt thereof,
(ii) a 5-HT_{1B} receptor antagonist or a pharmaceutically acceptable salt thereof, wherein the 5-HT_{1B} receptor antagonist is selected from the group consisting of
(A) a compound of the formula I— 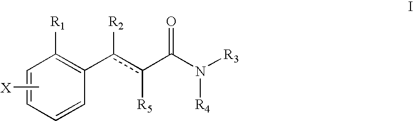
wherein, in formula I:
R¹ is a group of the formula G¹, G², G³, G⁴, G⁵, G⁶ or G⁷ depicted below, 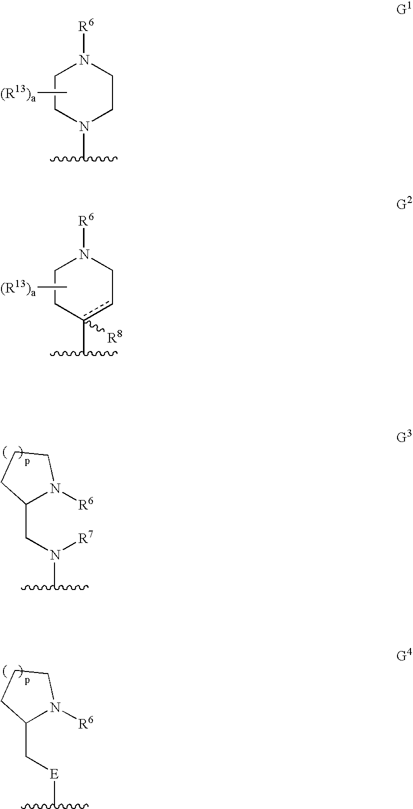
a is zero to eight;
each R¹³ is, independently, (C₁-C₄)alkyl or a (C₁-C₄)methylene bridge from one of the ring carbons of the piperazine or piperidine ring of G¹ or G², respectively, to the same or another ring carbon or a ring nitrogen of the piperazine or piperidine ring of G¹ or G², respectively, having an available bonding site, or to a ring carbon of R⁶ having an available bonding site;
E is oxygen, sulfur, SO or SO₂;
X is hydrogen, chloro, fluoro, bromo, iodo, cyano, (C₁-C₆)alkyl, hydroxy trifluoromethyl, (C₁-C₆)alkoxy, —SOₜ(C₁-C₆)alkyl wherein t is zero one or two, —CO₂R¹⁰ or —CONR¹¹R¹²,
R² is hydrogen, (C₁-C₄)alkyl, phenyl or naphthyl, wherein said phenyl or naphthyl is optionally substituted with one or more substituents independently selected from the group consisting of chloro, fluoro, bromo, iodo, (C₁-C₆)alkyl, (C₁-C₆)alkoxy, trifluoromethyl, cyano and —SOₖ(C₁-C₆)alkyl wherein k is zero, one or two;
R³ is —(CH₂)ₘB, wherein m is zero, one, two or three and B is hydrogen, phenyl, naphthyl or a 5 or 6 membered heteroaryl group containing from one to four hetero-atoms in the ring, and wherein each of the foregoing phenyl, naphthyl and heteroaryl groups is optionally substituted with one or more substituents independently selected from the group consisting of chloro, fluoro, bromo, iodo, (C₁-C₆)alkyl, (C₁-C₆)alkoxy, (C₁-C₆)alkoxy-(C₁-C₆)alkyl-, trifluoromethyl, trifluoromethoxy, cyano, hydroxy, —COOH and —SOₙ(C₁-C₆)alkyl wherein n is zero, one or two;
R⁴ is (C₁-C₆)alkyl or C₆-C₁₀ aryl;
or R³ and R⁴ may optionally be taken together with the nitrogen to which they are attached to form a five to seven membered heteroalkyl ring, wherein any two of the carbon atoms of said heteroalkyl ring is optionally replaced with a heteroatom selected from the group consisting of nitrogen, oxygen or sulfur;
R⁵ is hydrogen, (C₁-C₆)alkyl or aryl, wherein aryl is selected from the group consisting of phenyl, naphthyl, pyridyl or pyrimidyl, wherein any of said aryl is optionally independently substituted on any available bonding site by any of the radicals of X;
or R⁵ and R⁴ taken together form a divalent group —Yₙ₂—;
Y is selected from the group consisting of (a) CR⁴R⁵, wherein R⁴ and R⁵ are independently selected from hydrogen, (C₁-C₆)alkyl and trifluoromethyl; (b) a phenylene, naphthylene or a 5 or 6 membered heteroarylene ring comprising containing from one to four hetero-atoms in the heteroarylene ring, and wherein each of the foregoing phenylene, naphthylene and heteroarylene rings may optionally be substituted with one or more substituents independently selected from the group consisting of chloro, fluoro, bromo, iodo, (C₁-C₆)alkyl, (C₁-C₆)alkoxy, (C₁-C₆)alkoxy-(C₁-C₆)alkyl-, trifluoromethyl, trifluoromethoxy, cyano, hydroxy, —COOH and —SOₙ(C₁-C₆)alkyl wherein n is zero, one or two, wherein two adjacent ring atoms of ring Y are also ring atoms of ring A; and (c) an optionally substituted (C₁-C₄)heteroalkyl bridge that, together with the atoms to which it is attached, forms a five to seven membered heterocycle containing two to four heteroatoms selected from the group consisting of 1,3-oxazolidin-4-on-5-yl, 1,3-oxazolidin-2,4-dion-5-yl, 4,5-dihydro-1,2-oxazolidin-3-on-4-yl, 1,3-thiazolidin-4-on-5-yl, 1,3-thiazolidin-2,4-dion-5-yl, 1,3-pyrazolidin-4-on-5-yl, 1,3-imidazolidin-2,4-dion-5-yl, 1,2-pyrazolidin-3-on-4-yl, 1,2-thiazolidin-1,1,3-trion-4-yl, 1,2-thiazolidin-3-on-4-yl, tetrahydro-1,2-oxazin-3-on-4-yl, tetrahydro-1,3-oxazin-4-on-5-yl, tetrahydro-1,3-oxazin-2,4-dion-5-yl, morpholin-3-on-2-yl, morpholin-3,5-dion-2-yl, 2,3-dihydro-1,4-oxazin-3-on-2-yl, tetrahydro-1,3-thiazin-4-on-5-yl, tetrahydro-1,3-thiazin-2,4-d ion-5-yl, tetrahydro-1,2-thiazin-3-on-4-yl, thiomorpholin-3-on-2-yl, thiomorpholin-3,5-dion-2-yl, 2,3-dihydro-1,4-thiazin-3-on-2-yl, hexahydro-1,2-diazin-3-on-4-yl, 4,5-dihydro-2H-pyridazin-3-on-4-yl, hexahydro-1,3-diazin-4-on-5-yl, hexahydro-1,3-diazin-2,4-dion-5-yl, piperazin-2-on-3-yl, piperazin-2,6-dion-3-yl, tetrahydro-1,3,4-thiadiazin-5-on-6-yl, 5,6-dihydro-1,3,4-thiadiazin-5-on-6-yl, 1,3,4-oxadiazin-5-on-6-yl, 5,6-dihydro-1,2,4-oxadiazin-5-on-6-yl, tetrahydro-1,2,4-oxadiazin-5-on-6-yl, 1,2,4-triazin-5-on-6-yl, tetrahydro-1,2,4-oxadiazin-5-on-6-yl, 5,6-dihydro-1-2,4-oxadiazin-5-on-6-yl, 1,2,4-oxadiazin-3,5-dion-6-yl, 1,2,4-trazin-6-on-5-yl, hexahydro-1,2-oxazepin-3-on-2-yl, hexahydro-1,3-oxazepin-4-on-5-yl, hexahydro-1,4-oxazepin-3-on-2-yl, hexahydro-1,4-oxazepin-3,5-dion-2-yl, hexahydro-1,4-oxazepin-3,5-dion-6-yl, 2,3,5,6-tetrahydro-1-4-oxazepin-5,7-dion-6-yl, hexahydro-1,4-oxazepin-5-on-6-yl, hexahydro-1,3-oxazepin-2,4-dion-5-yl, hexahydro-1,2-thiazepin-3-on-4-yl, hexahydro-1,4-thiazepin-3-on-2-yl, 2,3,4,5-tetrahydro-1,4-thiazepin-3-on-2-yl, hexahydro-1,4-thiazepin-3,5-dion-2-yl, hexahydro-1,4-thiazepin-3,5-dion-6-yl, 2,3,6,7-tetrahydro-1,4-thiazepin-5-on-6-yl, 6,7-dihydro-1,4-thiazepin-5-on-6-yl, hexahydro-1,3-thiazepin-2,4-dion-5-yl, hexahydro-1,2-diazepin-3-on-4-yl, hexahydro-1,3-diazepin-2,4-dion-5-yl, hexahydro-1,4-diazepin-2-on-3-yl, hexahydro-1,4-diazepin-5-on-6-yl, hexahydro-1,4-diazepin-5,7-dion-6-yl, hexahydro-1,3,5-thiadiazepin-3-on-7-yl, 4,5,6,7-tetrahydro-1-3,5-thiadiazepin-6-on-7-yl, and 2,3,5,6-tetrahydro-1,2,4-triazepin-3,5-dion-7-yl; wherein the substituents on any of the carbon atoms capable of supporting an additional bond, of said (C₁-C₄)heteroalkyl bridge, are chloro, fluoro, (C₁-C₆)alkyl, (C₁-C₆)alkoxy, trifluoromethyl or cyano; wherein the substituents on any of the nitrogen atoms capable of supporting an additional bond, of said (C₁-C₄)heteroalkyl bridge, are (C₁-C₆)alkyl or trifluoromethyl,
n2 is one, two, three or four, with the proviso that n2 is one when Y is not CR⁴R⁵;
R⁶ is selected from the group consisting of hydrogen, (C₁-C₆)alkyl optionally substituted with (C₁-C₆)alkoxy or one to three fluorine atoms, or [(C₁-C₄)alkyl]aryl wherein the aryl moiety is phenyl, naphthyl, or heteroaryl-(CH₂)_{q}—, wherein the heteroaryl moiety is selected from the group consisting of pyridyl, pyrimidyl, benzoxazolyl, benzothiazolyl, benzisoxazolyl and benzisothiazolyl and q is zero, one, two, three or four, and wherein said aryl and heteroaryl moieties may optionally be substituted with one or more substituents independently selected from the group consisting of chloro, fluoro, bromo, iodo, (C₁-C₆)alkyl, (C₁-C₆)alkoxy, trifluoromethyl, cyano and —SO_{g}(C₁-C₆)alkyl, wherein g is zero, one or two;
R⁷ is selected from the group consisting of hydrogen, (C₁-C₆)alkyl, [(C₁-C₄)alkyl]aryl wherein the aryl moiety is phenyl, naphthyl, or heteroaryl-(CH₂)ᵣ—, wherein the heteroaryl moiety is selected from the group consisting of pyridyl, pyrimidyl, benzoxazolyl, benzothiazolyl, benzisoxazolyl and benzisothiazolyl and r is zero, one, two, three or four, and wherein said aryl and heteroaryl moieties may optionally be substituted with one or more substituents independently selected from the group consisting of chloro, fluoro, bromo, iodo, (C₁-C₆)alkyl, (C₁-C₆)alkoxy, trifluoromethyl, —C(═O)—(C₁-C₆)alkyl, cyano and —SOⱼ(C₁-C₆)alkyl, wherein j is zero, one or two;
or R⁶ and R⁷ taken together form a C₂-C₄ alkylene chain;
R⁸ is hydrogen or (C₁-C₃)alkyl;
R⁹ is hydrogen or (C₁-C₆)alkyl;
or R⁶ and R⁹, together with the nitrogen atom to which they are attached, form a 5 to 7 membered heteroalkyl ring that contains, in addition to the nitrogen atom to which R⁶ and R⁹ are attached, from zero to four heteroatoms selected from the group consisting of nitrogen, sulfur and oxygen;
and p is one, two, or three;
each of R¹⁰, R¹¹ and R¹² is selected, independently, from the groups set forth in the definition of R²; or R¹¹ and R¹², together with the nitrogen to which they are attached, form a 5 to 7 membered heteroalkyl ring that may contain, in addition to the nitrogen atom to which R¹¹ and R¹² are attached, from zero to four heteroatoms selected from the group consisting of nitrogen, sulfur and oxygen, and the broken lines indicate optional double bonds, with the proviso that when the
broken line in G² is a double bond, R⁸ is absent;
(B)
a compound of the formula II 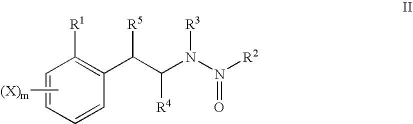
wherein in Formula II,
R¹ is a group of the formula G¹, G², G³, G⁴, G⁸ or G^{6,} wherein G¹, G², G³, G⁴, and G⁶ are each defined as for formula I, and G⁸ is depicted below 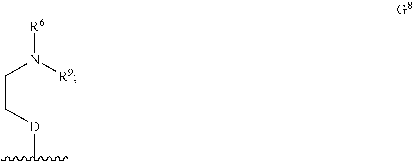
m is 0,1,2, 3 or 4;
D is oxygen, sulfur, SO, SO₂, or NR⁷;
a is zero to eight;
p is 1, 2 or 3;
E is oxygen, sulfur, SO or SO₂;
X is hydrogen, chloro, fluoro, bromo, iodo, cyano, (C₁-C₆)alkyl, hydroxy, trifluoromethyl, (C₁-C₆)alkoxy, —S(O)ₜ(C₁-C₆)alkyl wherein t is 0, 1 or 2, —CO₂R¹⁰ or —CONR¹¹R¹²;
R² is —(CH₂)ₜB, wherein t is 0, 1, 2 or 3, and B is hydrogen, phenyl, naphthyl or a 5 or 6 membered heteroaryl group containing from one to four heteroatoms in the ring, and wherein each of the foregoing phenyl, naphthyl and heteroaryl groups may optionally be substituted with one or more substituents independently selected from chloro, fluoro, bromo, iodo, (C₁-C₆)alkyl, (C₁-C₆)alkoxy, (C₁-C₆)alkoxy-(C₁-C₆)alkyl-, trifluoromethyl, trifluoromethoxy, cyano, hydroxy, —COOH and —SOₙ(C₁-C₆)alkyl wherein n is 0, 1 or 2;
R³ and R⁴ are each independently hydrogen, (C₁-C₄)alkyl or —(CH₂)_{q}-J wherein q is 0, 1, 2 or 3, and J is phenyl or naphthyl, wherein said phenyl or naphthyl may be optionally substituted with one to three substituents independently selected from the group consisting of chloro, fluoro, bromo, iodo, (C₁-C₆)alkyl, (C₁-C₆)alkoxy, trifluoromethyl, cyano and —S(O)ₖ(C₁-C₆)alkyl wherein k is 0, 1 or 2;
R⁵ is hydrogen or (C₁-C₃)alkyl;
R⁶ is selected from the group consisting of hydrogen, (C₁-C₆)alkyl optionally substituted with (C₁-C₆)alkoxy or one to three fluorine atoms, or [(C₁-C₄)alkyl]aryl wherein the aryl moiety is phenyl, naphthyl, or heteroaryl-(CH₂)_{q2}—, wherein the heteroaryl moiety is selected from the group consisting of pyridyl, pyrimidyl, benzoxazolyl, benzothiazolyl, benzisoxazolyl and benzisothiazolyl and q2 is zero, one, two, three or four, and wherein said aryl and heteroaryl moieties may optionally be substituted with one or more substituents independently selected from the group consisting of chloro, fluoro, bromo, iodo, (C₁-C₆)alkyl, (C₁-C₆)alkoxy, trifluoromethyl, cyano and —SO_{g}(C₁-C₆)alkyl, wherein g is zero, one or two;
R⁷ is selected from the group consisting of hydrogen, (C₁-C₆)alkyl, [(C₁-C₄)alkyl]aryl wherein the aryl moiety is phenyl, naphthyl, or heteroaryl-(CH₂)ᵣ—, wherein the heteroaryl moiety is selected from the group consisting of pyridyl, pyrimidyl, benzoxazolyl, benzothiazolyl, benzisoxazolyl and benzisothiazolyl and r is zero, one, two, three or four, and wherein said aryl and heteroaryl moieties may optionally be substituted with one or more substituents independently selected from the group consisting of chloro, fluoro, bromo, iodo, (C₁-C₆)alkyl, (C₁-C₆)alkoxy, trifluoromethyl, —C(═O)—(C₁-C₆)alkyl, cyano and —SOⱼ(C₁-C₆)alkyl, wherein j is zero, one or two;
or R⁶ and R⁷ taken together form a 2 to 4 carbon chain;
R⁸ is hydrogen or (C₁-C₃)alkyl;
R⁹ is hydrogen or (C₁-C₆)alkyl;
or R⁶ and R⁹, together with the nitrogen atom to which they are attached, form a 5 to 7 membered heteroalkyl ring that contains, in addition to the nitrogen atom to which R⁶ and R⁹ are attached, from zero to four heteroatoms selected from the group consisting of nitrogen, sulfur and oxygen;
each of R¹⁰, R¹¹ and R¹² is selected, independently, from the groups set forth in the definition of R³; or R¹¹ and R¹², together with the nitrogen to which they are attached, form a 5 to 7 membered heteroalkyl ring that may contain, in addition to the nitrogen atom to which R¹¹ and R¹² are attached, from zero to four heteroatoms selected from the group consisting of nitrogen, sulfur and oxygen, and
each R¹³ is, independently, (C₁-C₄)alkyl or a (C₁-C₄)methylene bridge from one of the ring carbons of the piperazine or piperidine ring of G¹ or G², respectively, to the same or another ring carbon or a ring nitrogen of the piperazine or piperidine ring of G¹ or G² respectively, having an available bonding site, or to a ring carbon of R⁶ having an available bonding site;
with the proviso that when B is hydrogen, t is not zero; and
with the proviso that when the broken line in formula G² is a double bond, R⁸ is absent;
and optionally
(iii) a pharmaceutically acceptable carrier.

2. The composition of claim 1, wherein in formula I R¹ is 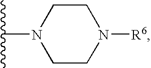
R⁶ is (C₁-C₆)alkyl, such as methyl, and R² is hydrogen.

3. The composition of claim 1, wherein in formula I R³ is hydrogen, phenyl or benzyl optionally substituted by chloro, fluoro, bromo, iodo, (C₁-C₆)alkyl or trifluoromethyl.

4. The composition of claim 1, wherein in formula I R⁴ is hydrogen or (C₁-C₆)alkyl.

5. The composition of claim 4, wherein in formula I R⁴ is methyl.

6. The composition of claim 1, wherein in formula I: R¹ is 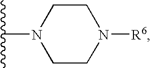
R⁶ is (C₁-C₆)alkyl and R² is hydrogen; R³ is phenyl or benzyl optionally substituted by chloro, fluoro, bromo, iodo, (C₁-C₆)alkyl or trifluoromethyl; and R⁴ is hydrogen or (C₁-C₆)alkyl.

7. The composition of claim 1, wherein in formula I R⁴ and R⁵, together with the nitrogen to which they are attached, form a 5 to 7 membered heteroalkyl ring that is selected from the group consisting of pyrrolidine, isoxazolidine, 1,3-oxazolidin-3-yl, isothiazolidine, 1,3-thiazolidin-3-yl, 1,2-pyrazolidin-2-yl, 1,3-pyrazolidin-1-yl, piperidine, thiomorpholine, 1,2-tetrahydrothiazin-2-yl, 1,3-tetrahydrothiazin-3-yl, tetrahydrothiadiazine, morpholine, 1,2-tetrahydrodiazin-2-yl, 1,3-tetrahydrodiazin-1-yl, and piperazine.

8. The composition of claim 1, wherein in formula I m is 0 or 1.

9. The composition of claim 1, wherein in formula II, R¹ is 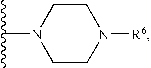
R⁶ is (C₁-C₆)alkyl and R³ is hydrogen.

10. The composition of claim 1, wherein in formula II, R¹ is 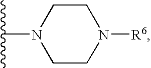
R⁶ is (C₁-C₆)alkyl and R³ is hydrogen; R² is phenyl or benzyl optionally substituted by chloro, fluoro, bromo, iodo, (C₁-C₆)alkyl or trifluoromethyl; and R⁴ is hydrogen or (C₁-C₆)alkyl.

11. The composition of claim 1, wherein the 5-HT_{1B} antagonist is selected from the group consisting of
4-benzyl-2-[2-(4-methylpiperazin-1-yl)-benzylidene]-thiomorpholin-3-one;
4-(3,4-dichlorobenzyl)-2-[2-(4-methylpiperazin-1-yl)-benzylidene]-thiomorpholin-3-one;
2-[2-(4-methylpiperazin-1-yl)-benzylidene]-4-(4-trifluoromethylphenyl)-thiomorpholin-3-one;
2-[2-(4-methylpiperazin-1-yl)-benzylidene]-thiomorpholin-3-one;
4-(3,4-dichlorophenyl)-2-[2-fluoro-6-(4-methylpiperazin-1-yl)-benzylidene]-thiomorpholin-3-one; and
4-(3,4-dichlorophenyl)-2-[2-(4-methylpiperazin-1-yl)-benzylidene]-thiomorpholin-3-one;
and a pharmaceutically acceptable salt thereof.

12. The composition of claim 1 wherein said corticotropin releasing factor antagonist is a compound of the formula 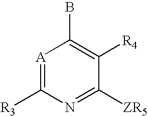
wherein
A is CR₇ or N;
B is NR₁R₂, CR₁R₂R₁₁, C(═CR₂R₁₂)R₁, NHCHR₁R₂, OCHR₁R₂, SCHR₁R₂, CHR₂OR₁₂, CHR₂SR₁₂, C(S)R₂ or C(O)R₂;
Z is NH, O, S, N(C₁-C₂ alkyl), or CR₁₃R₁₄, wherein R₁₃ and R₁₄ are each independently hydrogen, trifluoromethyl, or C₁-C₄ alkyl, or one of R₁₃ and R₁₄ may be cyano, chloro, bromo, iodo, fluoro, hydroxy, O(C₁-C₂ alkyl), amino, NH(C₁-C₂ alkyl), or CR₁₃R₁₄ may be C═O or cyclopropyl;
R₁ is C₁-C₆ alkyl which may be substituted by one or two substituents R₈ independently selected from the group consisting of hydroxy, fluoro, chloro, bromo, iodo, C₁-C₄ alkoxy, O—CO—(C₁-C₄ alkyl), O—CO—NH(C₁-C₄ alkyl), O—CO—N(C₁-C₄ alkyl)(C₁-C₂ alkyl), NH(C₁-C₄ alkyl), N(C₁-C₂ alkyl)(C₁-C₄ alkyl), S(C₁-C₄ alkyl), N(C₁-C₄alkyl)CO(C₁-C₄ alkyl), NHCO(C₁-C₄ alkyl), COO(C₁-C₄ alkyl), CONH(C₁-C₄ alkyl), CON(C₁-C₄ alkyl)(C₁-C₂ alkyl), S(C₁-C₄ alkyl), CN, NO₂, SO(C₁-C₄ alkyl), SO₂(C₁-C₄ alkyl), and said C₁-C₆ alkyl or C₁-C₄ alkyl may contain one double or triple bond;
R₂ is C₁-C₁₂ alkyl, aryl or (C₁-C₄ alkylene)aryl wherein said aryl is phenyl, naphthyl, thienyl, benzothienyl, pyridyl, quinolyl, pyrazinyl, pyrimidyl, imidazolyl, furanyl, benzofuranyl, benzothiazolyl, isothiazolyl, benzisothiazolyl, benzisoxazolyl, benzimidazolyl, indolyl, or benzoxazolyl; 3- to 8-membered cycloalkyl or (C₁-C₆ alkylene)cycloalkyl, wherein said cycloalkyl may contain one or two of O, S or N—R₉ wherein R₉ is hydrogen, or C₁-C₄ alkyl, wherein the above defined R₂ may be substituted independently by from one to three of chloro, fluoro, or C₁-C₄ alkyl, or one of bromo, iodo, C₁-C₆ alkoxy, O—CO—(C₁-C₆ alkyl), O—CO—N(C₁-C₄ alkyl)(C₁-C₂ alkyl), S(C₁-C₆ alkyl), CN, NO₂, SO(C₁-C₄ alkyl), or SO₂(C₁-C₄ alkyl), and wherein said C₁-C₁₂ alkyl or C₁-C₄ alkylene may contain one double or triple bond; or
NR₁R₂ or CR₁R₂R₁₁ may form a saturated 5- to 8-membered carbocyclic ring which may contain one or two double bonds or one or two of O or S;
R₃ is methyl, ethyl, fluoro, chloro, bromo, iodo, cyano, methoxy, OCF₃, methylthio, methylsulfonyl, CH₂OH or CH₂OCH₃;
R₄ is hydrogen, C₁-C₄ alkyl, fluoro, chloro, bromo, iodo, C₁-C₄ alkoxy, amino, nitro, NH(C₁-C₄ alkyl), N(C₁-C₄ alkyl)(C₁-C₂ alkyl), SOₙ(C₁-C₄ alkyl), wherein n is 0, 1 or 2, cyano, hydroxy, CO(C₁-C₄ alkyl), CHO, or COO(C₁-C₄ alkyl), wherein said C₁-C₄ alkyl may contain one or two double or triple bonds and may be substituted by one or two of hydroxy, amino, carboxy, NHCOCH₃, NH(C₁-C₂ alkyl), N(C₁-C₂ alkyl)₂, COO(C₁-C₄ alkyl), CO(C₁-C₄ alkyl), C₁-C₃ alkoxy, C₁-C₃ thioalkyl, fluoro, chloro, cyano or nitro;
R₅ is phenyl, naphthyl, thienyl, benzothienyl, pyridyl, quinolyl, pyrazinyl, pyrimidyl, furanyl, benzofuranyl, benzothiazolyl, or indolyl, wherein each one of the above groups R₅ is substituted independently by from one to three of fluoro, chloro, C₁-C₆ alkyl, or C₁-C₆ alkoxy, or one of hydroxy, iodo, bromo, formyl, cyano, nitro, trifluoromethyl, amino, NH(C₁-C₄ alkyl), N(C₁-C₆)(C₁-C₂ alkyl), COOH, COO(C₁-C₄ alkyl), CO(C₁-C₄ alkyl), SO₂NH(C₁-C₄ alkyl), SO₂N(C₁-C₄ alkyl)(C₁-C₂ alkyl), SO₂NH₂, NHSO₂(C₁-C₄ alkyl), S(C₁-C₆ alkyl), or SO₂(C₁-C₆ alkyl), wherein said C₁-C₄ alkyl and C₁-C₆ alkyl may be substituted by one or two of fluoro, hydroxy, amino, methylamino, dimethylamino or acetyl;
R₇ is hydrogen, C₁-C₄ alkyl, fluoro, chloro, bromo, iodo, cyano, hydroxy, O(C₁-C₄ alkyl), C(O)(C₁-C₄ alkyl), or C(O)O(C₁-C₄ alkyl), wherein the C₁-C₄ alkyl groups may be substituted with one hydroxy, chloro or bromo, or one to three fluoro;
R₁₁ is hydrogen, hydroxy, fluoro, or methoxy; and
R₁₂ is hydrogen or C₁-C₄ alkyl.

13. The composition of claim 1 wherein the corticotropin releasing factor antagonist is a compound of the formula 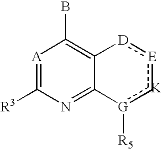
wherein the dashed lines represent optional double bonds;
A is nitrogen or CR⁷;
B is —NR¹R², —CR¹R²R¹⁰, —C(═CR²R¹¹)R¹, —NHCR¹R²R¹⁰, —OCR¹R²R¹⁰, —SCR¹R²R¹⁰, —CR²R¹⁰NHR¹, —CR²R¹⁰OR¹, —CR²R¹⁰SR¹ or —COR²;
G is nitrogen or CR⁴ and is single bonded to all atoms to which it is attached, or G is carbon and is double bonded to K;
K is nitrogen or CR⁶ when double bonded to G or E, or K is oxygen, sulfur, C═O, C═S, CR⁶R¹² or NR¹¹ when single bonded to both adjacent ring atoms, or K is a two atom spacer, wherein one of the two ring atoms of the spacer is oxygen, nitrogen, sulfur, C═O, C═S, CR⁶R¹², NR⁶ or CR⁶, and the other is CR⁶R¹² or CR⁹;
D and E are each, independently, C═O, C═S, sulfur, oxygen, CR⁴R⁶ or NR⁸ when single bonded to both adjacent ring atoms, or nitrogen or CR⁴ when it is double bonded to an adjacent ring atom;
the 6- or 7-membered ring that contains D, E, K and G may contain from one to three double bonds, from zero to two heteroatoms selected from oxygen, nitrogen and sulfur, and from zero to two C═O or C═S groups, wherein the carbon atoms of such groups are part of the ring and the oxygen and sulfur atoms are substituents on the ring;
R¹ is C₁-C₆ alkyl optionally substituted with from one or two substituents independently selected from hydroxy, fluoro, chloro, bromo, iodo, C₁-C₄ alkoxy, CF₃, —C(═O)(C₁-C₄alkyl), —C(═O)—O—(C₁-C₄)alkyl, —OC(═O)(C₁-C₄ alkyl), —OC(═O)N(C₁-C₄ alkyl)(C₁-C₂ alkyl), —NHCO(C₁-C₄ alkyl), —COOH, —COO(C₁-C₄ alkyl), —CONH(C₁-C₄ alkyl), —CON(C₁-C₄ alkyl)(C₁-C₂ alkyl), —S(C₁-C₄ alkyl), —CN, —NO₂, —SO(C₁-C₄ alkyl), —SO₂(C₁-C₄ alkyl), —SO₂NH(C₁-C₄ alkyl) and —SO₂N(C₁-C₄ alkyl)(C₁-C₂ alkyl), wherein each of the C₁-C₄ alkyl groups in the foregoing R¹ groups may optionally contain one or two double or triple bonds;
R² is C₁-C₁₂ alkyl which may optionally contain from one to three double or triple bonds, aryl or (C₁-C₄ alkylene)aryl, wherein said aryl and the aryl moiety of said (C₁-C₄ alkylene)aryl is selected from phenyl, naphthyl, thienyl, benzothienyl, pyridyl, quinolyl, pyrazinyl, pyrimidinyl, imidazolyl, furanyl, benzofuranyl, benzothiazolyl, isothiazolyl, pyrazolyl, pyrrolyl, indolyl, pyrrolopyridyl, oxazolyl and benzoxazolyl; C₃-C₈ cycloalkyl or (C₁-C₆ alkylene)(C₃-C₈ cycloalkyl), wherein one or two of the carbon atoms of said cycloalkyl and the 5 to 8 membered cycloalkyl moieties of said (C₁-C₆ alkylene)(C₃-C₈ cycloalkyl may optionally and independently be replaced by an oxygen or sulfur atom or by NZ wherein Z is hydrogen, C₁-C₄ alkyl or benzyl, and wherein each of the foregoing R² groups may optionally be substituted with from one to three substituents independently selected from chloro, fluoro, hydroxy and C₁-C₄ alkyl, or with one substituent selected from C₁-C₆ alkoxy, —OC(═O)(C₁-C₆ alkyl), —OC(═O)N(C₁-C₄ alkyl)(C₁-C₂ alkyl), —S(C₁-C₆ alkyl), amino, —NH(C₁-C₂ alkyl), —N(C₁-C₂ alkyl)(C₁-C₄ alkyl), —N(C₁-C₄ alkyl)-CO—(C₁-C₄ alkyl), —NHCO(C₁-C₄ alkyl), —COOH, —COO(C₁-C₄ alkyl), —CONH(C₁-C₄ alkyl), —CON(C₁-C₄ alkyl)(C₁-C₂ alkyl), —SH, —CN, —NO₂, —SO(C₁-C₄ alkyl), —SO₂(C₁-C₄ alkyl), —SO₂NH(C₁-C₄ alkyl) and —SO₂N(C₁-C₄ alkyl)(C₁-C₂ alkyl);
—NR¹R² or CR¹R²R¹⁰ may form a ring selected from saturated 3 to 8 membered rings, the 5 to 8 membered rings of which may optionally contain one or two double bonds, and wherein one or two of the ring carbon atoms of such 5 to 8 membered rings may optionally and independently be replaced by an oxygen or sulfur atom or by NZ² wherein Z² is hydrogen, benzyl or C₁-C₄ alkyl;
R³ is hydrogen, C₁-C₄ alkyl, —O(C₁-C₄ alkyl), chloro, fluoro, bromo, iodo, —S(C₁-C₄ alkyl) or —SO₂(C₁-C₄ alkyl);
each R⁸, R⁹ and R¹² is selected, independently, from hydrogen and C₁-C₂ alkyl;
each R⁴ and R⁶ that is attached to a carbon atom is selected, independently, from hydrogen and C₁-C₆ alkyl, fluoro, chloro, bromo, iodo, hydroxy, hydroxy(C₁-C₂ alkyl), trifluoromethyl, cyano, amino, nitro, —O(C₁-C₄ alkyl), —N(C₁-C₄ alkyl)(C₁-C₂ alkyl), —CH₂SCH₃, —S(C₁-C₄ alkyl), —CO(C₁-C₄ alkyl), —C(═O)H or —C(═O)O(C₁-C₄ alkyl), wherein each of the C₁-C₂ alkyl moieties in the foregoing R⁴ and R⁶ groups may optionally contain one double or triple bond; and R⁶, when attached to a nitrogen atom, is selected from hydrogen and C₁-C₄ alkyl;
R⁵ is substituted phenyl, naphthyl, pyridyl or pyrimidyl, wherein each of the foregoing R⁵ groups is substituted with from two to four substituents R¹³, wherein up to three of said substituents may be selected, independently, from chloro, C₁-C₆ alkyl, —O(C₁-C₆ alkyl) and —(C₁-C₆ alkylene)O(C₁-C₆alkyl), and wherein one of said substituents may be selected, independently, from bromo, iodo, formyl, cyano, trifluoromethyl, nitro, amino, —NH(C₁-C₄ alkyl), —N(C₁-C₂ alkyl)(C₁-C₆ alkyl), —C(═O)O(C₁-C₄ alkyl), —C(═O)(C₁-C₄ alkyl), —COOH, —SO₂NH(C₁-C₄ alkyl), —SO₂N(C₁-C₂ alkyl)(C₁-C₄ alkyl), —SO₂NH₂, —NHSO₂(C₁-C₄ alkyl), —(C₀-C₁alkylene)-S—(C₁-C₂ alkyl), —(C₀-C₁alkylene)-SO—(C₁-C₂alkyl), —(C₀-C₁alkylene)-SO₂-(C₁-C₂alkyl) and —(C₁-C₄ alkylene)-OH, and wherein each of the C₁-C₄ alkyl and C₁-C₆ alkyl moieties in the foregoing R⁵ groups may optionally be substituted with one or two substituents independently selected from fluoro, hydroxy, amino, methylamino, dimethylamino and acetyl;
R⁷ is hydrogen, methyl, halo, hydroxy, methoxy, —C(═O)(C₁-C₂ alkyl), —C(═O)O(C₁-C₂ alkyl), hydroxymethyl, trifluoromethyl or formyl;
R¹⁰ is hydrogen, hydroxy, methoxy or fluoro; and
R¹¹ is hydrogen or C₁-C₄ alkyl;
with the proviso that in the ring containing D, E, K and G of formula I, there can not be two double bonds adjacent to each other.

14. The composition of claim 1, wherein the corticotropin releasing factor antagonist is selected from the group consisting of:
4-(1-ethyl-propoxy)-3,6-dimethyl-2-(2,4,6-trimethylphenoxy)-pyridine;
(3,6-dimethyl-2-(2,4,6-trimethyl-phenoxy)-pyridin-4-yl)-(1-ethyl-propyl)-amine:
(3,6-dimethyl-2-(4-chloro-2,6-dimethyl-phenoxy)-pyridin-4-yl)-(1-ethyl-propyl)-amine:
5-(1-ethyl-propoxy)-7-methyl-1-(2,6-dimethyl-4-cholorophenyl)-1-4-dihydro-2H-3-oxa-1,8-diazanaphthalene;
butyl-[2,5-dimethyl-7-(2,4,6-trimethylphenyl)-6,7-dihydro-5H-pyrrolo[2,3-d]pyrimidin-4-yl]-ethyl-amino;
4-(butyl-ethylamino)-2,5-dimethyl-7-(2,4,6-trimethylphenyl)-5,7-dihydro-pyrrolo[2,3-d]pyrimidin-6-one;
4-(1-ethylpropoxy)-2,5-dimethyl-6-(2,4,6-trimethylphenoxy)-pyrimidine;
N-butyl-N-ethyl-2,5-dimethyl-NN-(2,4,6-trimethylphenyl)-pyrimidine-4,6-diamine;
[4-(1-ethyl-propoxy)-3,6-dimethyl-pyridin-2-yl]-(2,4,6-trimethylphenyl)-amine;
6-(ethyl-propyl-amino)-2,7-dimethyl-9-(2,4,6-trimethylphenyl)-7,9-dihydro-purin-8-one;
3{(4-methyl-benzyl)-[3,6-dimethyl-1-(2,4,6-trimethylphenyl)-1H-pyrazolo[3,4-d]pyrimidin-4-yl]-amino}-propan-1-ol;
diethyl-[6-methyl-3-methylsulfanyl-1-(2,4,6-trichlorophenyl)-1H-pyrazolo[3,4-d]pyrimidin-4-yl]-amine; and
2-{butyl-[6-methyl-3-methylsulfanyl-1-(2,4,6-trichlorophenyl)-1H-pyrazolo[3,4-d]pyrimidin-4-yl]-amino}-ethanol.

15. The composition of claim 14, wherein the 5-HT_{1B} antagonist is selected from the group consisting of
4-benzyl-2-[2-(4-methylpiperazin-1-yl)-benzylidene]-thiomorpholin-3-one;
4-(3,4-dichlorobenzyl)-2-[2-(4-methylpiperazin-1-yl)-benzylidene]-thiomorpholin-3-one;
2-[2-(4-methylpiperazin-1-yl)-benzylidene]-4-(4-trifluoromethylphenyl)-thiomorpholin-3-one;
2-[2-(4-methylpiperazin-1-yl)-benzylidene]-thiomorpholin-3-one;
4-(3,4-dichlorophenyl)-2-[2-fluoro-6-(4-methylpiperazin-1-yl)-benzylidene]-thiomorpholin-3-one; and
4-(3,4-dichlorophenyl)-2-[2-(4-methylpiperazin-1-yl)-benzylidene]-thiomorpholin-3-one;
and a pharmaceutically acceptable salt thereof.

16. A method for treating a disorder or condition selected from the group consisting of hypertension, depression, generalized anxiety disorder, phobias, posttraumatic stress disorder, avoidant personality disorder, sexual dysfunction, eating disorders, obesity, chemical dependencies, cluster headache, migraine, pain, Alzheimer's disease, obsessive-compulsive disorder, panic disorder, memory disorders, Parkinson's diseases, endocrine disorders, cerebellar ataxia, gastrointestinal tract disorders, negative symptoms of schizophrenia, premenstrual syndrome, Fibromyalgia Syndrome, stress incontinence, Tourette syndrome, trichotillomania, kleptomania, male impotence, cancer, chronic paroxysmal hemicrania and headache in a mammal, comprising administering to a mammal in need of such treatment components (i) and (ii) as defined in claim 1.

17. The method of claim 16 further comprising administering a 5-HT_{1A} antagonist or a pharmaceutically acceptable salt thereof, wherein the amounts of each of components (i), (ii) and the 5-HT_{1A} antagonist or a pharmaceutically acceptable salt thereof are such that the combination of components (i), (ii) and the 5-HT_{1A} antagonist or a pharmaceutically acceptable salt thereof is effective in treating the disorder or condition.

18. The method of claim 16, wherein the disorder or condition is selected from the group consisting of migraine, depression, obsessive compulsive disorder, post-traumatic stress disorder (PTSD), and eating disorders.

19. The method of claim 16, wherein component (i) is selected from the group consisting of:
4-(1-ethyl-propoxy)-3,6-dimethyl-2-(2,4,6-trimethylphenoxy)-pyridine;
(3,6-dimethyl-2-(2,4,6-trimethyl-phenoxy)-pyridin-4-yl)-(1-ethyl-propyl)-amine:
(3,6-dimethyl-2-(4-chloro-2,6-dimethyl-phenoxy)-pyridin-4-yl)-(1-ethyl-propyl)-amine:
5-(1-ethyl-propoxy)-7-methyl-1-(2,6-dimethyl-4-cholorophenyl)-1-4-dihydro-2H-3-oxa-1,8-diazanaphthalene;
butyl-[2,5-dimethyl-7-(2,4,6-trimethylphenyl)-6,7-dihydro-5H-pyrrolo[2,3-d]pyrimidin-4-yl]-ethyl-amino;
4-(butyl-ethylamino)-2,5-dimethyl-7-(2,4,6-trimethylphenyl)-5,7-dihydro-pyrrolo[2,3-d]pyrimidin-6-one;
4-(1-ethylpropoxy)-2,5-dimethyl-6-(2,4,6-trimethylphenoxy)-pyrimidine;
N-butyl-N-ethyl-2,5-dimethyl-NN-(2,4,6-trimethylphenyl)-pyrimidine-4,6-diamine;
[4-(1-ethyl-propoxy)-3,6-dimethyl-pyridin-2-yl]-(2,4,6-trimethylphenyl)-amine;
6-(ethyl-propyl-amino)-2,7-di methyl-9-(2,4,6-trimethylphenyl)-7,9-dihydro-purin-8-one;
3-{(4-methyl-benzyl)-[3,6-dimethyl-1-(2,4,6-trimethylphenyl)-1H-pyrazolo[3,4-d]pyrimidin-4-yl]-amino}-propan-1-ol;
diethyl-[6-methyl-3-methylsulfanyl-1-(2,4,6-trichlorophenyl)-1H-pyrazolo[3,4-d]pyrimidin-4-yl]-amine; and
2-{butyl-[6-methyl-3-methylsulfanyl-1-(2,4,6-trichlorophenyl)-1H-pyrazolo[3,4-d]pyrimidin-4-yl]-amino}-ethanol;
and a pharmaceutically acceptable salt thereof.

20. The method of claim 19, wherein component (ii) is selected from the group consisting of
4-benzyl-2-[2-(4-methylpiperazin-1-yl)-benzylidene]-thiomorpholin-3-one;
4-(3,4-dichlorobenzyl)-2-[2-(4-methylpiperazin-1-yl)-benzylidene]-thiomorpholin-3-one;
2-[2-(4-methylpiperazin-1-yl)-benzylidene]-4-(4-trifluoromethylphenyl)-thiomorpholin-3-one;
2-[2-(4-methylpiperazin-1-yl)-benzylidene]-thiomorpholin-3-one;
4-(3,4-dichlorophenyl)-2-[2-fluoro-6-(4-methylpiperazin-1-yl)-benzylidene]-thiomorpholin-3-one; and
4-(3,4-dichlorophenyl)-2-[2-(4-methylpiperazin-1-yl)-benzylidene]-thiomorpholin-3-one;
and a pharmaceutically acceptable salt thereof.
